(19) United States
(12) Patent Application Publication
(10) Pub. No.: US 2007/0219348 A1
(43) Pub. Date: Sep. 20, 2007

(54) Mutations in the mineralocorticoid receptor ligand binding domain polypeptide that permit structural determination of low affinity ligand complexes and screening methods employing same

(76) Inventors: Christopher James Apolito, Durham, NC (US); Randy K. Bledsoe, Durham, NC (US); Millard Hurst Lambert III, Durham, NC (US); Kevin Patrick Madauss, Durham, NC (US); Thomas B. Stanley, Durham, NC (US); Eugene Lee Stewart, Durham, NC (US); Shawn P. Williams, Durham, NC (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 11/377,502

(22) Filed: Mar. 16, 2006

(51) Int. Cl. C07K 14/715; G06F 19/00

(52) U.S. Cl. 530/350; 702/19

(57) ABSTRACT

An isolated mineralocorticoid receptor (MR) polypeptide, or functional portion thereof, having one or more mutations that alter the solubility or crystal-forming properties and confer the ability to generate soluble protein complexes with the MR and ligands that only weakly bind the native polypeptide, and a polynucleotide encoding it are disclosed. Representative mutations are C808S and S810L substitutions. Expression of the MR polypeptide in E. coli is also provided. A solved three-dimensional crystal structure of an MR ligand binding domain polypeptide is also disclosed, along with a crystalline form of the MR ligand binding domain polypeptide. Methods of modeling one or more molecular interactions of a native NR with a ligand having low affinity for the native NR utilizing a mutated MR, designing modulators of the biological activity of MR and other nuclear receptor, steroid receptor and glucocorticoid receptor polypeptides and nuclear receptor, steroid receptor and glucocorticoid receptor ligand binding domain polypeptides are also disclosed.

### TECHNICAL FIELD

The present disclosure relates generally to a modified mineralocorticoid receptor polypeptide and to a polynucleotide encoding it, to a modified mineralocorticoid receptor ligand binding domain polypeptide and to a polynucleotide encoding it. The disclosure also relates to methods by which a soluble mineralocorticoid receptor polypeptide can be generated, and by which modulators and ligands of nuclear receptors, particularly steroid receptors and the ligand binding domains thereof, can be identified and the ligand-receptor interactions characterized. The disclosure further relates to the structure of a mineralocorticoid receptor ligand binding domain, and to the structure of a mineralocorticoid receptor ligand binding domain in complex with a ligand and a co-activator.

Abbreviations

| | |
|---|---|
| ATP | adenosine triphosphate |
| ADP | adenosine diphosphate |
| AR | androgen receptor |
| CAT | chloramphenicol acyltransferase |
| CBP | CREB binding protein |
| cDNA | complementary DNA |
| DBD | DNA binding domain |
| DMSO | dimethyl sulfoxide |
| DNA | deoxyribonucleic acid |
| DTT | dithiothreitol |
| EDTA | ethylenediaminetetraacetic acid |
| ER | estrogen receptor |
| GR | glucocorticoid receptor |
| GRE | glucocorticoid responsive element |
| GST | glutathione S-transferase |
| HEPES | N-2-Hydroxyethylpiperazine-N′-2-ethanesulfonic acid |
| HSP | heat shock protein |
| kDa | kilodalton(s) |
| LBD | ligand binding domain |
| MR | mineralocorticoid receptor |
| NDP | nucleotide diphosphate |
| NID | nuclear receptor interaction domain |
| NR | nuclear receptor |
| NTP | nucleotide triphosphate |
| PAGE | polyacrylamide gel electrophoresis |
| PCR | polymerase chain reaction |
| pI | isoelectric point |
| PPAR | peroxisome proliferator-activated receptor |
| PR | progesterone receptor |
| RAR | retinoid acid receptor |
| RXR | retinoid X receptor |
| SDS | sodium dodecyl sulfate |
| SDS-PAGE | sodium dodecyl sulfate polyacrylamide gel |
| | electrophoresis |
| TIF2 | transcription intermediary factor 2 |
| TR | thyroid receptor |
| VDR | vitamin D receptor |

Amino Acid Abbreviations

| Single-Letter Code | Three-Letter Code | Name |
|---|---|---|
| A | Ala | Alanine |
| V | Val | Valine |
| L | Leu | Leucine |
| I | Ile | Isoleucine |
| P | Pro | Proline |
| F | Phe | Phenylalanine |
| W | Trp | Tryptophan |
| M | Met | Methionine |
| G | Gly | Glycine |
| S | Ser | Serine |
| T | Thr | Threonine |
| C | Cys | Cysteine |
| Y | Tyr | Tyrosine |
| N | Asn | Asparagine |
| Q | Gln | Glutamine |
| D | Asp | Aspartic Acid |
| E | Glu | Glutamic Acid |
| K | Lys | Lysine |
| R | Arg | Arginine |
| H | His | Histidine |

Functionally Equivalent Codons

| | |
|---|---|
| Amino Acid | Codons |

| | | | |
|---|---|---|---|
| Alanine | Ala | A | GCA GCC GCG GCU |
| Cysteine | Cys | C | UGC UGU |
| Aspartic Acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Methionine | Met | M | AUG |
| Asparagine | Asn | N | AAC AAU |
| Proline | Pro | P | CCA CCC CCG CCU |
| Glutamine | Gln | Q | CAA CAG |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |
| Leucine | Leu | L | UUA UUG CUA CUC |
| | | | CUG CUU |
| Arginine | Arg | R | AGA AGG CGA CGC |
| | | | CGG CGU |
| Serine | Ser | S | ACG AGU UCA UCC |
| | | | UCG UCU |

### BACKGROUND

Bacterial expression and subsequent purification of the ligand binding domains (LBDs) of the steroid receptors (SRs) for crystallography purposes has been complicated by many factors, including but not limited to low expression levels of soluble protein in the absence of ligand. In fact, bacterial expression, purification and crystallization of the progesterone (PR), androgen (AR) and glucocorticoid receptors (GR) were accomplished only when high affinity ligands were added to the growth media (Williams and Sigler 1998) (Sack et al 2001) (Bledsoe et al 2002). While this method of obtaining protein is suitable for use with high affinity ligands (e.g. having a binding affinity IC50<50 nM), lower affinity ligands (e.g. having a binding affinity IC50>50 nM) do not aid expression of the steroid receptors to the same extent, often making purification and subsequent crystallization trials impossible. In addition, while expression and purification of the receptor in the presence of a high affinity ligand followed by exchange of the high affinity ligand with a lower affinity ligand by dialysis or other approach seems plausible, in practice several complications can arise and crystallographic determination of a steroid receptor with a weakly binding ligand (e.g. having a binding affinity IC50>50 nM) has not previously been achieved.

With the determination of the steroid receptor structures described above, a general mode of steroid binding and orientation within the binding pocket of the LBD is known. However, the orientation of non-steridal ligands within the binding pocket is much more difficult to predict. What is needed are methods that will allow for rapid production and determination of crystal structures of weakly binding ligands bound in SR binding pockets. This system would be particularly useful in determining the orientation of novel, weak, non-steroidal ligands in an SR. In particular, what is needed is a surrogate receptor that has an increased affinity for ligands that bind any or all of the SRs.

### SUMMARY

It is an object of the presently disclosed subject matter to provide surrogate receptors that have increased affinity for weakly binding ligands that bind one or more NRs so as to allow modeling of molecular interactions not otherwise possible between native NRs and the weakly binding ligands.

In accordance with this object, a method of modeling one or more molecular interactions of a native NR with a ligand having low affinity for the native NR is disclosed herein. The method comprises crystallizing a surrogate ligand binding domain polypeptide in complex with a ligand having low affinity for a native NR to form a crystallized surrogate ligand binding domain polypeptide-ligand complex and analyzing the crystallized complex to determine a three-dimensional structure of the crystallized complex, whereby the three-dimensional structure of the crystallized complex models one or more molecular interactions of the native NR with the ligand. The surrogate ligand binding domain polypeptide comprises at least one mutation that improves ligand binding, crystal forming properties, or both ligand binding and crystal forming properties.

An isolated MR polypeptide comprising at least one mutation in a ligand binding domain is also disclosed. The mutation alters the solubility, ligand binding or crystallization properties of the ligand binding domain.

Further disclosed is an isolated NR polypeptide ligand binding domain, or equivalent functional portion thereof, having at least one sequence mutation at an analogous position to C808S, S810L or combinations thereof of an MR ligand binding domain based on sequence alignment to MR ligand binding domain. In some embodiments, the MR ligand binding domain comprises SEQ ID NO:4.

A method of detecting a nucleic acid molecule that encodes an MR polypeptide is also disclosed. The method comprises: (a) procuring a biological sample comprising nucleic acid material; (b) hybridizing a nucleic acid molecule encoding an MR polypeptide under stringent hybridization conditions to the biological sample of (a), thereby forming a duplex structure between the nucleic acid material within the biological sample and the nucleic acid molecule encoding an MR polypeptide; and (c) detecting the duplex structure of (b), whereby an MR encoding nucleic acid molecule is detected.

A method for identifying a substance that modulates an MR LBD function is further disclosed. The method comprises: (a) isolating an MR polypeptide comprising at least one mutation in a ligand binding domain, wherein the mutation alters the solubility, ligand binding or crystallization properties of the ligand binding domain; (b) exposing the LBD of the isolated MR polypeptide to a plurality of substances; (c) assaying binding of a substance to the LBD of the isolated MR polypeptide; and (d) selecting a substance that demonstrates specific binding to the LBD of the isolated MR polypeptide.

A method of modifying a test MR polypeptide is disclosed. The method comprises: (a) providing a test MR polypeptide sequence having a characteristic that is targeted for modification; (b) aligning the test MR polypeptide sequence with at least one reference NR polypeptide sequence for which an X-ray structure is available, wherein the at least one reference NR polypeptide sequence has a characteristic that is desired for the test MR polypeptide; (c) building a three-dimensional model for the test MR polypeptide using the three-dimensional coordinates of the X-ray structure(s) of the at least one reference NR polypeptide and its sequence alignment with the test MR polypeptide sequence; (d) examining the three-dimensional model of the test MR polypeptide for a difference in an amino acid residue as compared to the at least one reference NR polypeptide, wherein the residues are associated with the desired characteristic; and (e) mutating an amino acid residue in the test MR polypeptide sequence located at the difference identified in step (d) to a residue associated with the desired characteristic, whereby the test MR polypeptide is modified.

A method for modifying a test MR polypeptide to improve solubility in solution, ligand binding and the ability to form ordered crystals is disclosed. The method comprises providing a test MR polypeptide sequence and mutating one or more amino acid residues of the polypeptide to create a mutated polypeptide with improved solubility, ligand binding or crystal forming properties. In some embodiments, the method further comprises analyzing the mutated polypeptide for solubility, ligand binding or crystal forming properties and repeating the above steps a desired number of times until the mutated polypeptide has the desired solubility, ligand binding or crystal forming properties.

It is a further object of the presently disclosed subject matter to provide crystal structures of surrogate receptors that have increased affinity for weakly binding ligands that bind one or more NRs as well as methods for making the crystal structures. As such, a method of generating a crystallized MR ligand binding domain polypeptide is disclosed. The method comprises providing an MR ligand binding domain polypeptide comprising a mutation, wherein the mutation improves solubility, ligand binding or crystal forming properties, incubating a solution comprising the MR polypeptide with a reservoir, and crystallizing the MR polypeptide, whereby a crystallized MR ligand binding domain polypeptide is generated. Further disclosed is a crystallized MR ligand binding domain polypeptide produced by the method.

A method for determining the three-dimensional structure of a crystallized MR ligand binding domain polypeptide to a resolution of about 2.8 Å or better is disclosed. The method comprises crystallizing an MR ligand binding domain polypeptide and analyzing the MR ligand binding domain polypeptide to determine the three-dimensional structure of the crystallized MR ligand binding domain polypeptide, whereby the three-dimensional structure of the crystallized MR ligand binding domain polypeptide is determined to a resolution of about 2.8 Å or better.

A method of designing a modulator of an MR is also disclosed. The method comprises: (a) designing a potential modulator of an MR that will make interactions with amino acids in a ligand binding site of the MR based upon the atomic structure coordinates of an MR ligand binding domain polypeptide; (b) synthesizing the modulator; and (c) determining whether the potential modulator modulates the activity of the MR, whereby a modulator of an MR is designed.

A method of designing a modulator that selectively modulates the activity of an MR polypeptide is further disclosed. The method comprises: (a) obtaining a crystalline form of an MR ligand binding domain polypeptide; (b) determining the three-dimensional structure of the crystalline form of the MR ligand binding domain polypeptide; and (c) synthesizing a modulator based on the three-dimensional structure of the crystalline form of the MR ligand binding domain polypeptide, whereby a modulator that selectively modulates the activity of an MR polypeptide is designed.

A method of designing a modulator of an MR polypeptide is disclosed. The method comprises: (a) selecting a candidate MR ligand; (b) determining which amino acid or amino acids of an MR polypeptide interact with the ligand using a three-dimensional model of a crystallized protein comprising an MR LBD; (c) identifying in a biological assay for MR activity a degree to which the ligand modulates the activity of the MR polypeptide; (d) selecting a chemical modification of the ligand wherein the interaction between the amino acids of the MR polypeptide and the ligand is predicted to be modulated by the chemical modification; (e) synthesizing a chemical compound with the selected chemical modification to form a modified ligand; (f) contacting the modified ligand with the MR polypeptide; (g) identifying in a biological assay for MR activity a degree to which the modified ligand modulates the biological activity of the MR polypeptide; and (h) comparing the biological activity of the MR polypeptide in the presence of the modified ligand with the biological activity of the MR polypeptide in the presence of the unmodified ligand, whereby a modulator of an MR polypeptide is designed.

A method of screening a plurality of compounds for a modulator of an MR ligand binding domain polypeptide is disclosed. The method comprises: (a) providing a library of test samples; (b) contacting an MR ligand binding domain polypeptide with each test sample; (c) detecting an interaction between a test sample and the MR ligand binding domain polypeptide; (d) identifying a test sample that interacts with the MR ligand binding domain polypeptide; and (e) isolating the test sample that interacts with the MR ligand binding domain polypeptide, whereby a plurality of compounds is screened for a modulator of an MR ligand binding domain polypeptide.

An assay method for identifying a compound that inhibits binding of a ligand to an MR polypeptide is also disclosed. The assay method comprises: (a) designing a test inhibitor compound based on the three dimensional atomic coordinates of MR; (b) incubating an MR polypeptide with a ligand in the presence of a test inhibitor compound; (c) determining an amount of ligand that is bound to the MR polypeptide, wherein decreased binding of ligand to the MR polypeptide in the presence of the test inhibitor compound relative to binding of ligand in the absence of the test inhibitor compound is indicative of inhibition; and (d) identifying the test compound as an inhibitor of ligand binding if decreased ligand binding is observed, whereby a compound that inhibits binding of a ligand to an MR polypeptide is identified.

Some of the objects of the presently disclosed subject matter having been stated hereinabove, and which are addressed in whole or in part by the present disclosure, other objects will become evident as the description proceeds, when taken in connection with the accompanying Laboratory Examples as best described hereinbelow.

### BRIEF DESCRIPTION OF THE DRAWINGS

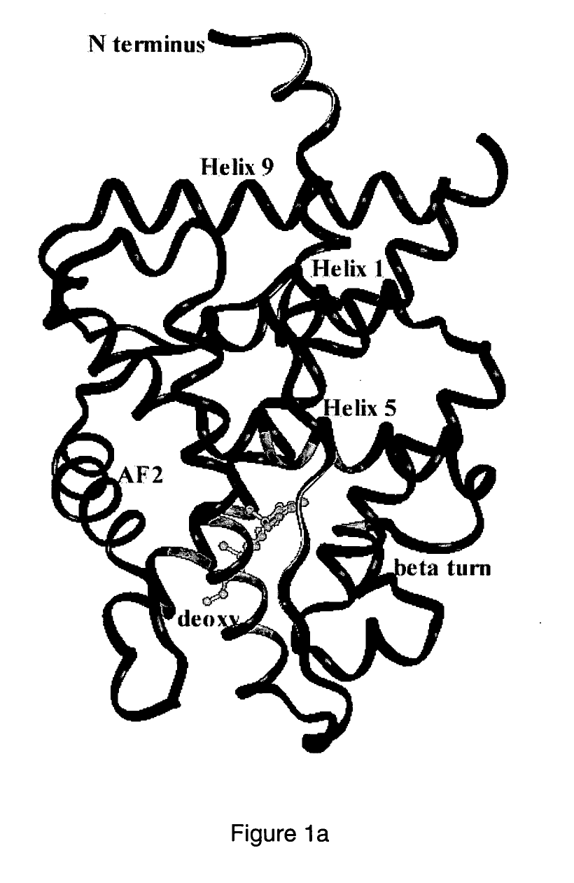

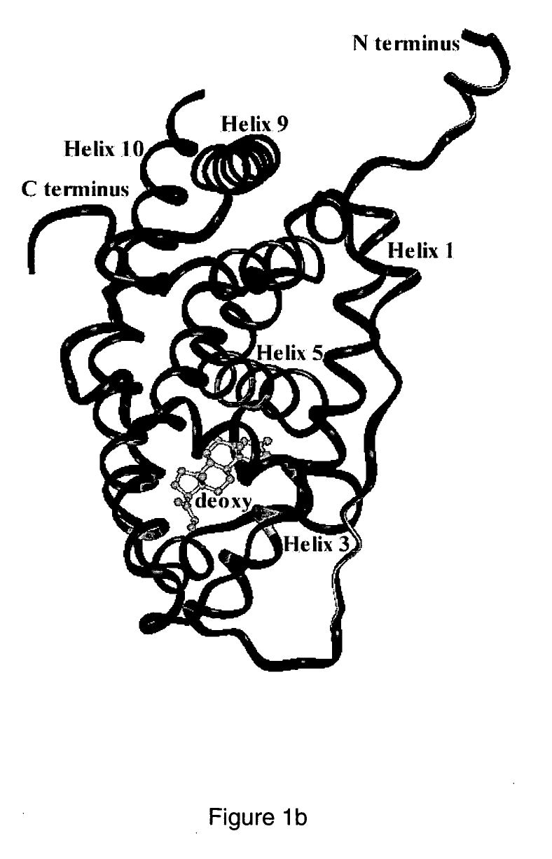

Sheet 1/18, hereinafter FIG. 1A, depicts the structure of the wild type or native mineralocorticoid receptor as a ribbon diagram. The diagram illustrates that the fold of MR is the same as other steroid receptors, with deoxycorticosterone bound in an internal pocket. MR is shown in black, and residues contacting the ligand are colored gray. Deoxycorticosterone is shown as a gray stick-and-ball figure.

Sheet 2/18, hereinafter FIG. 1B, depicts the same structure as FIG. 1A, rotated 90°.

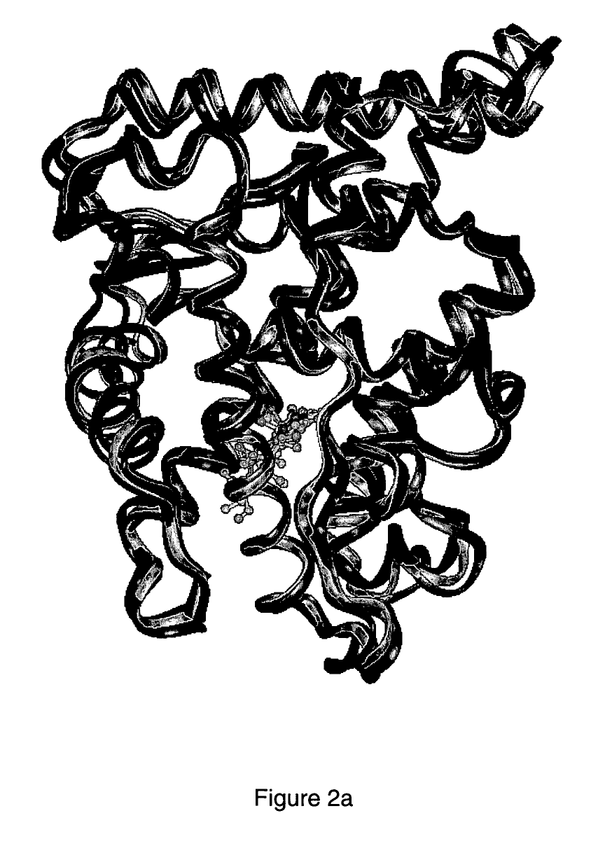

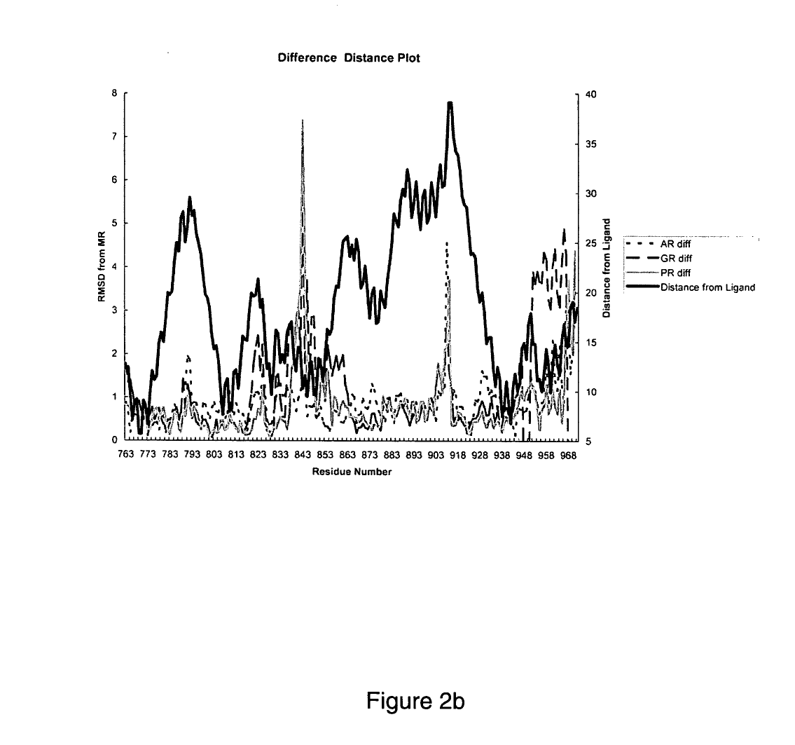

Sheet 3/18, hereinafter FIG. 2A, depicts overlapping ribbon diagrams showing the alignment of MR (black) with AR (dark gray), GR (gray), and PR (light gray).

Sheet 4/18, hereinafter FIG. 2B, depicts a difference distance plot showing the rms. deviation between the Cα of MR with those of AR, GR, and PR (left Y axis) from the beginning of helix 3 to the end of helix 12. The overlaid graph (right Y axis) shows the distance between the MR Cα and the center of mass of deoxycorticosterone.

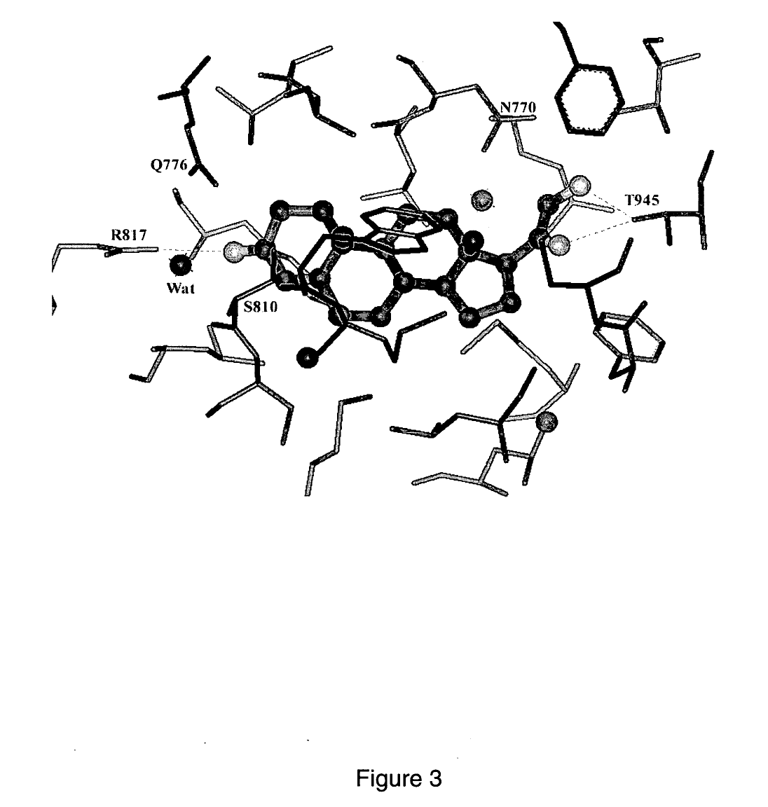

Sheet 5/18, hereinafter FIG. 3, depicts the MR binding pocket with deoxycorticosterone bound therein. Amino acid residues binding to deoxycorticosterone are named. MR is shown as a gray stick figure with deoxycorticosterone as a ball and stick model. Deoxycorticosterone carbon and oxygen atoms are colored black and light gray, respectively. Water molecules are shown as dark gray spheres. Hydrogen bonds are shown as dashed lines.

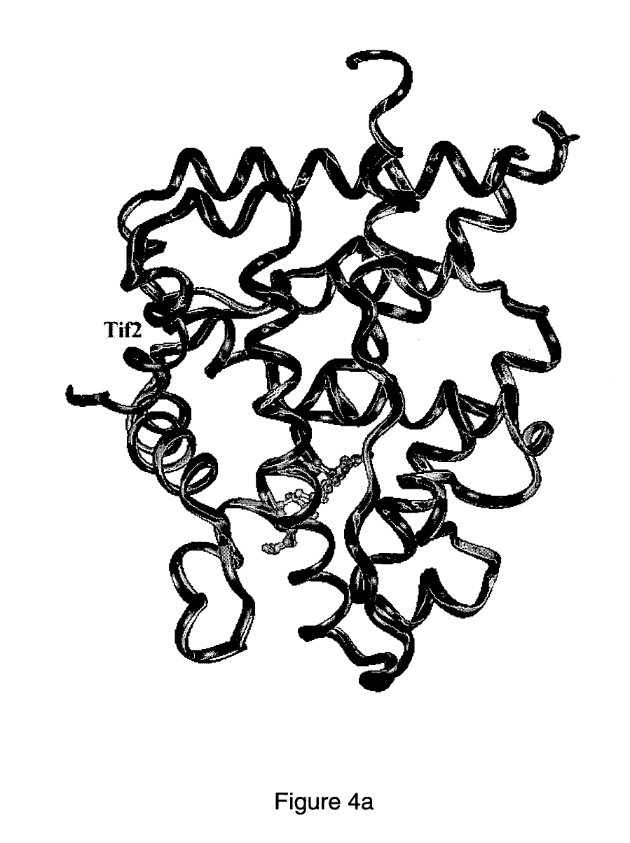

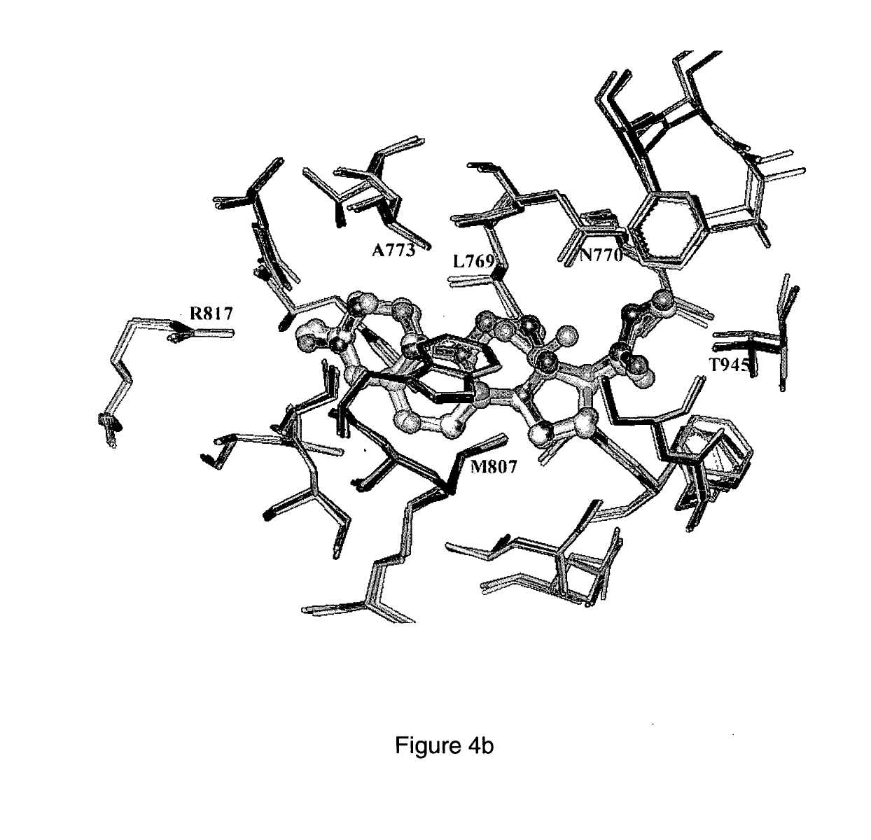

Sheet 6/18, hereinafter FIG. 4A, depicts ribbon diagrams showing a comparison of native MR to MR C808S with bound agonist. The overall structure of MR changes insignificantly between the native and the C808S mutant protein. Ribbon diagrams show the conformation of native MR/deoxycorticosterone (black), MR C808S/deoxycorticosterone without (dark gray) and with (gray) TIF2 peptide, and MR C808S/aldosterone (light gray).

Sheet 7/18, hereinafter FIG. 4B, depicts the binding pocket of native MR in comparison with MR C808S complexed with deoxycorticosterone with or without TIF2. The conformations of binding pocket residues of native MR (black) are the same as MR with cysteine 808 mutated to serine, whether complexed with deoxycorticosterone without (dark gray) or with (gray) TIF2 peptide, or aldosterone (light gray). MR proteins are shown as stick figures, and the bound ligand is shown as a ball and stick figure.

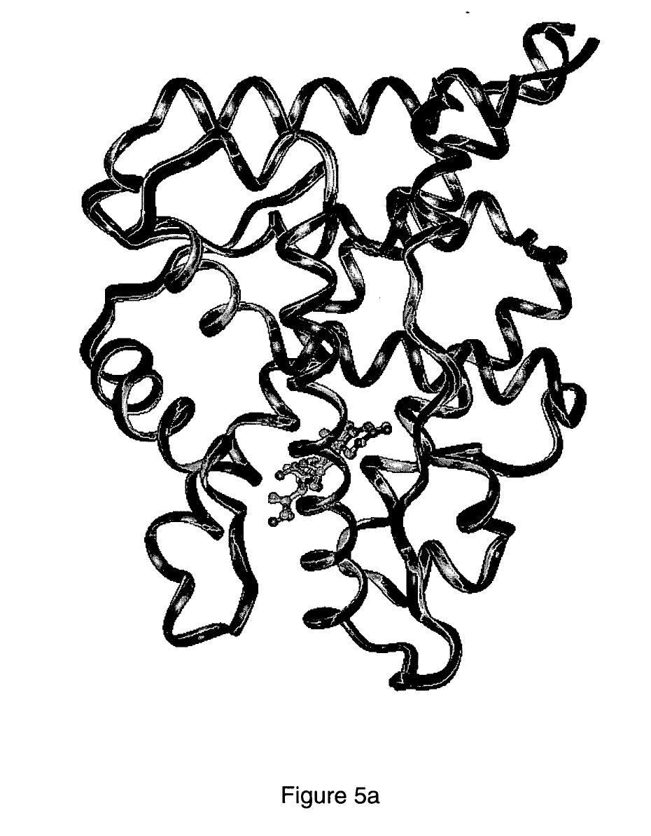

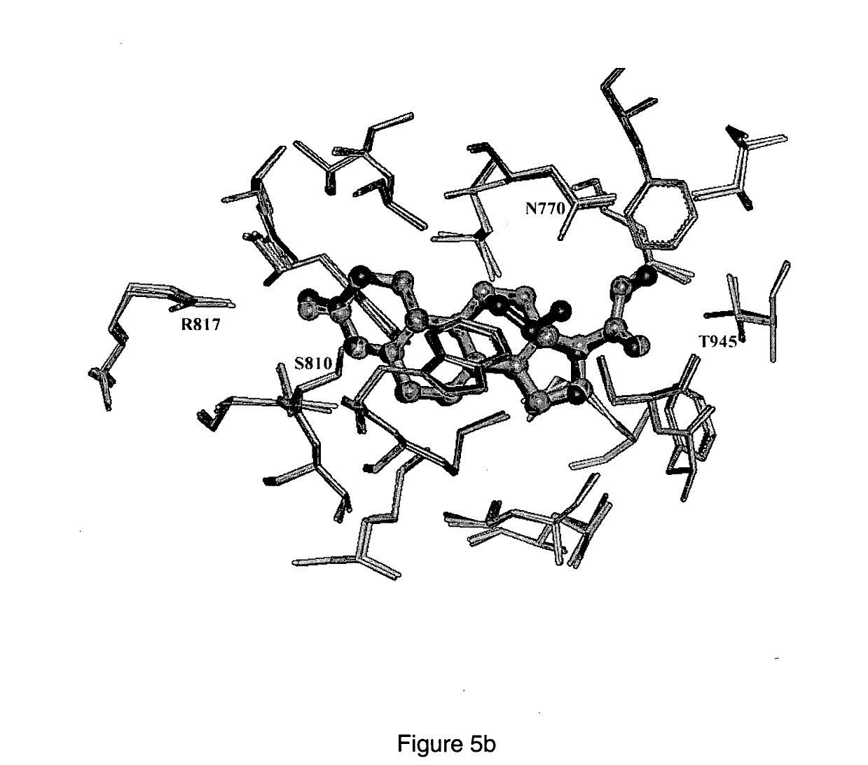

Sheet 8/18, hereinafter FIG. 5A, depicts the interaction of MR with the partial agonist progesterone. Ribbon diagrams comparison of MR (C808S) bound to aldosterone (black ribbon and black ball-and-stick compound) and the two progesterone molecules (gray ribbon and ball-and-stick compound).

Sheet 9/18, hereinafter FIG. 5B, depicts the interaction of MR with the partial agonist progesterone at the binding pocket. Comparison of the binding pockets shows that the progesterone conformation (light gray stick protein and ball-and-stick compound) is very similar to that of aldosterone (black stick protein and ball-and-stick compound).

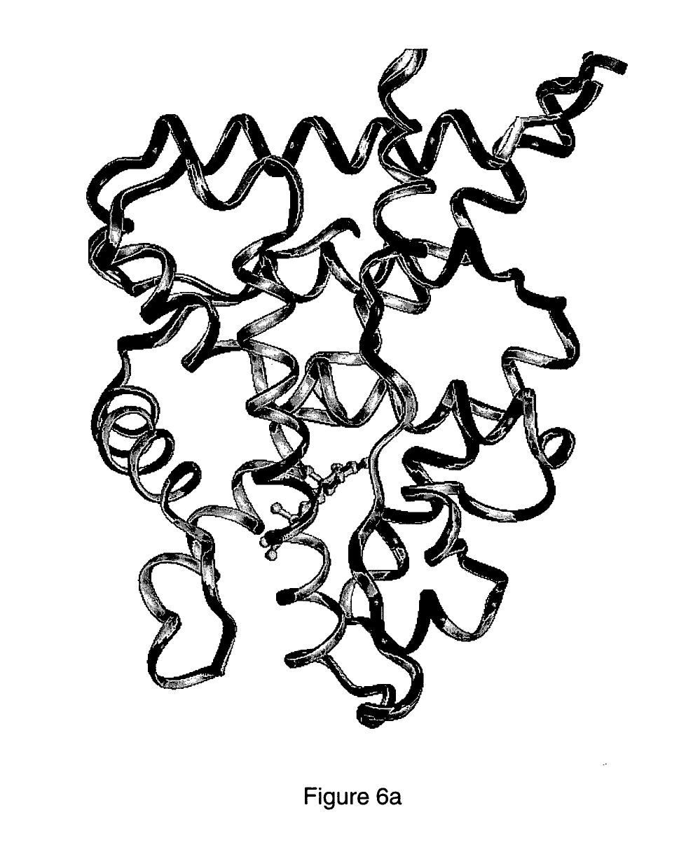

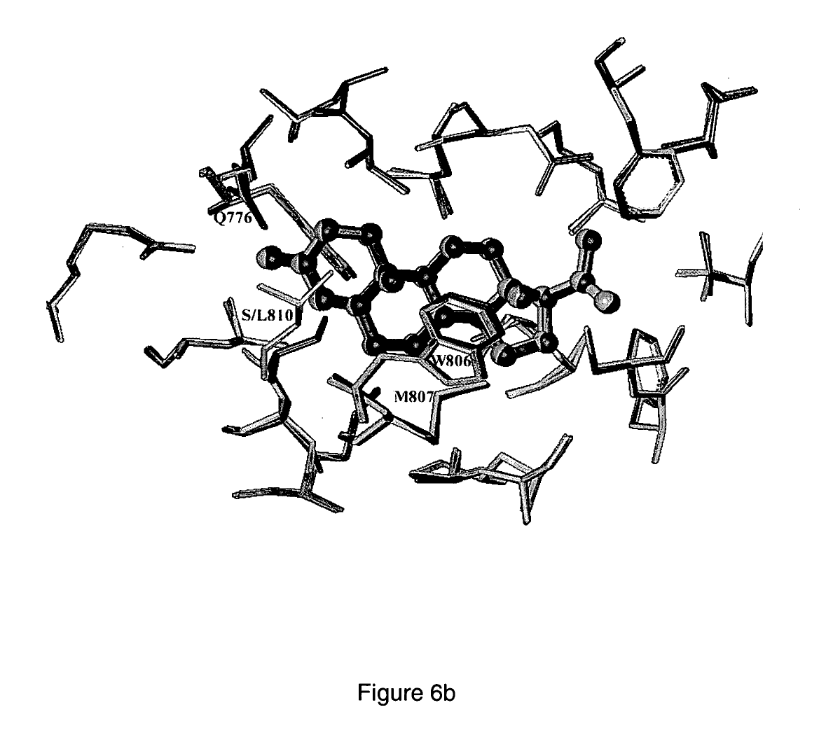

Sheet 10/18, hereinafter FIG. 6A, depicts a comparison of MR single and double mutants complexed with progesterone. A ribbon diagram illustrates that the single (black) and double (light gray) mutants have superimposable main chain conformations.

Sheet 11/18, hereinafter FIG. 6B, depicts a comparison of the binding pockets showing that the conformation of residues surrounding progesterone (ball and stick) is nearly identical for the single (white) and double (gray) MR mutants.

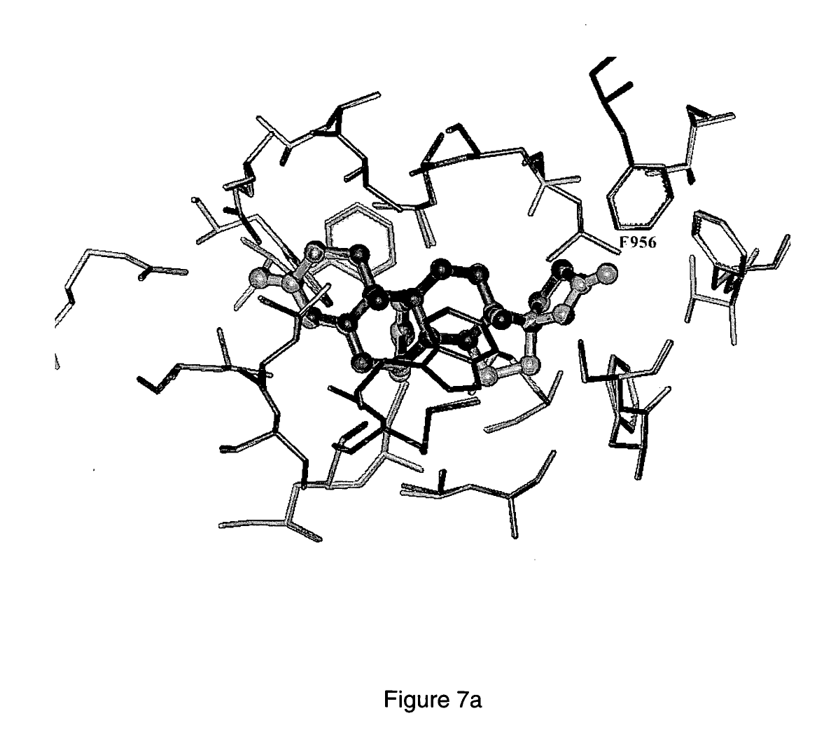

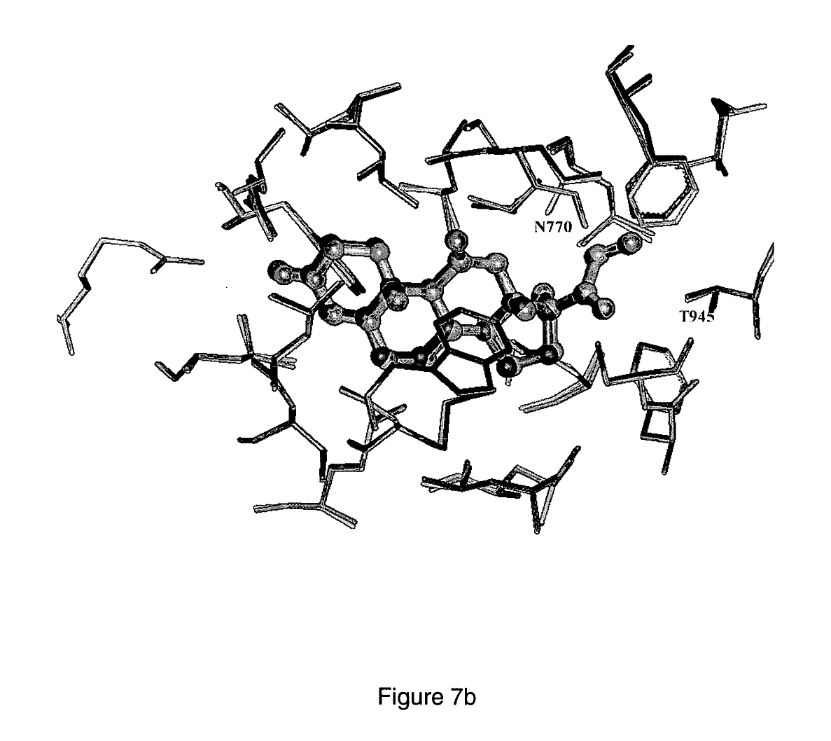

Sheet 12/18, hereinafter FIG. 7A, depicts the MR double mutant complexed with spironolactone. The conformation of residues surrounding spironolactone in the two MR molecules (black stick protein and ball and stick ligand and light gray stick protein and ball and stick ligand) is nearly identical.

Sheet 13/18, hereinafter FIG. 7B, depicts the MR double mutant complexed with cortisone. The conformation of residues surrounding cortisone in the two MR molecules (black stick protein and ball and stick ligand and light gray stick protein and ball and stick ligand) is nearly identical.

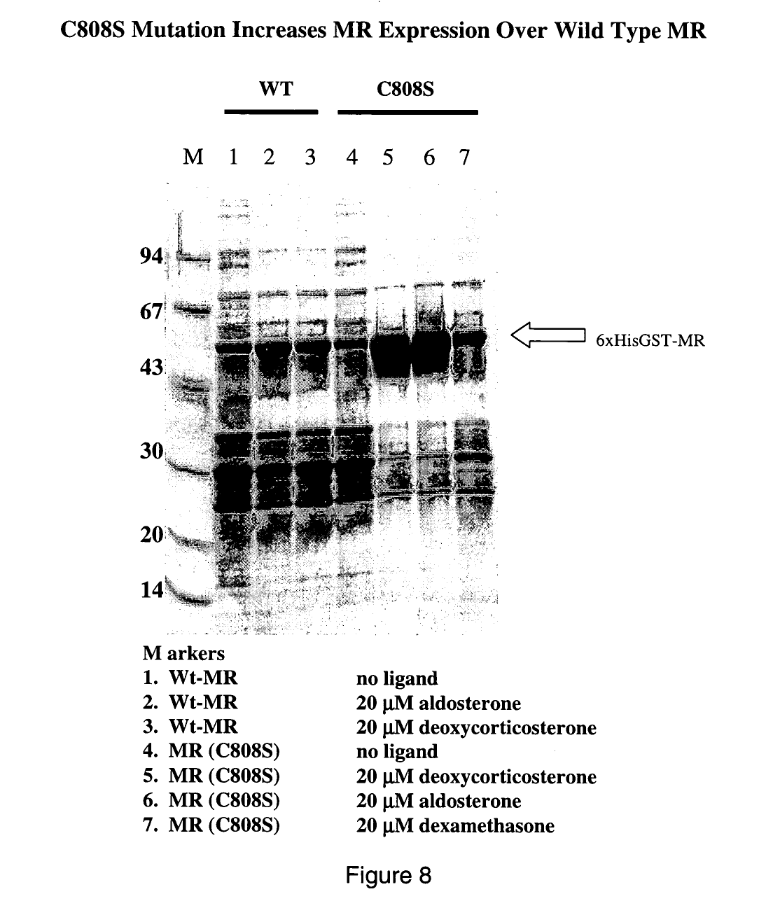

Sheet 14/18, hereinafter FIG. 8, is a protein gel showing E. coliexpression of mutant 6×HisGST-MR(712-984) C808S versus 6×HisGST-wild type MR.

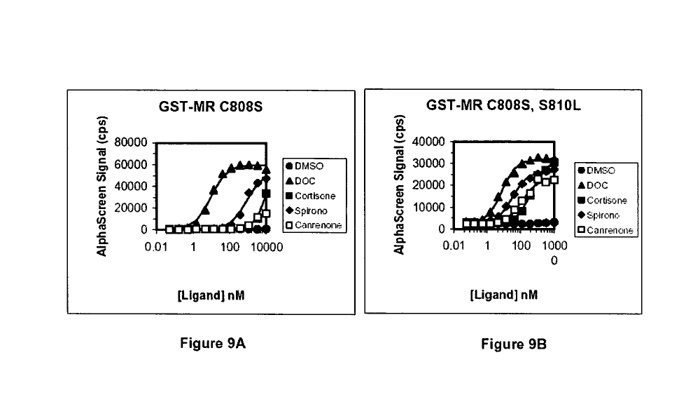

Sheet 15/18, hereinafter FIG. 9A, is a graph showing the effects of different ligands on the binding and activation of 3 nM TIF2 LXXLL-containing coactivator peptide (SEQ ID NO:11) to 0.3 nM 6× HisGST-MR C808S (SEQ ID NO:6) LBD.

Sheet 16/18, hereinafter FIG. 9B, is a graph showing the effects of different ligands on the binding and activation of 3 nM TIF2 LXXLL-containing coactivator peptide (SEQ ID NO:11) to 0.5 nM 6× His GST-MR C808S,S810L LBD (SEQ ID NO:10).

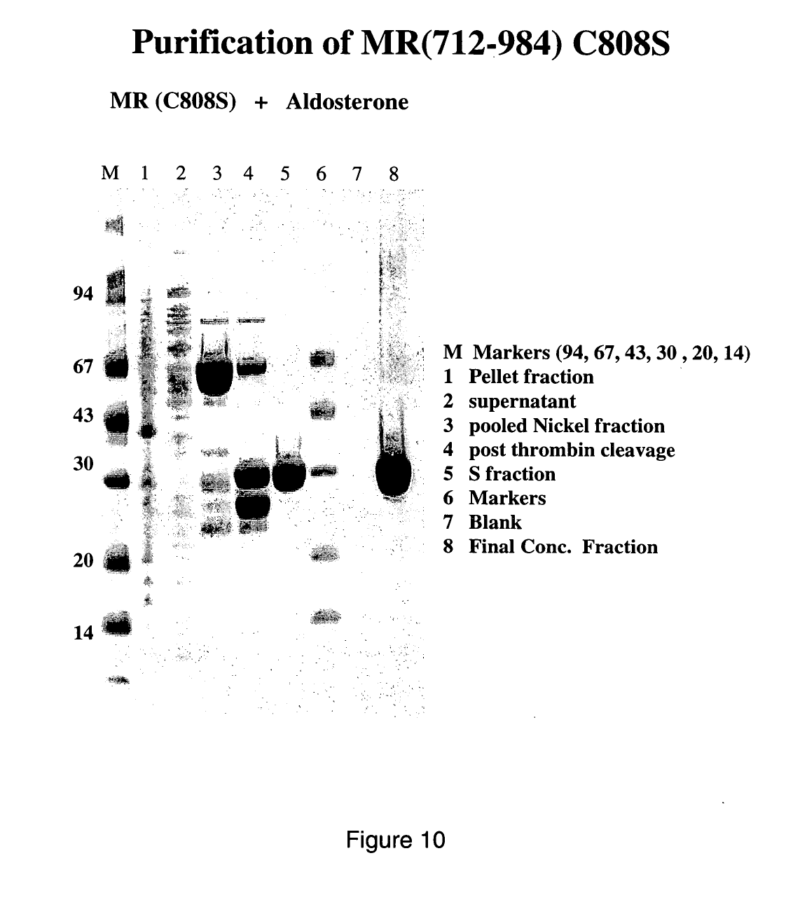

Sheet 17/18, hereinafter FIG. 10, is a protein gel showing purification of the E. coli expressed MR(712-984) C808S bound with aldosterone by SDS PAGE.

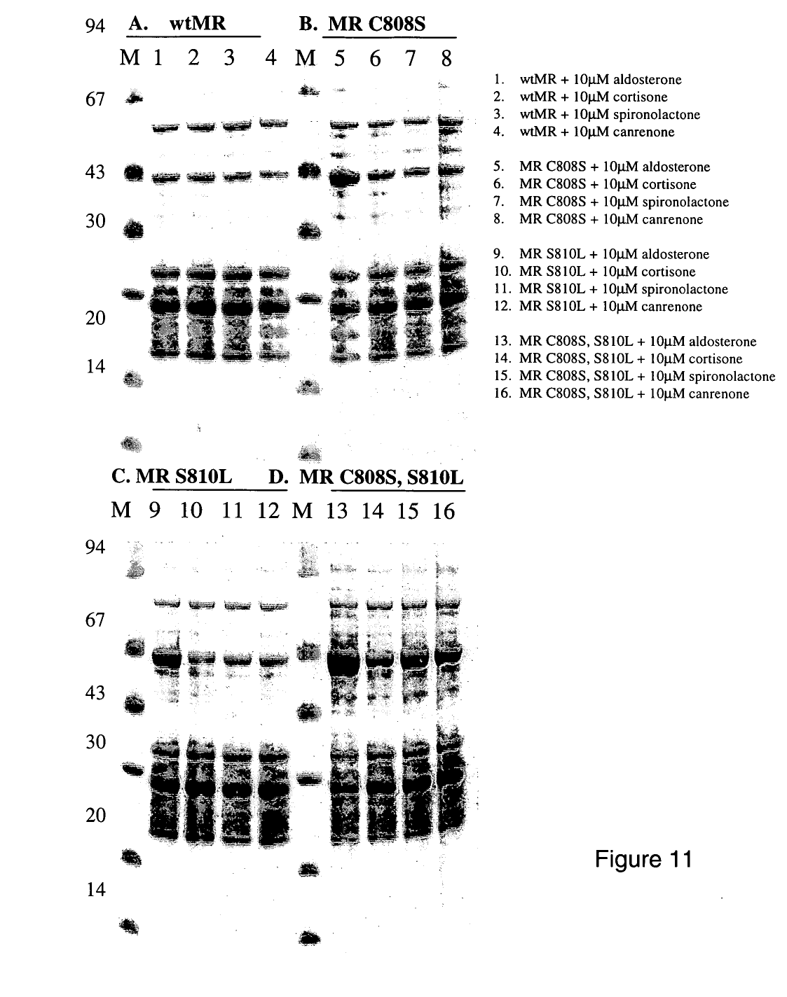

Sheet 18/18, hereinafter FIGS. 11A-11D, are a set of protein gels showing E. coli expression of wild type (wt) MR (11A) versus mutant 6×HisGST-MR(712-984) C808S (11B) versus 6×HisGST-MR(712-984) S810L (11C) and the combination mutant 6×HisGST-MR(712-984) C808S, S810L (11D) in the presence of the listed ligands. Lanes: (1) wtMR+1 0 mM aldosterone; (2) wtMR +10 mM cortisone; (3) wtMR+10 mM spironolactone; (4) wtMR+10 mM canrenone; (5) MR C808S+10 mM aldosterone; (6) MR C808S+10 mM cortisone; (7) MR C808S+10 mM spironolactone; (8) MR C808S+10 mM canrenone; (9) MR S810L+10 mM aldosterone; (10) MR S810L+10 mM cortisone; (11) MR S810L+10 mM spironolactone; (12) MR S810L+10 mM canrenone; (13) MR C808S, S810L+10 mM aldosterone; (14) MR C808S, S810L+10 mM cortisone; (15) MR C808S, S810L+l0mM spironolactone; (16) MR C808S, S810L+10 mM canrenone.

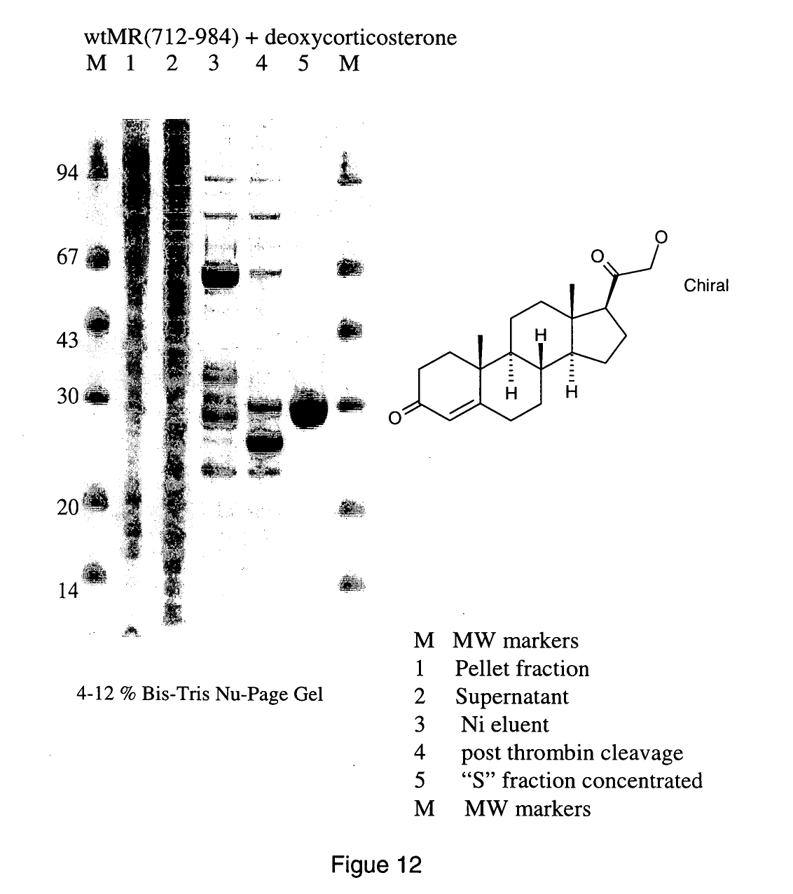

Sheet 19/19, hereinafter FIG. 12, is a protein gel showing purification of the E. coli expressed MR(712-984) C808S bound with deoxycorticosterone by SDS PAGE.

### BRIEF DESCRIPTION OF SEQUENCES IN THE SEQUENCE LISTING

SEQ ID NOs:1 and 2 are, respectively, a DNA sequence encoding a wild type full-length human mineralocorticoid receptor (GENBANK® Accession No. M16801) and the amino acid sequence of a human mineralocorticoid receptor (GENBANK® Accession No. AAA59571.1) encoded by the DNA sequence.

SEQ ID NOs:3 and 4 are, respectively, a DNA sequence encoding a wild type ligand binding domain of a human mineralocorticoid receptor and the amino acid sequence of a human mineralocorticoid receptor encoded by the DNA sequence.

SEQ ID NOs:5 and 6 are, respectively, a DNA sequence encoding a ligand binding domain (residues 712-984) of a human mineralocorticoid receptor containing a cysteine to serine mutation at residue 808 and the amino acid sequence of a human mineralocorticoid receptor encoded by the DNA sequence.

SEQ ID NOs:7 and 8 are, respectively, a DNA sequence encoding a ligand binding domain (residues 712-984) of a human mineralocorticoid receptor containing a serine to leucine mutation at residue 810 and the amino acid sequence of a human mineralocorticoid receptor encoded by the DNA sequence.

SEQ ID NOs:9 and 10 are, respectively, a DNA sequence encoding a ligand binding domain (residues 712-984) of a human mineralocorticoid receptor containing a cysteine to serine mutation at residue 808 and a serine to leucine mutation at residue 810 and the amino acid sequence of a human mineralocorticoid receptor encoded by the DNA sequence.

SEQ ID NO:11 is an amino acid sequence of amino acid residues 732-756 of the human TIF2 protein.

SEQ ID NO:12 is an LXXLL motif of the human TIF2 protein.

SEQ ID NOs:13 and 14 are, respectively, forward and reverse oligonucleotide primers used to engineer mutant MR LBD (C808S).

SEQ ID NOs:15 and 16 are, respectively, forward and reverse oligonucleotide primers used to engineer mutant MR LBD (C808S, S810L).

SEQ ID NOs:17 and 18 are, respectively, the final resultant sequences of the purified mutant proteins MR LBD (C808S) and MR LBD (C808S, S810L) after digestion at the thrombin cleavage site.

### DETAILED DESCRIPTION

The presently disclosed subject matter provides for the generation of NR polypeptides and NR mutants (in some embodiments MR LBD mutants), that confer the ability to generate soluble protein complexes with ligands that only weakly bind the native polypeptide. The disclosed subject matter also provides the ability to perform crystallization trials on these weakly binding ligands and the ability to solve the crystal structures of those that crystallize. Indeed, MR LBDs having one or more point mutations were crystallized and solved in one aspect of the presently disclosed subject matter. Based on the fact that MR can bind androgens, progestins and glucocorticoids, the MR mutant polypeptides disclosed herein can serve as surrogates for the native receptors of these ligands and allow for the determination of crystal structures of the mutant MR bound to weak ligands that bind the other receptors, and, which cannot be obtained with the AR, PR and GR constructs currently in existence.

Thus, an aspect of the presently disclosed subject matter involves the use of both targeted and random mutagenesis of the MR gene for the production of a recombinant protein with improved solution characteristics for the purpose of crystallization, characterization of biologically relevant protein-protein interactions, and compound screening assays. The presently disclosed subject matter, relating to MR LBD C808S+S810L and other LBD mutations, shows that MR can be overexpressed using an E. coli expression system and that active MR protein can be purified, assayed, and crystallized.

Polypeptides, including the MR LBD, have a three-dimensional structure determined by the primary amino acid sequence and the environment surrounding the polypeptide. This three-dimensional structure establishes the polypeptide's activity, stability, binding affinity, binding specificity, and other biochemical attributes. Thus, knowledge of a protein's three-dimensional structure can provide much guidance in designing agents that mimic, inhibit, or improve its biological activity.

The three-dimensional structure of a polypeptide can be determined in a number of ways. Many of the most precise methods employ X-ray crystallography (See, e.g., Van Holde, (1971) Physical Biochemistry, Prentice-Hall, New Jersey, pp. 221-39). This technique relies on the ability of crystalline lattices to diffract X-rays or other forms of radiation. Diffraction experiments suitable for determining the three-dimensional structure of macromolecules typically require high-quality crystals. Unfortunately, such crystals have been unavailable for the ligand binding domain of a human MR, as well as many other proteins of interest. Thus, high-quality diffracting crystals of the ligand binding domain of a human MR in complex with a ligand and a peptide would greatly assist in the elucidation of its three-dimensional structure.

The solved crystal structure of the LBD of an MR polypeptide would be useful in the design of modulators of activity mediated by the mineralocorticoid receptor. Additionally, evaluation of the available sequence data shows that MR is particularly similar to progesterone receptor (PR), glucocorticoid receptor a (GRα) and androgen receptor (AR). The MR LBD has approximately 57%, 56% and 51% sequence identity to the PR, GRα and AR LBDs, respectively. Based on the fact that MR can bind androgens, progestins and glucocorticoids, the MR mutant polypeptides disclosed herein can serve as a surrogate for these receptors and allow for the determination of crystal structures that cannot be obtained with the AR, PR and GR constructs currently in existence.

The solved MR crystal structure would also provide structural details and insights necessary to design a modulator of MR that maximizes preferred requirements for any modulator, i.e. potency and specificity. By exploiting the structural details obtained from an MR-ligand-co-activator crystal structure, it would be possible to design an MR modulator that, despite MR's similarity with other steroid receptors and nuclear receptors, exploits the unique structural features of the ligand binding domain of human MR.

It would also be desirable to analyze and understand the interaction of MR and other NRs with weakly binding ligands (e.g. having a binding affinity IC50>50 nM). Such information would be applicable to determining how weakly binding ligands, such as cross-reactive drugs, affect NR function. Unfortunately, until the present disclosure it has proven very difficult to obtain adequate expression levels of NR LBDs in combination with weakly binding ligands for crystallography purposes. As such, crystallographic determination of a nuclear receptor, including particularly a steroid receptor, with a weakly binding ligand has heretofore not been achieved. The present disclosure teaches methods for obtaining crystal structures of NR LBDs in combination with weakly binding ligands.

Until disclosure of the present subject matter, the ability to obtain crystalline forms of the ligand binding domain of MR had not been realized. And until disclosure of the subject matter presented herein, a detailed three-dimensional crystal structure of a MR LBD polypeptide had not been solved. Further, until disclosure of the present subject matter, a detailed three-dimensional crystal structure of a MR LBD polypeptide bound with a weakly binding ligand (e.g. having a binding affinity IC50>50 nM) had not been achieved.

In addition to providing structural information, crystalline polypeptides provide other advantages. For example, the crystallization process itself further purifies the polypeptide, and satisfies one of the classical criteria for homogeneity. In fact, crystallization frequently provides unparalleled purification quality, removing impurities that are not removed by other purification methods such as HPLC, dialysis, conventional column chromatography, and other methods. Moreover, crystalline polypeptides are sometimes stable at ambient temperatures and free of protease contamination and other degradation associated with solution storage. Crystalline polypeptides can also be useful as pharmaceutical preparations. Finally, crystallization techniques in general are largely free of problems such as denaturation associated with other stabilization methods (e.g., lyophilization). Once crystallization has been accomplished, crystallographic data provides useful structural information that can assist the design of compounds that can serve as modulators (e.g. agonists or antagonists), as described herein below. In addition, the crystal structure provides information useful to map a receptor binding domain, which can then be mimicked by a chemical entity that can serve as an antagonist or agonist.

I. Definitions

Following long-standing patent law convention, the terms “a” and “an” mean “one or more” when used in this application, including the claims.

As used herein, the term “agonist” means an agent that supplements or potentiates the bioactivity of a functional NR gene or protein, or of a polypeptide encoded by a gene that is up- or down-regulated by an NR polypeptide and/or a polypeptide encoded by a gene that contains an NR binding site or response element in its promoter region. By way of specific example, an “agonist” is a compound that interacts with the steroid hormone receptor to promote a transcriptional response. An agonist can induce changes in a receptor that places the receptor in an active conformation that allows them to influence transcription, either positively or negatively. There can be several different ligand-induced changes in the receptor's conformation. The term “agonist” specifically encompasses partial agonists.

As used herein, the terms “α-helix”, “alpha-helix” and “alpha helix” are used interchangeably and mean the conformation of a polypeptide chain wherein the polypeptide backbone is wound around the long axis of the molecule in a left-handed or right-handed direction, and the R groups of the amino acids protrude outward from the helical backbone, wherein the repeating unit of the structure is a single turn of the helix, which extends about 0.56 nm along the long axis.

As used herein, the term “antagonist” means an agent that decreases or inhibits the bioactivity of a functional NR gene or protein, or that supplements or potentiates the bioactivity of a naturally occurring or engineered non-functional NR gene or protein. Alternatively, an antagonist can decrease or inhibit the bioactivity of a functional gene or polypeptide encoded by a gene that is up- or down-regulated by an NR polypeptide and/or contains an NR binding site or response element in its promoter region. An antagonist can also supplement or potentiate the bioactivity of a naturally occurring or engineered non-functional gene or polypeptide encoded by a gene that is up- or down-regulated by an NR polypeptide, and/or contains an NR binding site or response element in its promoter region. By way of specific example, an “antagonist” is a compound that interacts with the steroid hormone receptor to inhibit a transcriptional response. An antagonist can bind to a receptor but fail to induce conformational changes that alter the receptor's transcriptional regulatory properties or physiologically relevant conformations. Binding of an antagonist can also block the binding and therefore the actions of an agonist. The term “antagonist” specifically encompasses partial antagonists.

As used herein, the terms “P-sheet”, “beta-sheet” and “beta sheet” are used interchangeably and mean the conformation of a polypeptide chain stretched into an extended zigzag conformation. Portions of polypeptide chains that run “parallel” all run in the same direction. Polypeptide chains that are “antiparallel” run in the opposite direction from the parallel chains.

As used herein, the terms “binding pocket of the NR ligand binding domain”, “NR ligand binding pocket” and “NR binding pocket” are used interchangeably, and refer to the cavity within the NR ligand binding domain (e.g. MR ligand binding domain) where a ligand can bind. This cavity can be empty, or can contain water molecules or other molecules from the solvent, or can contain ligand atoms. The main binding pocket is the region of space encompassing the residues depicted in FIG. 3 for MR. The binding pocket also includes regions of space near the “main” binding pocket that are not occupied by atoms of NR but that are near the “main” binding pocket, and that are contiguous with the “main” binding pocket.

As used herein, the term “biological activity” means any observable effect flowing from interaction between an NR polypeptide and a ligand. Representative, but non-limiting, examples of biological activity in the context of the subject matter disclosed herein include transcription regulation, ligand binding and peptide binding.

As used herein, the terms “candidate substance” and “candidate compound” are used interchangeably and refer to a substance that is believed to interact with another moiety, for example a given ligand that is believed to interact with a complete, or a fragment of, an NR polypeptide, and which can be subsequently evaluated for such an interaction. Representative candidate substances or compounds include xenobiotics such as drugs and other therapeutic agents, carcinogens and environmental pollutants, natural products and extracts, as well as endobiotics such as mineralocorticosteroids, steroids, fatty acids and prostaglandins. Other examples of candidate compounds that can be investigated using the methods disclosed herein include, but are not restricted to, agonists and antagonists of an NR polypeptide, toxins and venoms, viral epitopes, hormones (e.g., mineralocorticosteroids, opioid peptides, steroids, etc.), hormone receptors, peptides, enzymes, enzyme substrates, co-factors, lectins, sugars, oligonucleotides or nucleic acids, oligosaccharides, proteins, small molecules and monoclonal antibodies.

As used herein, the terms “cells,” “host cells” or “recombinant host cells” are used interchangeably and mean not only the particular subject cell, but also to the progeny or potential progeny of such a cell. Because certain modifications can occur in succeeding generations due to either mutation or environmental influences, such progeny might not, in fact, be identical to the parent cell, but are still included within the scope of the term as used herein.

As used herein, the terms “chimeric protein” or “fusion protein” are used interchangeably and mean a fusion of a first amino acid sequence encoding an NR polypeptide with a second amino acid sequence defining a polypeptide domain foreign to, and not homologous with, any domain of an NR polypeptide. A chimeric protein can include a foreign domain that is found in an organism that also expresses the first protein, or it can be an “interspecies” or “intergenic” fusion of protein structures expressed by different kinds of organisms. In general, a fusion protein can be represented by the general formula X-NR-Y, wherein NR represents a portion of the protein which is derived from an NR polypeptide, and X and Y are independently absent or represent amino acid sequences which are not related to an NR sequence in an organism, which includes naturally occurring mutants. A representative NR is MR.

As used herein, the term “co-activator” means an entity that has the ability to enhance transcription when it is bound to at least one other entity. The association of a co-activator with an entity has the ultimate effect of enhancing the transcription of one or more sequences of DNA. In the context of the presently disclosed subject matter, transcription is preferably nuclear receptor-mediated. By way of specific example, in the presently disclosed subject matter TIF2 (the human analog of mouse glucocorticoid receptor interaction protein 1 (GRIP1)) can bind to a site on the mineralocorticoid receptor, an event that can enhance transcription. TIF2 is therefore a co-activator of MR.

As used herein, the term “co-repressor” means an entity that has the ability to repress transcription when it is bound to at least one other entity. In the context of the subject matter disclosed herein, transcription is preferably nuclear receptor-mediated. The association of a co-repressor with an entity has the ultimate effect of repressing the transcription of one or more sequences of DNA.

As used herein, the term “crystal lattice” means the array of points defined by the vertices of packed unit cells.

As used herein, the term “detecting” means confirming the presence of a target entity by observing the occurrence of a detectable signal, such as a radiologic or spectroscopic signal that will appear exclusively in the presence of the target entity.

As used herein, the term “DNA segment” means a DNA molecule that has been isolated free of total genomic DNA of a particular species. In some embodiments, a DNA segment encoding an MR polypeptide refers to a DNA segment that comprises any of the odd numbered SEQ ID NOs:1-9, but can optionally comprise fewer or additional nucleic acids, yet is isolated away from, or purified free from, total genomic DNA of a source species, such as Homo sapiens. Included within the term “DNA segment” are DNA segments and smaller fragments of such segments, and also recombinant vectors, including, for example, plasmids, cosmids, phages, viruses, and the like.

As used herein, the term “DNA sequence encoding an MR polypeptide” can refer to one or more coding sequences within a particular individual. Moreover, certain differences in nucleotide sequences can exist between individual organisms, which are called alleles. It is possible that such allelic differences might or might not result in differences in amino acid sequence of the encoded polypeptide yet still encode a protein with the same biological activity. As is well known, genes for a particular polypeptide can exist in single or multiple copies within the genome of an individual. Such duplicate genes can be identical or can have certain modifications, including nucleotide substitutions, additions or deletions, all of which still code for polypeptides having substantially the same activity.

As used herein, the phrase “enhancer-promoter” means a composite unit that contains both enhancer and promoter elements. An enhancer-promoter is operatively linked to a coding sequence that encodes at least one gene product.

As used herein, the term “expression” generally refers to the cellular processes by which a biologically active polypeptide is produced.

As used herein, the term “gene” is used for simplicity to refer to a functional protein, polypeptide or peptide encoding unit. As will be understood by those in the art, this functional term includes both genomic sequences and cDNA sequences. Preferred embodiments of genomic and cDNA sequences are disclosed herein.

As used herein, the term “hybridization” means the binding of a probe molecule, a molecule to which a detectable moiety has been bound, to a target sample.

As used herein, the term “interact” means detectable interactions between molecules, such as can be detected using, for example, a yeast two-hybrid assay. The term “interact” is also meant to include “binding” interactions between molecules. Interactions can be, for example, protein-protein or protein-nucleic acid in nature.

As used herein, the term “intron” means a DNA sequence present in a given gene that is not translated into protein.

As used herein, the term “isolated” means oligonucleotides substantially free of other nucleic acids, proteins, lipids, carbohydrates or other materials with which they can be associated, such association being either in cellular material or in a synthesis medium. The term can also be applied to polypeptides, in which case the polypeptide will be substantially free of nucleic acids, carbohydrates, lipids and other undesired polypeptides.

As used herein, the term “labeled” means the attachment of a moiety, capable of detection by spectroscopic, radiologic or other methods, to a probe molecule.

As used herein, the term “mineralocorticoid” means a steroid hormone mineralocorticoid. “Mineralocorticoids” are agonists for the mineralocorticoid receptor. Compounds that mimic mineralocorticoids are also defined as mineralocorticoid receptor agonists. A preferred mineralocorticoid receptor agonist is aldosterone. “Mineralocorticoid” as used herein also includes weakly binding ligands, especially those that bind strongly with an NR other than MR. Other common mineralocorticoid receptor agonists include deoxycorticosterone, as well as those disclosed in the Examples presented herein. As used herein, mineralocorticoid is intended to include, for example, the following generic and brand name mineralocorticoids: spironolactone, ALDACTONE (G.D. Searle LLC, Chicago, Ill., USA), canrenone, eplerenone, and INSPRA (Pfizer, New York, N.Y., USA).

As used herein, the term “mineralocorticoid receptor,” abbreviated herein as “MR,” means the receptor for a steroid hormone mineralocorticoid. A mineralocorticoid receptor is a steroid receptor and, consequently, a nuclear receptor, since steroid receptors are a subfamily of the superfamily of nuclear receptors. The term “MR” means any polypeptide sequence that can be aligned with human MR such that at least 70%, preferably at least 75%, of the amino acids are identical to the corresponding amino acid in the human MR. The term “MR” also encompasses nucleic acid sequences where the corresponding translated protein sequence can be considered to be an MR. The term “MR” includes invertebrate homologs, whether now known or hereafter identified; preferably, MR nucleic acids and polypeptides are isolated from eukaryotic sources. “MR” further includes vertebrate homologs of MR family members, including, but not limited to, mammalian and avian homologs. Representative mammalian homologs of MR family members include, but are not limited to, murine and human homologs. “MR” specifically encompasses all MR isoforms.

As used herein, the terms “MR gene product”, “MR protein”, “MR polypeptide”, and “MR peptide” are used interchangeably and mean peptides having amino acid sequences that are substantially identical to native amino acid sequences from the organism of interest and which are biologically active in that they comprise all or a part of the amino acid sequence of an MR polypeptide, or cross-react with antibodies raised against an MR polypeptide, or retain all or some of the biological activity (e.g., DNA or ligand binding ability and/or transcriptional regulation) of the native amino acid sequence or protein. Such biological activity can include immunogenicity. Representative embodiments are set forth in any of even numbered SEQ ID NOs:2-10. The terms “MR gene product”, “MR protein”, “MR polypeptide”, and “MR peptide” also include analogs of an MR polypeptide. By “analog” is intended that a DNA or peptide sequence can contain alterations relative to the sequences disclosed herein, yet retain all or some of the biological activity of those sequences. Analogs can be derived from genomic nucleotide sequences as are disclosed herein or from other organisms, or can be created synthetically. Those skilled in the art will appreciate that other analogs, as yet undisclosed or undiscovered, can be used to design and/or construct MR analogs. There is no need for an “MR gene product”, “MR protein”, “MR polypeptide”, or “MR peptide” to comprise all or substantially all of the amino acid sequence of an MR polypeptide gene product. Shorter or longer sequences are anticipated to be of use with the presently disclosed subject matter; shorter sequences are herein referred to as “segments”. Thus, the terms “MR gene product”, “MR protein”, “MR polypeptide”, and “MR peptide” also include fusion or recombinant MR polypeptides and proteins comprising sequences disclosed herein. Methods of preparing such proteins are disclosed herein and are known in the art.

As used herein, the terms “MR gene” and “recombinant MR gene” mean a nucleic acid molecule comprising an open reading frame encoding an MR polypeptide of the presently disclosed subject matter, including both exon and (optionally) intron sequences.

As used herein, the term “modified” means an alteration from an entity's normally occurring state. An entity can be modified by removing discrete chemical units or by adding discrete chemical units. The term “modified” encompasses detectable labels as well as those entities added as aids in purification.

As used herein, the term “modulate” means an increase, decrease, or other alteration of any or all chemical and biological activities or properties of a wild-type or mutant NR polypeptide, in some embodiments a wild-type or mutant MR polypeptide. The term “modulation” as used herein refers to both upregulation (i.e., activation or stimulation) and downregulation (i.e. inhibition or suppression) of a response, and includes responses that are upregulated in one cell type or tissue, and down-regulated in another cell type or tissue.

As used herein, the term “molecular replacement” means a method that involves generating a preliminary model of the wild-type NR ligand binding domain, or an NR mutant crystal whose structure coordinates are unknown, by orienting and positioning a molecule or model whose structure coordinates are known (e.g., a nuclear receptor) within the unit cell of the unknown crystal so as best to account for the observed diffraction pattern of the unknown crystal. Phases can then be calculated from this model and combined with the observed amplitudes to give an approximate Fourier synthesis of the structure whose coordinates are unknown. This, in turn, can be subject to any of the several forms of refinement to provide a final, accurate structure of the unknown crystal. See, e.g., Lattman, (1985) Method Enzymol., 115: 55-77; Rossmann, ed, (1972) The Molecular Replacement Method, Gordon & Breach, New York. By way of example, using the structure coordinates of the ligand binding domain of MR provided herein, molecular replacement can be used to determine the structure coordinates of a crystalline mutant or homologue of the MR ligand binding domain, including other NRs, or of a different crystal form of the MR ligand binding domain.

As used herein, the term “mutation” carries its traditional connotation and means a change, inherited, naturally occurring or introduced, in a nucleic acid or polypeptide sequence, and is used in its sense as generally known to those of skill in the art.

As used herein, the term “nuclear receptor”, occasionally abbreviated herein as “NR”, means a member of the superfamily of receptors that comprises at least the subfamilies of steroid receptors, thyroid hormone receptors, retinoic acid receptors and vitamin D receptors. Thus, a given nuclear receptor can be further classified as a member of a subfamily while retaining its status as a nuclear receptor.

As used herein, the phrase “operatively linked” means that an enhancer-promoter is connected to a coding sequence in such a way that the transcription of that coding sequence is controlled and regulated by that enhancer-promoter. Techniques for operatively linking an enhancer-promoter to a coding sequence are well known in the art; the precise orientation and location relative to a coding sequence of interest is dependent, inter alia, upon the specific nature of the enhancer-promoter.

As used herein, the term “partial agonist” means an entity that can bind to a receptor and induce only part of the changes in the receptors that are induced by agonists. The differences can be qualitative or quantitative. Thus, a partial agonist can induce some of the conformation changes induced by agonists, but not others, or it can only induce certain changes to a limited extent.

As used herein, the term “partial antagonist” means an entity that can bind to a receptor and inhibit only part of the changes in the receptors that are induced by antagonists. The differences can be qualitative or quantitative. Thus, a partial antagonist can inhibit some of the conformation changes induced by an antagonist, but not others, or it can inhibit certain changes to a limited extent.

As used herein, the term “polypeptide” means any polymer comprising any of the 20 protein amino acids, regardless of its size. Although “protein” is often used in reference to relatively large polypeptides, and “peptide” is often used in reference to small polypeptides, usage of these terms in the art overlaps and varies. The term “polypeptide” as used herein refers to peptides, polypeptides and proteins, unless otherwise noted. As used herein, the terms “protein”, “polypeptide” and “peptide” are used interchangeably herein when referring to a gene product.

As used herein, the term “primer” means a sequence comprising two or more deoxyribonucleotides or ribonucleotides, preferably more than three, and more preferably more than eight and most preferably at least about 20 nucleotides of an exonic or intronic region. Such oligonucleotides are preferably between ten and thirty bases in length.

As used herein, the term “sequencing” means determining the ordered linear sequence of nucleic acids or amino acids of a DNA or protein target sample, using conventional manual or automated laboratory techniques.

As used herein, the term “space group” means the arrangement of symmetry elements of a crystal.

As used herein, the term “steroid receptor” means a nuclear receptor that can bind or associate with a steroid compound. Steroid receptors are a subfamily of the superfamily of nuclear receptors. The subfamily of steroid receptors comprises mineralocorticoid receptors and, therefore, a mineralocorticoid receptor is a member of the subfamily of steroid receptors and the superfamily of nuclear receptors.

As used herein, the terms “structure coordinates” and “structural coordinates” mean mathematical coordinates derived from mathematical equations related to the patterns obtained on diffraction of a monochromatic beam of X-rays by the atoms (scattering centers) of a molecule in crystal form. The diffraction data are used to calculate an electron density map of the repeating unit of the crystal. The electron density maps are used to establish the positions of the individual atoms within the unit cell of the crystal.

Those of skill in the art understand that a set of coordinates determined by X-ray crystallography is not without standard error. In general, the error in the coordinates tends to be reduced as the resolution is increased, since more experimental diffraction data is available for the model fitting and refinement. Thus, for example, more diffraction data can be collected from a crystal that diffracts to a resolution of 2.8 angstroms than from a crystal that diffracts to a lower resolution, such as 3.5 angstroms. Consequently, the refined structural coordinates will usually be more accurate when fitted and refined using data from a crystal that diffracts to higher resolution. The design of ligands and modulators for MR or any other NR depends on the accuracy of the structural coordinates. If the coordinates are not sufficiently accurate, then the design process will be ineffective. In most cases, it is very difficult or impossible to collect sufficient diffraction data to define atomic coordinates precisely when the crystals diffract to a resolution of only 3.5 angstroms or poorer. Thus, in most cases, it is difficult to use X-ray structures in structure-based ligand design when the X-ray structures are based on crystals that diffract to a resolution of only 3.5 angstroms or poorer. However, common experience has shown that crystals diffracting to 2.8 angstroms or better can yield X-ray structures with sufficient accuracy to greatly facilitate structure-based drug design. Further improvement in the resolution can further facilitate structure-based design, but the coordinates obtained at 2.8 angstroms resolution are generally adequate for most purposes.

Also, those of skill in the art will understand that NR proteins can adopt different conformations when different ligands are bound. In particular, NR proteins will adopt substantially different conformations when agonists and antagonists are bound. Subtle variations in the conformation can also occur when different agonists are bound, and when different antagonists are bound. Generally, structure-based design of NR modulators depends to some degree on knowledge of the differences in conformation that occur when agonists and antagonists are bound. Thus, structure-based modulator design is most facilitated by the availability of X-ray structures of complexes with potent agonists as well as potent antagonists.

As used herein, the term “substantially pure” means that the polynucleotide or polypeptide is substantially free of the sequences and molecules with which it is associated in its natural state, and those molecules used in the isolation procedure. The term “substantially free” means that the sample is at least 50%, preferably at least 70%, more preferably 80% and most preferably 90% free of the materials and compounds with which is it associated in nature.

As used herein, the term “target cell” refers to a cell, into which it is desired to insert a nucleic acid sequence or polypeptide, or to otherwise effect a modification from conditions known to be standard in the unmodified cell. A nucleic acid sequence introduced into a target cell can be of variable length. Additionally, a nucleic acid sequence can enter a target cell as a component of a plasmid or other vector or as a naked sequence.

As used herein, the term “transcription” means a cellular process involving the interaction of an RNA polymerase with a gene that directs the expression as RNA of the structural information present in the coding sequences of the gene. The process includes, but is not limited to the following steps: (a) the transcription initiation, (b) transcript elongation, (c) transcript splicing, (d) transcript capping, (e) transcript termination, (f) transcript polyadenylation, (g) nuclear export of the transcript, (h) transcript editing, and (i) stabilizing the transcript.

As used herein, the term “transcription factor” means a cytoplasmic or nuclear protein which binds to such gene, or binds to an RNA transcript of such gene, or binds to another protein which binds to such gene or such RNA transcript or another protein which in turn binds to such gene or such RNA transcript, so as to thereby modulate expression of the gene. Such modulation can additionally be achieved by other mechanisms; the essence of “transcription factor for a gene” is that the level of transcription of the gene is altered in some way.

As used herein, the term “unit cell” means a basic parallelipiped shaped block. Regular assembly of such blocks can construct the entire volume of a crystal. Each unit cell comprises a complete representation of the unit of pattern, the repetition of which builds up the crystal. Thus, the term “unit cell” means the fundamental portion of a crystal structure that is repeated infinitely by translation in three dimensions. A unit cell is characterized by three vectors a, b, and c, not located in one plane, which form the edges of a parallelepiped. Angles α, β and γ define the angles between the vectors: angle α is the angle between vectors b and c; angle β is the angle between vectors a and c; and angle γ is the angle between vectors a and b. The entire volume of a crystal can be constructed by regular assembly of unit cells; each unit cell comprises a complete representation of the unit of pattern, the repetition of which builds up the crystal.

As used herein, the terms “weakly binding ligand” and “weak ligand” are used interchangeably and mean a ligand that binds a receptor with low affinity. The binding strength of a ligand to a receptor can be experimentally measured using any of several procedures, as is generally known in the art. In some embodiments of the presently disclosed subject matter, ligand binding is measured using a competitive binding assay in which a ligand of interest competes with a labeled ligand for binding to a receptor. The amount of ligand of interest required to reduce binding of the labeled ligand to the receptor (e.g., as measured by detection of the label bound to the receptor) by 50% is a quantitative measurement of the affinity of the ligand of interest for the particular receptor. This quantitative measurement can be expressed as an IC50 value. Therefore, the greater the IC50 value of a ligand, the lower the binding affinity the ligand has for the receptor. In some embodiments, ligands having an IC50>50 nM for a receptor are considered weakly binding ligands for the receptor.

II. Description of Tables

Table 1 summarizes data of binding affinities for various ligands with wild-type MR and mutant MRs (MR C808S; MR S810L; and MR C808S, S810L).

Table 2 lists mutations of the MR LBD (712-984) gene for testing solution solubility and stability.

Table 3 is a chart of sequence identity between the ligand binding domains of several nuclear receptors.

Tables 4 and 5 summarize the crystal and data statistics obtained from the crystallized ligand binding domain of MR LBD that was co-crystallized with deoxycorticosterone only, or with a fragment of the co-activator TIF2. Also included is data of crystallized mutant MR molecules in combination with deoxycorticosterone, deoxycorticosterone+TIF2, aldosterone, progesterone, spirinolactone and cortisone. Data on the unit cell are presented, including data on the crystal space group, unit cell dimensions, molecules per asymmetric cell and crystal resolution.

Table 6 is a table of the atomic structure coordinate data obtained from X-ray diffraction from the ligand binding domain of native MR (residues 727-984) in complex with deoxycorticosterone.

Table 7 is a table of the atomic structure coordinate data obtained from X-ray diffraction from the ligand binding domain of MR C808S (residues 727-984) in complex with deoxycorticosterone.

Table 8 is a table of the atomic structure coordinate data obtained from X-ray diffraction from the ligand binding domain of MR C808S (residues 727-984) in complex with deoxycorticosterone and a peptide containing residues 732-756 from the TIF2 co-activator.

Table 9 is a table of the atomic structure coordinate data obtained from X-ray diffraction from the ligand binding domain of MR C808S (residues 727-984) in complex with aldosterone.

Table 10 is a table of the atomic structure coordinate data obtained from X-ray diffraction from the ligand binding domain of MR C808S (residues 727-984) in complex with progesterone.

Table 11 is a table of the atomic structure coordinate data obtained from X-ray diffraction from the ligand binding domain of MR C808S/S810L (residues 727-984) in complex with progesterone.

Table 12 is a table of the atomic structure coordinate data obtained from X-ray diffraction from the ligand binding domain of MR C808S/S810L (residues 727-984) in complex with spironolactone.

Table 13 is a table of the atomic structure coordinate data obtained from X-ray diffraction from the ligand binding domain of MR C808S/S810L (residues 727-984) in complex with cortisone.

III. General Considerations

The present disclosure will usually be applicable mutatis mutandis to nuclear receptors in general, in some embodiments to steroid receptors and in some embodiments to mineralocorticoid receptors, including MR isoforms, as discussed herein, based, in part, on the patterns of nuclear receptor and steroid receptor structure and modulation that have emerged as a consequence of the present disclosure.

The nuclear receptor superfamily has been subdivided into two subfamilies: the GR subfamily (also referred to as the steroid receptors and denoted SRs), comprising GR, AR (androgen receptor), MR (mineralocorticoid receptor) and PR (progesterone receptor) and the thyroid hormone receptor (TR) subfamily, comprising TR, vitamin D receptor (VDR), retinoic acid receptor (RAR), retinoid X receptor (RXR), and most orphan receptors. This division has been made on the basis of DNA binding domain structures, interactions with heat shock proteins (HSP), and ability to form dimers.

The mineralocorticoid receptor is a steroid receptor and thus a member of the superfamily of nuclear receptors and the subfamily of steroid receptors. The human mineralocorticoid receptor comprises 984 amino acids and has three major functional domains. From amino to carboxyl terminal end, these functional domains include the constitutive transcriptional activation function AF1, a DNA binding domain, and a ligand binding domain in succession. The AF1 domain spans amino acid positions 1-600 and regulates gene activation. The DNA binding domain is from amino acid positions 601 to 674 and has nine cysteine residues, eight of which are organized in the form of two zinc fingers analogous to Xenopus transcription factor IIIA. The DNA binding domain binds to the regulatory sequences of genes that are induced or deinduced by mineralocorticoids. Amino acids 712 to 984 form the LBD, which binds mineralocorticoid to activate the receptor. This region of the receptor also has the nuclear localization signal. Despite the aforementioned indirect characterization of the structure of MR, until the present disclosure, a detailed three-dimensional model of the ligand binding domain of MR had not been achieved.

MR forms a heteromultimeric cytoplasmic complex with heat shock protein(s) (HSP) in the absence of ligand. After ligand binding, MR dissociates of HSP, and translocates to the nucleus and binds to DNA as a homodimer. It also can bind the coactivators NCOA1, TIF1 and NRIP1.

Most members of the superfamily, including orphan receptors, possess at least two transcription activation subdomains, one of which is constitutive and resides in the amino terminal domain (AF-1), and the other of which (AF-2) resides in the ligand binding domain, whose activity is regulated by binding of an agonist ligand. The function of AF-2 requires an activation domain (also called transactivation domain) that is highly conserved among the receptor superfamily. Most LBDs contain an activation domain. Some mutations in this domain abolish AF-2 function, but leave ligand binding and other functions unaffected. Ligand binding allows the activation domain to serve as an interaction site for essential co-activator proteins that function to stimulate (or in some cases, inhibit) transcription.

Analysis and alignment of amino acid sequences, and X-ray and NMR structure determinations, have shown that nuclear receptors have a modular architecture with three main domains, which matches MR as discussed above:

1) a variable amino-terminal domain (constitutive transcriptional activation function);

2) a highly conserved DNA-binding domain (DBD); and

3) a less conserved carboxy-terminal ligand binding domain (LBD).

In addition, nuclear receptors can have linker segments of variable length between these major domains. Sequence analysis and X-ray crystallography, as disclosed herein, have confirmed that MR also has the same general modular architecture, with the same three domains. The function of MR in human cells presumably requires all three domains in a single amino acid sequence. However, the modularity of MR permits different domains of each protein to separately accomplish certain functions. Some of the functions of a domain within the full-length receptor are preserved when that particular domain is isolated from the remainder of the protein. Using conventional protein chemistry techniques, a modular domain can sometimes be separated from the parent protein. Using conventional molecular biology techniques, each domain can usually be separately expressed with its original function intact or, as discussed herein below, chimeras comprising two different proteins can be constructed, wherein the chimeras retain the properties of the individual functional domains of the respective nuclear receptors from which the chimeras were generated.

The carboxy-terminal activation subdomain, is in close three-dimensional proximity in the LBD to the ligand, so as to allow for ligands bound to the LBD to coordinate (or interact) with amino acid(s) in the activation subdomain. As described herein, the LBD of a nuclear receptor can be expressed, crystallized, its three dimensional structure determined with a ligand bound (either using crystal data from the same receptor or a different receptor or a combination thereof), and computational methods used to design ligands to its LBD, particularly ligands that contain an extension moiety that coordinates the activation domain of the nuclear receptor.

The LBD is the second most highly conserved domain in these receptors. As its name suggests, the LBD binds ligands. With many nuclear receptors, including MR, binding of the ligand can induce a conformational change in the LBD that can, in turn, activate transcription of certain target genes. Whereas integrity of several different LBD sub-domains is important for ligand binding, truncated molecules containing only the LBD retain normal ligand-binding activity. This domain also participates in other functions, including dimerization, nuclear translocation and transcriptional activation, as described herein.

Nuclear receptors usually have HSP binding domains that present a region for binding to the LBD and can be modulated by the binding of a ligand to the LBD. For many of the nuclear receptors ligand binding induces a dissociation of heat shock proteins such that the receptors can form dimers in most cases, after which the receptors bind to DNA and regulate transcription. Consequently, a ligand that stabilizes the binding or contact of the heat shock protein binding domain with the LBD can be designed using the computational methods described herein.

With the receptors that are associated with the HSP in the absence of the ligand, dissociation of the HSP results in dimerization of the receptors. Dimerization is due to receptor domains in both the DBD and the LBD. Although the main stimulus for dimerization is dissociation of the HSP, the ligand-induced conformational changes in the receptors can have an additional facilitative influence. With the receptors that are not associated with HSP in the absence of the ligand, particularly with the TR, ligand binding can affect the pattern of dimerization. The influence depends on the DNA binding site context, and can also depend on the promoter context with respect to other proteins that can interact with the receptors. A common pattern is to discourage monomer formation, with a resulting preference for heterodimer formation over dimer formation on DNA.

Nuclear receptor LBDs usually have dimerization domains that present a region for binding to another nuclear receptor and can be modulated by the binding of a ligand to the LBD. Consequently, a ligand that disrupts the binding or contact of the dimerization domain can be designed using the computational methods described herein to produce a partial agonist or antagonist.

The amino terminal domain of MR is the least conserved of the three domains. This domain is involved in transcriptional activation and, its uniqueness might dictate selective receptor-DNA binding and activation of target genes by MR subtypes. This domain can display synergistic and antagonistic interactions with the domains of the LBD.

The DNA binding domain has the most highly conserved amino acid sequence amongst the GRs, including MR. It typically comprises about 70 amino acids that fold into two zinc finger motifs, wherein a zinc atom coordinates four cysteines. The DBD comprises two perpendicularly oriented α-helixes that extend from the base of the first and second zinc fingers. The two zinc fingers function in concert along with non-zinc finger residues to direct the MR to specific target sites on DNA and to align receptor dimer interfaces. Various amino acids in the DBD influence spacing between two half-sites (which usually comprises six nucleotides) for receptor dimerization. The optimal spacings facilitate cooperative interactions between DBDs, and D box residues are part of the dimerization interface. Other regions of the DBD facilitate DNA-protein and protein-protein interactions are involved in dimerization.

In nuclear receptors that bind to an HSP, the ligand-induced dissociation of HSP with consequent dimer formation allows, and therefore, promotes DNA binding. With receptors that are not associated (as in the absence of ligand), ligand binding tends to stimulate DNA binding of heterodimers and dimers, and to discourage monomer binding to DNA. However, with DNA containing only a single half site, the ligand tends to stimulate the receptor's binding to DNA. The effects are modest and depend on the nature of the DNA site and probably on the presence of other proteins that can interact with the receptors. Nuclear receptors usually have DNA binding domains (DBD) that present a region for binding to DNA and this binding can be modulated by the binding of a ligand to the LBD.

The modularity of the members of the nuclear receptor superfamily permits different domains of each protein to separately accomplish different functions, although the domains can influence each other. The separate function of a domain is usually preserved when a particular domain is isolated from the remainder of the protein. Using conventional protein chemistry techniques a modular domain can sometimes be separated from the parent protein. By employing conventional molecular biology techniques each domain can usually be separately expressed with its original function intact or chimerics of two different nuclear receptors can be constructed, wherein the chimerics retain the properties of the individual functional domains of the respective nuclear receptors from which the chimerics were generated.

Various structures have indicated that most nuclear receptor LBDs adopt the same general folding pattern. This fold consists of 10-12 alpha helices arranged in a bundle, together with several beta-strands, and linking segments. A preferred MR LBD structure of the present disclosure has 12 helices. Structural studies have shown that most of the alpha-helices and beta-strands have the same general position and orientation in all nuclear receptor structures, whether ligand is bound or not. However, the AF2 helix has been found in different positions and orientations relative to the main bundle, depending on the presence or absence of the ligand, and also on the chemical nature of the ligand. These structural studies have suggested that many nuclear receptors share a common mechanism of activation, where binding of activating ligands helps to stabilize the AF2 helix in a position and orientation adjacent to helices-3, -4, and -10, covering an opening to the ligand binding site. This position and orientation of the AF2 helix, which will be called the “active conformation”, creates a binding site for co-activators. See, e.g., Nolte et al., (1998) Nature 395:137-43; and Shiau et al., (1998) Cell 95: 927-37. This co-activator binding site has a central lipophilic pocket that can accommodate leucine side-chains from co-activators, as well as a “charge-clamp” structure consisting essentially of a lysine residue from helix-3 and a glutamic acid residue from the AF2 helix.

Structural studies have shown that co-activator peptides containing the sequence LXXLL (where L is leucine and X can be a different amino acid in different cases) can bind to this co-activator binding site by making interactions with the charge clamp lysine and glutamic acid residues, as well as the central lipophilic region. This co-activator binding site is disrupted when the AF2 helix is shifted into other positions and orientations. In PPARγ, activating ligands such as rosiglitazone (BRL49653) make a hydrogen bonding interaction with tyrosine-473 in the AF2 helix. Nolte et al., (1998) Nature 395:137-43; Gampe et al., (2000) Mol. Cell 5: 545-55. Similarly, in GR, the dexamethasone ligand makes van der Waals interaction with the side chain of leucine-753 from the AF2 helix. This interaction is believed in part to stabilize the AF2 helix in the active conformation, thereby allowing co-activators to bind and thus activating transcription from target genes.

With certain antagonist ligands, or in the absence of any ligand, the AF2 helix can be held less tightly in the active conformation, or can be free to adopt other conformations. This would either destabilize or disrupt the co-activator binding site, thereby reducing or eliminating co-activator binding and transcription from certain target genes. Some of the functions of the MR protein depend on having the full-length amino acid sequence and certain partner molecules, such as co-activators and DNA. However, other functions, including ligand binding and ligand-dependent conformational changes, can be observed experimentally using isolated domains, chimeras and mutant molecules.

As described herein, the LBD of an MR can be mutated or engineered, expressed, crystallized, its three dimensional structure determined with a ligand bound as disclosed herein, and computational methods can be used to design ligands to nuclear receptors, in some embodiments to steroid receptors, and in some embodiments to mineralocorticoid receptors.

IV. Modeling Molecular Interactions of Low Affinity Ligands With an NR LBD Using a Surrogate NR LBD

There is a need to analyze and understand the molecular interactions of NRs with low affinity, or weakly binding ligands (e.g. having a binding affinity IC50>50 nM). This information would be particularly useful in determining the orientation of novel, weak, non-steroidal ligands in an NR. For example, such information is applicable to determining how weakly binding ligands, such as cross-reactive drugs, affect NR function. Unfortunately, until the present disclosure it has proven very difficult to obtain adequate expression levels of NR LBDs in combination with weakly binding ligands for crystallography purposes. As such, crystallographic determination of a nuclear receptor, including particularly a steroid receptor, with a weakly binding ligand has heretofore not been achieved. Disclosed below are methods for obtaining crystal structures of NR LBDs in combination with weakly binding ligands in order to study the molecular interactions between the NR LBD and the ligand.

Mutation of select residues of an NR can facilitate greater ease of expression and crystallization of the NR by, for example, increasing the solubility of the protein or improving ligand binding properties. The selected mutations confer to the mutated NR the ability to bind low affinity ligands and partial agonists while remaining soluble, thereby effectively converting weakly binding ligands into strong ligands with regard to the mutated NR. Residues are selected for mutation that do not alter significantly the structure of the protein or its interactions with a ligand. To illustrate the effect select receptor mutations can have on ligand binding affinities, Table 1 provides data showing the IC50 binding affinities, as measured by competitive binding assay, of a number of different ligands for wild-type and selectively mutated MR. Generally, the mutated MRs have an increased binding affinity for many ligands as compared to wild-type MR. In particular, weakly binding ligands, such as cortisone, will bind with strong affinity to mutated MR, e.g. MR C808S, S810L (IC50=9 nM), while only very weakly binding wild-type MR (IC50=>10 μM). As Table 1 illustrates, the subject matter disclosed herein permits the study of weak ligands bound with receptors, such as MR and cortisone, where it was difficult or impossible to do so prior to the presently disclosed subject matter.

TABLE 1
Ligand Binding Affinities for Wild-Type and
Mutant MRs on MR Constructs

| | | | | | | | | MR |
|---|---|---|---|---|---|---|---|---|
| | Reporter | | Wild type | | MR | | MR | C808S |
| | Alone | | MR | | C808S | | S810L | S810L |
| Aldosterone | >10 | uM | 0.2 | nM | 0.099 | nM | 0.18 nM | 0.15 nM |
| Cortisol | 785 | nM | 21 | nM | 30 | nM | 20 nM | 21 nM |
| Cortisone | >10 | uM | >10 | uM | >10 | uM | 20 nM | 9 nM |
| Deoxycor- | >10 | uM | 0.17 | nM | 0.085 | nM | 0.60 nM | 0.67 nM |
| ticosterone | | | | | | | | |
| Dexa- | 16 | nM | 11 | nM | 5 | nM | 3 nM | 3 nM |
| methasone | | | | | | | | |
| Progesterone | >10 | uM | 25 | nM | 513 | nM | 1.8 nM | 0.6 nM |
| Spirono- | 3.6 | nM | 1.2 | nM | 62.5 | nM | 0.4 nM | 0.6 nM |
| lactone | | | | | | | | |

One method useful with the presently disclosed subject matter is to selectively mutate one or more native residues to residues that provide these desirable properties. Upon a review of the present disclosure one of ordinary skill in the art will recognize the utility of strategies for mutating residues to increase protein solubility, ligand binding and/or crystallization properties. These strategies are intended to be included with the presently disclosed subject matter.

Using mutagenesis and expression analysis, an expression construct of an MR that has one or more of the desired properties listed above has been identified. In addition, multiple crystal structures of the native and mutant receptor reveal the overall binding characteristics of ligands are conserved. Using mutated forms of an MR LBD, as described herein, a co-crystal of the mutant MR LBD and a weakly binding ligand can be formed. In one application of the presently disclosed subject matter, this allows the mutant MR LBD to serve as a surrogate for the native receptors of these ligands and allows for the determination of crystal structures of the mutant MR bound to the other receptor's native ligands. In another application of the presently disclosed subject matter, the mutated MR LBD can serve as a surrogate ligand for novel, weakly binding non-steroidal ligands, which cannot be obtained with the AR, PR and GR constructs currently in existence.

In accordance with the above disclosure, a method for modeling one or more molecular interactions of a native NR with a ligand having low affinity for the native NR is provided. The method comprises:

(a) crystallizing a surrogate ligand binding domain polypeptide in complex with a ligand having low affinity for a native NR to form a crystallized surrogate ligand binding domain polypeptide-ligand complex, wherein the surrogate ligand binding domain polypeptide comprises at least one mutation, and wherein the mutation improves ligand binding, crystal forming properties, or both ligand binding and crystal forming properties; and

(b) analyzing the crystallized complex to determine a three-dimensional structure of the crystallized complex, whereby the three-dimensional structure of the crystallized complex models one or more molecular interactions of the native NR with the ligand.

In some embodiments, the surrogate LBD polypeptide is a mutated MR LBD polypeptide and the native NR is an SR, optionally the SR is AR, PR, MR or GR. Further, in some embodiments the mutated MR LBD polypeptide has mutations of C808S (SEQ ID NO:6), S810L (SEQ ID NO:8), or both C808S and S810L (SEQ ID NO:10).

The low affinity (weakly binding, with a binding affinity IC50>50 nM) ligand in some embodiments is either a steroid or non-steroid. As non-limiting examples, the ligand can be aldosterone, deoxycorticosterone, progesterone, spironolactone or cortisone.

In some embodiments, the crystallized complex comprises the surrogate LBD polypeptide, the ligand, and either a co-activator or a co-repressor polypeptide, or a fragment of a co-activator or a co-repressor polypeptide. As a non-limiting example, the polypeptide in complex with the surrogate LBD polypeptide and the ligand can be a fragment of the TIF2 co-activator polypeptide (SEQ ID NO:11).

V. The Deoxvcorticosterone Ligand

Ligand binding can induce transcriptional activation functions in a variety of ways. One way is through the dissociation of the HSP from receptors. This dissociation, with consequent dimerization of the receptors and their binding to DNA or other proteins in the nuclear chromatin, allows transcriptional regulatory properties of the receptors to be manifest. This can be especially true of such functions on the amino terminus of the receptors.

Another way to alter the receptor is to interact it with other proteins involved in transcription. These could be proteins that interact directly or indirectly with elements of the proximal promoter or proteins of the proximal promoter. Alternatively, the interactions can be through other transcription factors that themselves interact directly or indirectly with proteins of the proximal promoter. Several different proteins have been described that bind to the receptors in a ligand-dependent manner. In addition, it is possible that in some cases, the ligand-induced conformational changes do not affect the binding of other proteins to the receptor, but do affect their abilities to regulate transcription.

In one aspect of the presently disclosed subject matter, an MR LBD was co-crystallized with a fragment of the co-activator TIF2 and the ligand deoxycorticosterone. Deoxycorticosterone has the IUPAC name 21-hydroxypregn-4-ene-3,20-dione. It has a molecular weight of 330.5. The empirical formula for deoxycorticosterone is C₂₁H₃₀O₃.

The cortex of the adrenal gland secretes deoxycorticosterone. It has about three percent of the sodium-retaining activity of aldosterone. The acetate and pivalate salts are used for mineralocorticoid replacement therapy.

VI. The TIF2 Fragment

The nuclear receptor co-activator TIF2 (SEQ ID NO:11) was co-crystallized in one aspect of the presently disclosed subject matter. Structurally, the nuclear receptor coactivator TIF2 comprises one domain that reacts with a nuclear receptor (nuclear receptor interaction domain, abbreviated “NID”) and two autonomous activation domains, AD1 and AD2 (Voeael et al., (1998) EMBO J. 17: 507-519). The TIF2 NID comprises three NR-interacting modules, with each module comprising the motif, LXXLL (SEQ ID NO:12) (Voegel et al., (1998) EMBO J. 17: 507-519). Mutation of the motif abrogates TIF2's ability to interact with the ligand-induced activation function-2 (AF-2) found in the ligand-binding domains (LBDs) of many NRs. Presently, it is thought that TIF2 AD1 activity is mediated by CREB binding protein (CBP), however, TIF2 AD2 activity does not appear to involve interaction with CBP (Voeqel et al., (1998) EMBO J. 17: 507-519).

As disclosed herein, residues 732-756 of the TIF2 protein (SEQ ID NO:11) were co-crystallized with MR and deoxycorticosterone. These residues comprise the LXXLL (SEQ ID NO:12) of AD-2, the third motif in the linear sequence of TIF2. The TIF2 fragment is 25 residues in length and was synthesized using an automated peptide synthesis apparatus. SEQ ID NO:11, and other sequences corresponding to TIF2 and other co-activators and co-repressors, can be similarly synthesized using automated apparatuses.

VII. Design, Preparation and Structural Analysis of MR Polvleptides and MR LBD Mutants and Structural Equivalents

The presently disclosed subject matter provides for the generation of MR polypeptides and MR mutants (preferably MR LBD mutants), and the ability to solve the crystal structures of those that crystallize. In a preferred embodiment, the presently disclosed subject matter provides for the first time for the expression of a soluble MR polypeptide in bacteria, more preferably, in E. coli. Indeed, MR LBDs having one or more point mutations were crystallized and solved in one aspect of the subject matter disclosed herein. Thus, an aspect of the presently disclosed subject matter involves the use of both targeted and random mutagenesis of the MR gene for the production of a recombinant protein with improved or desired characteristics for the purpose of crystallization, characterization of biologically relevant protein-protein interactions including interactions with weak ligands, and compound screening assays, or for the production of a recombinant protein having other desirable characteristic(s). Polypeptide products produced by the methods of the present subject matter are also disclosed herein.

The structure coordinates of an NR, SR or MR LBD provided in accordance with the present subject matter also facilitate the identification of related proteins or enzymes analogous to MR in function, structure or both, which can lead to novel therapeutic modes for treating or preventing a range of disease states. More particularly, through the provision of the mutagenesis approaches as well as the three-dimensional structure of an MR LBD disclosed herein, desirable sites for mutation are identified.

VII.A. MR Polypeptides

The generation of chimeric MR polypeptides is also an aspect of the presently disclosed subject matter. Such a chimeric polypeptide can comprise an MR LBD polypeptide or a portion of an MR LBD that is fused to a candidate polypeptide or a suitable region of the candidate polypeptide. Throughout the present disclosure it is intended that the term “mutant” encompass not only mutants of an MR LBD polypeptide but chimeric proteins generated using an MR LBD as well. It is thus intended that the following discussion of mutant MR LBDs apply mutatis mutandis to chimeric MR polypeptides and MR LBD polypeptides and to structural equivalents thereof.

In accordance with the subject matter disclosed herein, a mutation can be directed to a particular site or combination of sites of a wild-type MR LBD. For example, an accessory binding site or the binding pocket can be chosen for mutagenesis. Similarly, a residue having a location on, at or near the surface of the polypeptide can be replaced, resulting in an altered surface charge of one or more charge units, as compared to the wild-type MR and MR LBDs. Alternatively, an amino acid residue in an MR or an MR LBD can be chosen for replacement based on its hydrophilic or hydrophobic characteristics.

Such mutants can be characterized by any one of several different properties, i.e. a “desired” or “predetermined” characteristic as compared with the wild type MR LBD. For example, such mutants can have an altered surface charge of one or more charge units, or can have an increase in overall stability. Other mutants can have altered ligand specificity in comparison with, or a higher specific activity than, a wild-type MR or an MR LBD, for example, with respect to weak ligands.

MR and MR LBD mutants disclosed herein can be generated in a number of ways. For example, the wild-type sequence of an MR or an MR LBD can be mutated at those sites identified using methods disclosed herein as desirable for mutation, by means of oligonucleotide-directed mutagenesis or other conventional methods, such as deletion. Alternatively, mutants of an MR or an MR LBD can be generated by the site-specific replacement of a particular amino acid with an unnaturally occurring amino acid. In addition, MR or MR LBD mutants can be generated through replacement of an amino acid residue, for example, a particular cysteine or methionine residue, with selenocysteine or selenomethionine. This can be achieved by growing a host organism capable of expressing either the wild-type or mutant polypeptide on a growth medium depleted of either natural cysteine or methionine (or both) but enriched in selenocysteine or selenomethionine (or both).

As disclosed in the Examples presented below, mutations can be introduced into a DNA sequence coding for an MR or an MR LBD using synthetic oligonucleotides. These oligonucleotides contain nucleotide sequences flanking the desired mutation sites. Mutations can be generated in the full-length DNA sequence of an MR or an MR LBD or in any sequence coding for polypeptide fragments of an MR or an MR LBD.

According to the presently disclosed subject matter, a mutated MR or MR LBD DNA sequence produced by the methods described above, or any alternative methods known in the art, can be expressed using an expression vector. An expression vector, as is well known to those of skill in the art, typically includes elements that permit autonomous replication in a host cell independent of the host genome, and one or more phenotypic markers for selection purposes. Either prior to or after insertion of the DNA sequences surrounding the desired MR or MR LBD mutant coding sequence, an expression vector also will include control sequences encoding a promoter, operator, ribosome binding site, translation initiation signal, and, optionally, a repressor gene or various activator genes and a signal for termination. In some embodiments, where secretion of the produced mutant is desired, nucleotides encoding a “signal sequence” can be inserted prior to an MR or an MR LBD mutant coding sequence. For expression under the direction of the control sequences, a desired DNA sequence must be operatively linked to the control sequences; that is, the sequence must have an appropriate start signal in front of the DNA sequence encoding the MR or MR LBD mutant, and the correct reading frame to permit expression of that sequence under the control of the control sequences and production of the desired product encoded by that MR or MR LBD sequence must be maintained.

After a review of the disclosure of the subject matter presented herein, any of a wide variety of available expression vectors can be useful to express a mutated coding sequence disclosed herein. These include for example, vectors consisting of segments of chromosomal, non-chromosomal and synthetic DNA sequences, such as various known derivatives of SV40, known bacterial plasmids, e.g., plasmids from E. coli including col E1, pCR1, pBR322, pMB9 and their derivatives, wider host range plasmids, e.g., RP4, phage DNAs, e.g., the numerous derivatives of phage X, e.g., NM 989, and other DNA phages, e.g., M13 and filamentous single stranded DNA phages, yeast plasmids and vectors derived from combinations of plasmids and phage DNAs, such as plasmids which have been modified to employ phage DNA or other expression control sequences. In the preferred embodiments, vectors amenable to expression in a pET-based expression system are employed. The pET expression system is available from Novagen/Invitrogen, Inc., Carlsbad, California. Expression and screening of a polypeptide of the present subject matter in bacteria, preferably E. coli, is an aspect of the presently disclosed subject matter.

In addition, any of a wide variety of expression control sequences-sequences that control the expression of a DNA sequence when operatively linked to it can be used in these vectors to express the mutated DNA sequences according to the subject matter disclosed herein. Such useful expression control sequences, include, for example, the early and late promoters of SV40 for animal cells, the lac system, the trp system the TAC or TRC system, the major operator and promoter regions of phage X, the control regions of fd coat protein, all for E. coli, the promoter for 3-phosphoglycerate kinase or other glycolytic enzymes, the promoters of acid phosphatase, e.g., Pho5, the promoters of the yeast α-mating factors for yeast, and other sequences known to control the expression of genes of prokaryotic or eukaryotic cells or their viruses, and various combinations thereof.

A wide variety of hosts are also useful for producing mutated MR and MR LBD polypeptides according to the subject matter disclosed herein. These hosts include, for example, bacteria, such as E. coli, Bacillus sp. and Streptomyces sp., fungi, such as yeasts, and animal cells, such as CHO and COS-1 cells, plant cells, insect cells, such as SF9 cells, and transgenic host cells. Expression and screening of a polypeptide of the presently disclosed subject matter in bacteria, preferably E. coli, is a preferred aspect of the present subject matter.

It should be understood that not all expression vectors and expression systems function in the same way to express mutated DNA sequences, and to produce modified MR and MR LBD polypeptides or MR or MR LBD mutants. Neither do all hosts function equally well with the same expression system. One of skill in the art can, however, make a selection among these vectors, expression control sequences and hosts without undue experimentation and without departing from the scope of the subject matter disclosed herein. For example, an important consideration in selecting a vector will be the ability of the vector to replicate in a given host. The copy number of the vector, the ability to control that copy number, and the expression of any other proteins encoded by the vector, such as antibiotic markers, should also be considered.

In selecting an expression control sequence, a variety of factors should also be considered. These include, for example, the relative strength of the system, its controllability and its compatibility with the DNA sequence encoding a modified MR or MR LBD polypeptide of the subject matter disclosed herein, with particular regard to the formation of potential secondary and tertiary structures.

Hosts should be selected by consideration of their compatibility with the chosen vector, the toxicity of a modified polypeptide to them, their ability to express mature products, their ability to fold proteins correctly, their fermentation requirements, the ease of purification of a modified MR or MR LBD and safety. Within these parameters, one of skill in the art can select various vector/expression control system/host combinations that will produce useful amounts of a mutant polypeptide. A mutant polypeptide produced in these systems can be purified, for example, via the approaches disclosed in the Examples.

Once a mutation(s) has been generated in the desired location, such as an active site or dimerization site, the mutants can be tested for any one of several properties of interest, i.e. “desired” or “predetermined” positions. For example, mutants can be screened for an altered charge at physiological pH. This property can be determined by measuring the mutant polypeptide isoelectric point (pi) and comparing the observed value with that of the wild-type parent. Isoelectric point can be measured by gel-electrophoresis according to the method of Wellner (Wellner, (1971) Anal. Chem. 43: 597). A mutant polypeptide containing a replacement amino acid located at the surface of the enzyme, as provided by the structural information disclosed herein, can lead to an altered surface charge and an altered pl.

VII.B. Generation of Engineered MR or MR LBD Mutants

In another aspect of the subject matter disclosed herein, a unique MR or MR LBD polypeptide is generated. Such a mutant can facilitate purification and the study of the structure and the ligand-binding abilities of an MR polypeptide, including the binding properties to a weak ligand. Thus, an aspect of the presently disclosed subject matter involves the use of both targeted and random mutagenesis of the MR gene for the production of a recombinant protein with improved solution characteristics for the purpose of crystallization, characterization of biologically relevant protein-protein interactions, compound screening assays, or for the production of a recombinant polypeptide having other characteristics of interest. Expression of the polypeptide in bacteria, preferably E. coli, is also an aspect of the presently disclosed subject matter.

In one embodiment, targeted mutagenesis was performed using a sequence alignment of several nuclear receptors, primarily steroid receptors. Several residues that differed in MR from other receptors were chosen for mutagenesis. Mutations were made to change these residues in an attempt to improve the solubility and stability of expressed MR LBD. Table 2 immediately below presents a list of mutations that were made and tested for expression in E. coli.

TABLE 2
Mutations of the MR LBD Gene for
Testing Solution Solubility and Stability

| Single mutations | Double mutations |
|---|---|
| C808S | C808S/S810L |
| S810L | |

Random mutagenesis can also be performed on residues where a significant difference (for example, hydrophobic versus hydrophilic) is observed between MR and other steroid receptors based on sequence alignment. Such positions can be randomized by oligo-directed or cassette mutagenesis. An MR LBD protein library can be sorted by an appropriate display system to select mutants with improved solution properties. Residues in MR that meet the criteria for such an approach include: C808S and S810L. In addition, residues predicted to neighbor these positions could also be randomized.

A method of modifying a test MR polypeptide is thus disclosed. The method can comprise: providing a test MR polypeptide sequence having a characteristic that is targeted for modification; aligning the test MR polypeptide sequence with at least one reference MR polypeptide sequence for which an X-ray structure is available, wherein the at least one reference MR polypeptide sequence has a characteristic that is desired for the test MR polypeptide; building a three-dimensional model for the test MR polypeptide using the three-dimensional coordinates of the X-ray structure(s) of the at least one reference polypeptide and its sequence alignment with the test MR polypeptide sequence; examining the three-dimensional model of the test MR polypeptide for differences with the at least one reference polypeptide that are associated with the desired characteristic; and mutating at least one amino acid residue in the test MR polypeptide sequence located at a difference identified above to a residue associated with the desired characteristic, whereby the test MR polypeptide is modified. By the term “associated with a desired characteristic” it is meant that a residue is found in the reference polypeptide at a point of difference wherein the difference provides a desired characteristic or phenotype in the reference polypeptide.

A method of modifying a test MR polypeptide to improve solubility and/or ligand binding properties in solution and the ability to form ordered crystals is also disclosed herein. In a preferred embodiment, the method comprises: (a) providing a test MR polypeptide sequence with unsatisfactory solubility, ligand binding or crystal forming properties; (b) mutating one or more amino acid residues in the test MR polypeptide to create a mutated polypeptide with improved solubility or crystal forming properties; (c) analyzing the mutated polypeptide for solubility and crystal forming properties; and (d) repeating the above steps a desired number of times until the mutated polypeptide has the desired solubility, ligand binding or crystal forming properties.

By the term “modifying” is meant any change in the solubility, ligand binding or crystal forming properties of the test MR polypeptide, including preferably a change to make the polypeptide more soluble. Such approaches to obtain soluble proteins for crystallization studies have been successfully demonstrated in the case of HIV integration integrase and the human leptin cytokine. See Dyda, F., et al., Science (1994) Dec. 23; 266(5193):1981-6; and Zhang et al., Nature (1997) May 8; 387(6629):206-9.

Typically, such a change can involve substituting a residue that is more hydrophilic than the wild type residue, however desirable changes are not limited to only hydrophobic/hydrophilic changes. Hydrophobicity and hydrophilicity criteria and comparison information are set forth herein below.

A method for modifying a test MR polypeptide to alter and preferably improve the solubility, stability in solution and other solution behavior, to alter and preferably improve the folding and stability of the folded structure, to improve the ability to form soluble complexes with weakly binding ligands, and to alter and preferably improve the ability to form ordered crystals is also provided herein. The aforementioned characteristics are representative “desired” or “predetermined” characteristics or phenotypes.

In some embodiments, the method comprises first providing a test MR polypeptide sequence for which the solubility, stability in solution, other solution behavior, tendency to fold properly, ability to form ordered crystals, or combination thereof is different from that desired and then measuring the affinity of mutated MR polypeptides for weakly binding ligands and selecting those mutations that give increased binding.

The method in some embodiments next comprises measuring the ability of mutated MR polypeptides to recruit co-factor peptides in the presence of partial agonist ligands and selecting those mutations that give increased binding, followed by aligning the test MR polypeptide sequence with the sequences of other reference NR polypeptides for which the X-ray structure is available and for which the solution properties, folding behavior and crystallization properties are closer to those desired.

The method then comprises in some embodiments building a three-dimensional model for the test MR polypeptide using the three-dimensional coordinates of the X-ray structure(s) of one or more of the reference polypeptides and their sequence alignment with the test MR polypeptide sequence. Optionally, the method can then comprise optimizing the side-chain conformations in the three-dimensional model by generating many alternative side-chain conformations, refining by energy minimization, and selecting side-chain conformations with lower energy. The three-dimensional model is then examined for the test MR graphically for lipophilic side-chains that are exposed to solvent, for clusters of two or more lipophilic side-chains exposed to solvent, for lipophilic pockets and clefts on the surface of the protein model, and in particular for sites on the surface of the protein model that are more lipophilic than the corresponding sites on the structure(s) of the reference NR polypeptide.

For each residue identified in the immediately proceeding step, the method then comprises in some embodiments mutating the amino acid to an amino acid with different hydrophilicity, and usually to a more hydrophilic amino acid, whereby the exposed lipophilic sites are reduced, and the solution properties improved. The three-dimensional model is then examined graphically at each site where the amino acid in the test MR polypeptide is different from the amino acid at the corresponding position in the reference NR polypeptide, and checked as to whether the amino acid in the test MR polypeptide makes favorable interactions with the atoms that lie around it in the three-dimensional model, considering the side-chain conformations predicted in the above steps, as well as likely alternative conformations of the side-chains, and also considering the possible presence of water molecules (for this analysis, an amino acid is considered to make “favorable interactions with the atoms that lie around it” if these interactions are more favorable than the interactions that would be obtained if it was replaced by any of the 19 other naturally-occurring amino acids).

For each residue identified above as not making favorable interactions with the atoms that lie around it, the residue can be mutated to another amino acid that can make better interactions with the atoms that lie around it, thereby promoting the tendency for the test MR polypeptide to fold into a stable structure with improved solution properties, less tendency to unfold, and greater tendency to form ordered crystals.

In some embodiments, the three-dimensional model is examined graphically at each residue position where the amino acid in the test MR polypeptide is different from the amino acid at the corresponding position in the reference NR polypeptide, and checking whether the steric packing, hydrogen bonding and other energetic interactions could be improved by mutating that residue or any one or more of the surrounding residues lying within 8 angstroms in the three-dimensional model. For each residue position identified as potentially allowing an improvement in the packing, hydrogen bonding and energetic interactions, mutating those residues individually or in combination to residues that can improve the packing, hydrogen bonding and energetic interactions is performed, thereby promoting the tendency for the test MR polypeptide to fold into a stable structure with improved solution properties, less tendency to unfold, and greater tendency to form ordered crystals.

By the term “graphically” it is meant through the use of computer aided graphics, such as by the use of a software package disclosed herein. Optionally, in this embodiment, the reference NR polypeptide is MR.

An isolated MR polypeptide, or functional portion thereof, comprising one or more mutations in an LBD, wherein the mutation alters the solubility, ligand binding or crystallization properties of the LBD, is also disclosed. Preferably, in each case, the mutation can be at a residue selected from the group consisting of C808 and S810 and combinations thereof. More preferably, the mutation is selected from the group consisting of C808L and S810L and combinations thereof. Even more preferably, the mutation is made by targeted point or randomizing mutagenesis. Hydrophobicity and hydrophilicity, and other criteria and comparison information are set forth herein below.

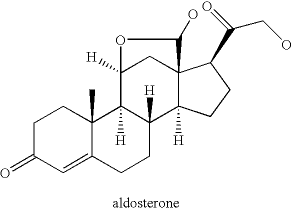

As used herein, the terms “engineered MR”, “engineered MR LDB”, “NR, SR or MR mutant”, and “MR LBD mutant” refers to polypeptides having amino acid sequences that contain at least one mutation in the wild-type sequence, including at an analogous position in any polypeptide based on a sequence alignment to MR. The terms also refer to MR and MR LBD polypeptides which are capable of exerting a biological effect in that they comprise all or a part of the amino acid sequence of an engineered mutant polypeptide of the presently disclosed subject matter, or cross-react with antibodies raised against an engineered mutant polypeptide, or retain all or some or an enhanced degree of the biological activity of the engineered mutant amino acid sequence or protein. Such biological activity can include the binding of small molecules in general, the binding of mineralocorticoids in particular and even more particularly the binding of aldosterone.

The terms “engineered MR LBD” and “MR LBD mutant” also includes analogs of an engineered MR polypeptide or MR LBD or MR LBD mutant polypeptide. By “analog” is intended that a DNA or polypeptide sequence can contain alterations relative to the sequences disclosed herein, yet retain all or some or an enhanced degree of the biological activity of those sequences. Analogs can be derived from genomic nucleotide sequences or from other organisms, or can be created synthetically. Those of skill in the art will appreciate that other analogs, as yet undisclosed or undiscovered, can be used to design and/or construct mutant analogs. There is no need for an engineered mutant polypeptide to comprise all or substantially all of the amino acid sequence of the wild type polypeptide (e.g. SEQ ID NO:4). Shorter or longer sequences are anticipated to be of use with the subject matter disclosed herein; shorter sequences are herein referred to as “segments”. Thus, the terms “engineered MR LBD” and “MR LBD mutant” also includes fusion, chimeric or recombinant engineered MR LBD or MR LBD mutant polypeptides and proteins comprising sequences of the present subject matter. Methods of preparing such proteins are disclosed herein above.

VII.C. Sequence Similarity and Identity

As used herein, the term “substantially similar” as applied to MR means that a particular sequence varies from nucleic acid sequence of any of odd numbered SEQ ID NOs:1-9, or the amino acid sequence of any of even numbered SEQ ID NOs:2-10 by one or more deletions, substitutions, or additions, the net effect of which is to retain at least some of the biological activity of the natural gene, gene product, or sequence. Such sequences include “mutant” or “polymorphic” sequences, or sequences in which the biological activity and/or the physical properties are altered to some degree, but retain at least some or an enhanced degree of the original biological activity and/or physical properties. In determining nucleic acid sequences, all subject nucleic acid sequences capable of encoding substantially similar amino acid sequences are considered to be substantially similar to a reference nucleic acid sequence, regardless of differences in codon sequences or substitution of equivalent amino acids to create biologically functional equivalents.

VII.C.1. Sequences Substantially Identical to an Engineered MR, or MR LBD Mutant Sequence Nucleic acids that are substantially identical to a nucleic acid sequence of an engineered MR or MR LBD mutant of the presently disclosed subject matter, e.g. allelic variants, genetically altered versions of the gene, etc., bind to an engineered MR or MR LBD mutant sequence under stringent hybridization conditions. By using probes, particularly labeled probes of DNA sequences, one can isolate homologous or related genes. The source of homologous genes can be any species, e.g. primate species; rodents, such as rats and mice, canines, felines, bovines, equines, yeast, nematodes, etc.

Between mammalian species, e.g. human and mouse, homologs have substantial sequence similarity, i.e. at least 75% sequence identity between nucleotide sequences. Sequence similarity is calculated based on a reference sequence, which can be a subset of a larger sequence, such as a conserved motif, coding region, flanking region, etc. A reference sequence will usually be at least about 18 nt long, more usually at least about 30 nt long, and can extend to the complete sequence that is being compared. Algorithms for sequence analysis are known in the art, such as BLAST, described in Altschul et al., (1990) J. Mol. Biol. 215: 403-10. Software for performing BLAST analyses is publicly available through the National Center for Biotechnology Information (http://www.ncbi.nlm.nih.gov/).

This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength W=11, an expectation E=10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See Henikoff & Henikoff, (1989) Proc Natl Acad Sci U.S.A. 89:10915.

In addition to calculating percent sequence identity, the BLAST algorithm also performs a statistical analysis of the similarity between two sequences. See, e.g., Karlin and Altschul, (1993) Proc Natl Acad Sci U.S.A. 90: 5873-5887. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

Percent identity or percent similarity of a DNA or peptide sequence can be determined, for example, by comparing sequence information using the GAP computer program, available from the University of Wisconsin Geneticist Computer Group. The GAP program utilizes the alignment method of Needleman et al., (1970) J. Mol. Biol. 48: 443, as revised by Smith et al., (1981) Adv. Appl. Math. 2:482. Briefly, the GAP program defines similarity as the number of aligned symbols (i.e., nucleotides or amino acids), which are similar, divided by the total number of symbols in the shorter of the two sequences. The preferred parameters for the GAP program are the default parameters, which do not impose a penalty for end gaps. See, e.g., Schwartz et al., eds., (1979), Atlas of Protein Sequence and Structure, National Biomedical Research Foundation, pp. 357-358, and Gribskov et al., (1986) Nucl. Acids. Res. 14: 6745.

The term “similarity” is contrasted with the term “identity”. Similarity is defined as above; “identity”, however, means a nucleic acid or amino acid sequence having the same amino acid at the same relative position in a given family member of a gene family. Homology and similarity are generally viewed as broader terms than the term identity. Biochemically similar amino acids, for example leucine/isoleucine or glutamate/aspartate, can be present at the same position—these are not identical per se, but are biochemically “similar.” As disclosed herein, these are referred to as conservative differences or conservative substitutions. This differs from a conservative mutation at the DNA level, which changes the nucleotide sequence without making a change in the encoded amino acid, e.g. TCC to TCA, both of which encode serine.

As used herein, DNA analog sequences are “substantially identical” to specific DNA sequences disclosed herein if: (a) the DNA analog sequence is derived from coding regions of the nucleic acid sequence shown in any one of odd numbered SEQ ID NOs:1-9 or (b) the DNA analog sequence is capable of hybridization with DNA sequences of (a) under stringent conditions and which encode a biologically active MR or MR LBD gene product; or (c) the DNA sequences are degenerate as a result of alternative genetic code to the DNA analog sequences defined in (a) and/or (b). Substantially identical analog proteins and nucleic acids will have between about 70% and 80%, preferably between about 81% to about 90% or even more preferably between about 91% and 99% sequence identity with the corresponding sequence of the native protein or nucleic acid. Sequences having lesser degrees of identity but comparable biological activity are considered to be equivalents.

As used herein, “stringent conditions” means conditions of high stringency, for example 6× SSC, 0.2% polyvinylpyrrolidone, 0.2% Ficoll, 0.2% bovine serum albumin, 0.1% sodium dodecyl sulfate, 100 jg/ml salmon sperm DNA and 15% formamide at 68° C. For the purposes of specifying additional conditions of high stringency, preferred conditions are salt concentration of about 200 mM and temperature of about 45° C. One example of such stringent conditions is hybridization at 4× SSC, at 65° C., followed by a washing in 0.1×SSC at 65° C. for one hour. Another exemplary stringent hybridization scheme uses 50% formamide, 4× SSC at 42° C.

In contrast, nucleic acids having sequence similarity are detected by hybridization under lower stringency conditions. Thus, sequence identity can be determined by hybridization under lower stringency conditions, for example, at 50° C. or higher and 0.1× SSC (9 mM NaCl/0.9 mM sodium citrate) and the sequences will remain bound when subjected to washing at 55° C. in 1× SSC.

As used herein, the term “complementary sequences” means nucleic acid sequences that are base-paired according to the standard Watson-Crick complementarity rules. The present disclosure also encompasses the use of nucleotide segments that are complementary to the sequences disclosed herein.

Hybridization can also be used for assessing complementary sequences and/or isolating complementary nucleotide sequences. As discussed above, nucleic acid hybridization will be affected by such conditions as salt concentration, temperature, or organic solvents, in addition to the base composition, length of the complementary strands, and the number of nucleotide base mismatches between the hybridizing nucleic acids, as will be readily appreciated by those skilled in the art. Stringent temperature conditions will generally include temperatures in excess of about 30° C., typically in excess of about 37° C., and preferably in excess of about 45° C. Stringent salt conditions will ordinarily be less than about 1,000 mM, typically less than about 500 mM, and preferably less than about 200 mM. However, the combination of parameters is much more important than the measure of any single parameter. See, e.g., Wetmur & Davidson, (1968) J. Mol. Biol. 31: 349-70. Determining appropriate hybridization conditions to identify and/or isolate sequences containing high levels of homology is well known in the art. See, e.g., Sambrook et al., (1989) Molecular Cloning: A Laboratory Manual, Cold Spring Harbor, N.Y.

VII.C.2. Functional Equivalents of an Engineered MR or MR LBD Mutant Nucleic Acid Sequence

As used herein, the term “functionally equivalent codon” is used to refer to codons that encode the same amino acid, such as the ACG and AGU codons for serine. For example, MR or MR LBD-encoding nucleic acid sequences comprising any one of odd numbered SEQ ID NOs:1-9, which have functionally equivalent codons are covered by the subject matter disclosed herein. Thus, when referring to the sequence example presented in odd numbered SEQ ID NOs:1-9, applicants provide substitution of functionally equivalent codons into the sequence example of in odd numbered SEQ ID NOs:1-9. Thus, applicants are in possession of amino acid and nucleic acids sequences which include such substitutions but which are not set forth herein in their entirety for convenience.

It will also be understood by those of skill in the art that amino acid and nucleic acid sequences can include additional residues, such as additional N- or C-terminal amino acids or 5′ or 3′ nucleic acid sequences, and yet still be essentially as set forth in one of the sequences disclosed herein, so long as the sequence retains biological protein activity where polypeptide expression is concerned. The addition of terminal sequences particularly applies to nucleic acid sequences which can, for example, include various non-coding sequences flanking either of the 5′ or 3′ portions of the coding region or can include various internal sequences, i.e., introns, which are known to occur within genes.

VII.C.3. Biological Equivalents

The present subject matter envisions and includes biological equivalents of an engineered MR or MR LBD mutant polypeptide of the presently disclosed subject matter. The term “biological equivalent” refers to proteins having amino acid sequences which are substantially identical to the amino acid sequence of an engineered MR LBD mutant of the present subject matter and which are capable of exerting a biological effect in that they are capable of binding small molecules or cross-reacting with anti-MR or MR LBD mutant antibodies raised against an engineered mutant MR or MR LBD polypeptide of the presently disclosed subject matter.

For example, certain amino acids can be substituted for other amino acids in a protein structure without appreciable loss of interactive capacity with, for example, structures in the nucleus of a cell. Since it is the interactive capacity and nature of a protein that defines that protein's biological functional activity, certain amino acid sequence substitutions can be made in a protein sequence (or the nucleic acid sequence encoding it) to obtain a protein with the same, enhanced, or antagonistic properties. Such properties can be achieved by interaction with the normal targets of the protein, but this need not be the case, and the biological activity of the disclosed herein is not limited to a particular mechanism of action. It is thus in accordance with the subject matter disclosed herein that various changes can be made in the amino acid sequence of an engineered MR or MR LBD mutant polypeptide of the present subject matter or its underlying nucleic acid sequence without appreciable loss of biological utility or activity.

Biologically equivalent polypeptides, as used herein, are polypeptides in which certain, but not most or all, of the amino acids can be substituted. Thus, when referring to the sequence examples presented in any of even numbered SEQ ID NOs:2-10, applicants envision substitution of codons that encode biologically equivalent amino acids, as described herein, into a sequence example of even numbered SEQ ID NOs: 2-10, respectively. Thus, applicants are in possession of amino acid and nucleic acids sequences which include such substitutions but which are not set forth herein in their entirety for convenience.

Alternatively, functionally equivalent proteins or peptides can be created via the application of recombinant DNA technology, in which changes in the protein structure can be engineered, based on considerations of the properties of the amino acids being exchanged, e.g. substitution of lie for Leu. Changes designed by man can be introduced through the application of site-directed mutagenesis techniques, e.g., to introduce improvements to the antigenicity of the protein or to test an engineered mutant polypeptide of the present subject matter in order to modulate ligand binding or other activity, at the molecular level.

Amino acid substitutions, such as those which might be employed in modifying an engineered mutant polypeptide disclosed herein are generally, but not necessarily, based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all of similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents. Those of skill in the art will appreciate other biologically functionally equivalent changes. It is implicit in the above discussion, however, that one of skill in the art can appreciate that a radical, rather than a conservative substitution is warranted in a given situation. Non-conservative substitutions in engineered mutant LBD polypeptides are also an aspect of the presently disclosed subject matter.

In making biologically functional equivalent amino acid substitutions, the hydropathic index of amino acids can be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kvte & Doolittle, (1982), J. Mol. Biol. 157: 105-132, incorporated herein by reference). It is known that certain amino acids can be substituted for other amino acids having a similar hydropathic index or score and still retain a similar biological activity. In making changes based upon the hydropathic index, the substitution of amino acids whose hydropathic indices are within ±2 of the original value is preferred, those that are within ±1 of the original value are particularly preferred, and those within ±0.5 of the original value are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with its immunogenicity and antigenicity, i.e. with a biological property of the protein. It is understood that an amino acid can be substituted for another having a similar hydrophilicity value and still obtain a biologically equivalent protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 of the original value is preferred, those that are within ±1 of the original value are particularly preferred, and those within ±0.5 of the original value are even more particularly preferred.

While discussion has focused on functionally equivalent polypeptides arising from amino acid changes, it will be appreciated that these changes can be effected by alteration of the encoding DNA, taking into consideration also that the genetic code is degenerate and that two or more codons can code for the same amino acid.

Thus, it will also be understood that the subject matter disclosed herein is not limited to the particular amino acid and nucleic acid sequences of any of SEQ ID NOs:1-10. Recombinant vectors and isolated DNA segments can therefore variously include an engineered MR or MR LBD mutant polypeptide-encoding region itself, include coding regions bearing selected alterations or modifications in the basic coding region, or include larger polypeptides which nevertheless comprise an MR or MR LBD mutant polypeptide-encoding regions or can encode biologically functional equivalent proteins or polypeptides which have variant amino acid sequences. Biological activity of an engineered MR or MR LBD mutant polypeptide can be determined, for example, by transcription assays known to those of skill in the art.

The nucleic acid segments disclosed herein, regardless of the length of the coding sequence itself, can be combined with other DNA sequences, such as promoters, enhancers, polyadenylation signals, additional restriction enzyme sites, multiple cloning sites, other coding segments, and the like, such that their overall length can vary considerably. It is therefore contemplated that a nucleic acid fragment of almost any length can be employed, with the total length preferably being limited by the ease of preparation and use in the intended recombinant DNA protocol. For example, nucleic acid fragments can be prepared which include a short stretch complementary to a nucleic acid sequence set forth in any of odd numbered SEQ ID NOs:1-9, such as about 10 nucleotides, and which are up to 10,000 or 5,000 base pairs in length. DNA segments with total lengths of about 4,000, 3,000, 2,000, 1,000, 500, 200, 100, and about 50 base pairs in length are also useful.

The DNA segments disclosed herein encompass biologically functional equivalents of engineered MR or MR LBD mutant polypeptides. Such sequences can rise as a consequence of codon redundancy and functional equivalency that are known to occur naturally within nucleic acid sequences and the proteins thus encoded. Alternatively, functionally equivalent proteins or polypeptides can be created via the application of recombinant DNA technology, in which changes in the protein structure can be engineered, based on considerations of the properties of the amino acids being exchanged. Changes can be introduced through the application of site-directed mutagenesis techniques, e.g., to introduce improvements to the antigenicity of the protein or to test variants of an engineered mutant disclosed herein in order to examine the degree of binding activity, or other activity at the molecular level. Various site-directed mutagenesis techniques are known to those of skill in the art and can be employed with the subject matter disclosed herein.

The presently disclosed subject matter further encompasses fusion proteins and peptides wherein an engineered mutant MR coding region is aligned within the same expression unit with other proteins or peptides having desired functions, such as for purification or immunodetection purposes.

Recombinant vectors form important further aspects of the subject matter disclosed herein. Particularly useful vectors are those in which the coding portion of the DNA segment is positioned under the control of a promoter. The promoter can be that naturally associated with an MR gene, as can be obtained by isolating the 5′ non-coding sequences located upstream of the coding segment or exon, for example, using recombinant cloning and/or PCR technology and/or other methods known in the art, in conjunction with the compositions disclosed herein.

In other embodiments, certain advantages will be gained by positioning the coding DNA segment under the control of a recombinant, or heterologous, promoter. As used herein, a recombinant or heterologous promoter is a promoter that is not normally associated with an MR gene in its natural environment. Such promoters can include promoters isolated from bacterial, viral, eukaryotic, or mammalian cells. Naturally, it will be important to employ a promoter that effectively directs the expression of the DNA segment in the cell type chosen for expression. The use of promoter and cell type combinations for protein expression is generally known to those of skill in the art of molecular biology (See, e.g., Sambrook et al., (1989) Molecular Cloning: A Laboratory Manual, Cold Spring Harbor Laboratory, New York, specifically incorporated herein by reference). The promoters employed can be constitutive or inducible and can be used under the appropriate conditions to direct high level expression of the introduced DNA segment, such as is advantageous in the large-scale production of recombinant proteins or peptides. One preferred promoter system contemplated for use in high-level expression is a T7 promoter-based system.

VII.D. Antibodies to an Engineered MR or MR LBD Mutant Polvpeptide

The presently disclosed subject matter also provides an antibody that specifically binds an engineered MR or MR LBD mutant polypeptide and methods to generate same. The term “antibody” indicates an immunoglobulin protein, or functional portion thereof, including a polyclonal antibody, a monoclonal antibody, a chimeric antibody, a single chain antibody, Fab fragments, and a Fab expression library. “Functional portion” refers to the part of the protein that binds a molecule of interest. In a preferred embodiment, an antibody is a monoclonal antibody. Techniques for preparing and characterizing antibodies are well known in the art (See, e.g., Harlow & Lane (1988) Antibodies: A Laboratorv Manual, Cold Spring Harbor Laboratory Press, Cold Spring Harbor, N.Y.). A monoclonal antibody of the present subject matter can be readily prepared through use of well-known techniques such as the hybridoma techniques exemplified in U.S. Patent No 4,196,265 and the phage-displayed techniques disclosed in U.S. Pat. No. 5,260,203.

The phrase “specifically (or selectively) binds to an antibody”, or “specifically (or selectively) immunoreactive with”, when referring to a protein or peptide, refers to a binding reaction which is determinative of the presence of the protein in a heterogeneous population of proteins and other biological materials. Thus, under designated immunoassay conditions, the specified antibodies bind to a particular protein and do not show significant binding to other proteins present in the sample. Specific binding to an antibody under such conditions can require an antibody that is selected for its specificity for a particular protein. For example, antibodies raised to a protein with an amino acid sequence encoded by any of the nucleic acid sequences of the present subject matter can be selected to obtain antibodies specifically immunoreactive with that protein and not with unrelated proteins.

The use of a molecular cloning approach to generate antibodies, particularly monoclonal antibodies, and more particularly single chain monoclonal antibodies, are also provided. The production of single chain antibodies has been described in the art. See, e.g., U.S. Pat. No. 5,260,203. For this approach, combinatorial immunoglobulin phagemid libraries are prepared from RNA isolated from the spleen of the immunized animal, and phagemids expressing appropriate antibodies are selected by panning on endothelial tissue. The advantages of this approach over conventional hybridoma techniques are that approximately 10⁴ times as many antibodies can be produced and screened in a single round, and that new specificities are generated by heavy (H) and light (L) chain combinations in a single chain, which further increases the chance of finding appropriate antibodies. Thus, an antibody disclosed herein, or a “derivative” of an antibody thereof, pertains to a single polypeptide chain binding molecule which has binding specificity and affinity substantially similar to the binding specificity and affinity of the light and heavy chain aggregate variable region of an antibody described herein.

The term “immunochemical reaction”, as used herein, refers to any of a variety of immunoassay formats used to detect antibodies specifically bound to a particular protein, including but not limited to competitive and non-competitive assay systems using techniques such as radioimmunoassays, ELISA (enzyme linked immunosorbent assay), “sandwich” immunoassays, immunoradiometric assays, gel diffusion precipitation reactions, immunodiffusion assays, in situ immunoassays (e.g., using colloidal gold, enzyme or radioisotope labels), western blots, precipitation reactions, agglutination assays (e.g., gel agglutination assays, hemagglutination assays), complement fixation assays, immunofluorescence assays, protein A assays, and immunoelectrophoresis assays, etc. See Harlow & Lane (1988) for a description of immunoassay formats and conditions.

VII.E. Method for Detecting an Engineered MR or MR LBD Mutant Polypeptide or an Nucleic Acid Molecule Encoding the Same

In another aspect of the subject matter disclosed herein, a method is provided for detecting a level of an engineered MR or MR LBD mutant polypeptide using an antibody that specifically recognizes an engineered MR or MR LBD mutant polypeptide, or portion thereof. In a preferred embodiment, biological samples from an experimental subject and a control subject are obtained, and an engineered MR or MR LBD mutant polypeptide is detected in each sample by immunochemical reaction with the antibody. More preferably, the antibody recognizes amino acids of any one of the even-numbered SEQ ID NOs:2-10, and is prepared according to a method as disclosed herein for producing such an antibody.

In one embodiment, an antibody is used to screen a biological sample for the presence of an engineered MR or MR LBD mutant polypeptide. A biological sample to be screened can be a biological fluid such as extracellular or intracellular fluid, or a cell or tissue extract or homogenate. A biological sample can also be an isolated cell (e.g., in culture) or a collection of cells such as in a tissue sample or histology sample. A tissue sample can be suspended in a liquid medium or fixed onto a solid support such as a microscope slide. In accordance with a screening assay method, a biological sample is exposed to an antibody immunoreactive with an engineered MR or MR LBD mutant polypeptide whose presence is being assayed, and the formation of antibody-polypeptide complexes is detected. Techniques for detecting such antibody-antigen conjugates or complexes are well known in the art and include but are not limited to centrifugation, affinity chromatography and the like, and binding of a labeled secondary antibody to the antibody-candidate receptor complex.

In another aspect of the presently disclosed subject matter, a method is provided for detecting a nucleic acid molecule that encodes an engineered MR or MR LBD mutant polypeptide. According to the method, a biological sample having nucleic acid material is procured and hybridized under stringent hybridization conditions to an engineered MR or MR LBD mutant polypeptide-encoding nucleic acid molecule disclosed herein. Such hybridization enables a nucleic acid molecule of the biological sample and an engineered MR or MR LBD mutant polypeptide encoding-nucleic acid molecule to form a detectable duplex structure. Preferably, the engineered MR or MR LBD mutant polypeptide encoding-nucleic acid molecule includes some or all nucleotides of any one of the odd-numbered SEQ ID NOs:1-9. Also preferably, the biological sample comprises human nucleic acid material.

VIII. Formation of MR Ligand Binding Domain Crvstals

In one embodiment, provided herein are crystals of Native MR LBD. The crystals were obtained using the methodology disclosed in the Examples. The MR LBD crystals, which can be native crystals, derivative crystals or co-crystals, have orthorhombic unit cells, wherein α=0=γ=90° and having space group symmetry C222, with unit cell dimensions of α=93.0 Å, b=173.6 Å, c=42.4 Å. In this crystal form, there is 1 MR LBD molecule in the asymmetric unit. This crystal form can be formed in a crystallization reservoir as described in the Examples.

The crystals of native MR complexed with deoxycorticosterone diffracted to 2.36 Å at the APS/IMCA beam line 17BM. The final model was refined to R=24.0%, R_{free}=27.3%, and contained MR residues 727-908, and 914-984, one molecule of deoxycorticosterone, and 43 water molecules. As seen in other steroid receptor LBDs, the extension of MR C-terminal to the AF2 interacts with helix 10 via hydrogen bonds between D929 and the amide nitrogens of F981, and H982. The most unusual feature of the structure is that there is clear protein density N-terminal to helix 1 (residues 727-737). The protein was ordered, and residues formed a short helix that bound near the co-activator groove of a crystallographically related molecule. This N-terminal feature was present in all MR complexes with steroid ligands, and had the unintended consequence of stabilizing the LBD in the active conformation.

In another embodiment, provided herein, are crystals of MR LBD as a complex with a peptide from a nuclear receptor co-activator. These crystals were obtained using the methodology disclosed in the Examples. The MR LBD crystals, which can be native crystals, derivative crystals or co-crystals, have monoclinic unit cells, wherein α=γ=90°, β=94.24° and having space group symmetry P2₁, and the unit cell has dimensions of a=40.1 Å, b=80.6 Å, and c=116.9 Å. In the example of this crystal form specified, there are two MR LBD molecules in the asymmetric unit, one molecule of a peptide containing amino acids 732-756 of the nuclear receptor co-activator TIF2 per MR LBD, and one molecule of the ligand (deoxycorticosterone) per MR LBD. This crystal form can be formed in a crystallization reservoir as described in the Examples.

VIII.A. Preparation of MR Crvstals

The native and derivative co-crystals, and fragments thereof, disclosed herein can be obtained by a variety of techniques, including batch, liquid bridge, dialysis, vapor diffusion and hanging drop methods (See, e.g., McPherson, (1982) Preparation and Analysis of Protein Crystals, John Wiley, New York; McPherson, (1990) Eur. J. Biochem. 189:1-23; Weber, (1991) Adv. Protein Chem. 41:1-36). In a preferred embodiment, the vapor diffusion and hanging drop methods are used for the crystallization of MR polypeptides and fragments thereof. A more preferred hanging drop method technique is disclosed in the Examples.

In general, native crystals disclosed herein are grown by dissolving substantially pure MR polypeptide or a fragment thereof in an aqueous buffer containing a precipitant at a concentration just below that necessary to precipitate the protein. Water is removed by controlled evaporation to produce precipitating conditions, which are maintained until crystal growth ceases.

In one embodiment, native crystals are grown by vapor diffusion (See. eg., McPherson, (1982)Preparation and Analysis of Protein Crystals, John Wiley, New York.; McPherson, (1990) Eur. J. Biochem. 189:1-23). In this method, the polypeptide/precipitant solution is allowed to equilibrate in a closed container with a larger aqueous reservoir having a precipitant concentration optimal for producing crystals. Generally, less than about 25 μL of MR polypeptide solution is mixed with an equal volume of reservoir solution, giving a precipitant concentration about half that required for crystallization. This solution is suspended as a droplet underneath a coverslip, which is sealed onto the top of the reservoir. The sealed container is allowed to stand, until crystals grow. Crystals generally form within two to six weeks, and are suitable for data collection within approximately seven to ten weeks. Of course, those of skill in the art will recognize that the above-described crystallization procedures and conditions can be varied.

VIII.B. Preparation of Derivative Crystals

Derivative crystals of the subject matter disclosed herein, e.g. heavy atom derivative crystals, can be obtained by soaking native crystals in mother liquor containing salts of heavy metal atoms. Such derivative crystals are useful for phase analysis in the solution of crystals disclosed herein. In a preferred embodiment, for example, soaking a native crystal in a solution containing methyl-mercury chloride provides derivative crystals suitable for use as isomorphous replacements in determining the X-ray crystal structure of an MR polypeptide. Additional reagents useful for the preparation of the derivative crystals of the present subject matter will be apparent to those of skill in the art after review of the presently disclosed subject matter.

VIII.C. Preparation of Co-crystals

Co-crystals of the presently disclosed subject matter can be obtained by soaking a native crystal in mother liquor containing compounds known or predicted to bind the LBD of an MR, or a fragment thereof. Alternatively, co-crystals can be obtained by co-crystallizing an MR LBD polypeptide or a fragment thereof in the presence of one or more compounds known or predicted to bind the polypeptide, for example aldosterone. Using mutated forms of an MR LBD, as described herein, a co-crystal of the mutant MR LBD and a weakly binding ligand can be formed. This allows the mutant MR LBD to serve as a surrogate for the native receptors of these ligands and allows for the determination of crystal structures of the mutant MR bound to the other receptor's native ligands and novel, weakly binding non-steroidal ligands, which cannot be obtained with the AR, PR and GR constructs currently in existence. Alternatively, co-crystals of MR with various ligands can be prepared using cross-seeding techniques wherein microscopic fragments of existing native or mutant MR crystals are used as nucleation centers to grow MR crystals with other ligands. In some embodiments, as disclosed in the Examples, such compounds include, for example, deoxycorticosterone and progesterone.

VIII.D. Solving a Crvstal Structure

Crystal structures disclosed herein can be solved using a variety of techniques including, but not limited to, isomorphous replacement, anomalous scattering or molecular replacement methods. Computer software packages are also helpful in solving a crystal structure disclosed herein. Applicable software packages include but are not limited to the CCP4 package disclosed in the Examples, the X-PLORT program (Brunner, (1992) X-PLOR, Version 3.1. A System for X-ray Crystallography and NMR, Yale University Press, New Haven, Conn.; X-PLOR is available from Molecular Simulations, Inc., San Diego, Calif.), Xtal View (McRee, (1992) J. Mol. Graphics 10: 44-46; X-tal View is available from the San Diego Supercomputer Center). SHELXS 97 (Sheldrick (1990) Acta Cryst. A46: 467; SHELX 97 is available from the Institute of Inorganic Chemistry, Georg-August-Universitatt, Gottingen, Germany), HEAVY (Terwilliger, Los Alamos National Laboratory) and SHAKE-AND-BAKE (Hauptman, (1997) Curr. Opin. Struct Biol. 7: 672-80; Weeks et al., (1993) Acta Cryst. D49: 179; available from the Hauptman-Woodward Medical Research Institute, Buffalo, N.Y.) can be used. See also, Ducruix & Geige, (1992) Crystallization of Nucleic Acids and Proteins: A Practical Approach, IRL Press, Oxford, England, and references cited therein.

IX. Characterization and Solution of an MR Ligand Binding Domain Crvstal

Referring now to FIGS. 1A and 1B (rotated 90° from 1A view), the overall arrangement of the MR LBD is depicted in a ribbon diagram that was derived from the crystalline polypeptide disclosed herein. The MR/deoxycorticosterone crystal structure data is shown in Table 4 in the Examples below. The MR LBD is shown in black ribbon representation with residues contacting the ligand colored gray. Overall, MR LBD has the same three-layered alpha-helical fold observed in other NR LBDs, with deoxycorticosterone bound in a fully enclosed pocket contacting residues in helices 3, 4, 5, 6, 7, and 11, and the β turn. The ligand is shown as a gray ball-and-stick structure bound in a fully enclosed pocket contacting residues in helices 3, 4, 5, 6, 7, and 11 and the β-turn.

Referring now to FIGS. 2A and 2B, the overlap of MR LBD with the LBDs of the AR, GR and PR is depicted in a ribbon diagram. The MR is shown in black, the AR is shown in dark gray, the GR is shown in gray and the PR is shown in light gray. Backbone C-alpha atoms are also shown. This superposition is consistent with the sequence alignment approach taken in the design of the MR LBD polypeptide disclosed herein. Obtaining the structure of the MR LBD as a complex with deoxycorticosterone permitted a direct comparison with the AR, GR, and PR receptors. Both sequentially and structurally, these proteins are most closely related to MR in the ligand-binding domain. The average root mean squared deviation between Cα atoms of the structurally conserved regions of MR (residues 763-972) and the corresponding residues in AR, GR, and PR, were 1.04 Å, 1.66 Å, and 1.11 Å, respectively. From both visual inspection of the aligned structures (FIG. 2A) and from the difference-distance plot (FIG. 2B) it appears that AR is closest to MR structurally, with PR and GR being closer to each other. The other steroid receptors deviate most from MR at residues 820-825 (βturn), 829-852 (helix 6-7), 906-915 (helix 9) and 951-972 (AF2 helix). Residues in steroid receptors corresponding to MR 829-852 are both close to the ligand and show significant deviations from MR. Docking studies based on homology models are expected to be more problematic for ligands that extend into this region of the protein.

It is also noted that, within the LBDs, the sequence identity is as follows:

TABLE 3
Sequence Identity of NR LBDs

| | MR | GR | PR | AR |
|---|---|---|---|---|
| MR | 100% | 56% | 55% | 51% |
| GR | 56% | 100% | 54% | 50% |
| PR | 55% | 54% | 100% | 55% |
| AR | 51% | 50% | 55% | 100% |

IX.A. Unique Structural Differences Between MR and Other SRs

Even though the MR LBD shares over 50% sequence identity with GR, PR and AR and fold into a similar three-layer helical sandwich (FIG. 2A), there are a number of unique structural differences in their structures. The other steroid receptors deviate most from MR at residues 820-825 (β-turn), 829-852 (helix 6-7), 906-915 (helix 9) and 951-972 (AF2 helix). Residues in steroid receptors corresponding to MR 829-852 are both close to the ligand and show significant deviations from MR. Docking studies based on homology models are expected to be more problematic for ligands that extend into this region of the protein.

These differences contribute a unique shape of the binding pocket for each receptor (FIG. 3) and might thus provide a molecular basis for steroid specificity of these receptors. The detailed structural information about the MR LBD and the pocket provided herein can be further exploited to design receptor specific agonists or antagonists.

Further, even with these differences, a mutant MR LBD capable of binding weak ligands can be used as a surrogate for studying the interactions of other NR receptors with their native as well as novel, weakly binding non-steroidal ligands using x-ray crystallography even where it is not currently possible to grow crystals of the NR receptors bound with these ligands. In one embodiment, a method of modeling the molecular interactions of an NR with its natural ligand. The method comprises first crystallizing an MR ligand binding domain polypeptide in complex with a ligand, wherein the MR ligand binding domain polypeptide comprises at least one mutation, and wherein the mutation improves ligand binding or crystal forming properties. Next, the MR ligand binding domain polypeptide is analyzed to determine the three-dimensional structure of the crystallized MR ligand binding domain polypeptide in complex with the ligand, whereby the three-dimensional structure of the crystallized MR ligand binding domain polypeptide in complex with the ligand acts as a surrogate for the NR in complex with its ligand and thereby models the molecular interactions of the NR with the ligand. In a some embodiments, a mutated MR can act as a surrogate for modeling molecular interactions of AR, PR or GR with novel, weakly binding non-steroidal ligands.

IX.B. Deoxycorticosterone

The ligand binding domain of MR was co-crystallized with deoxycorticosterone, which has the IUPAC name 21-hydroxypregn-4-ene-3,20-dione. It has a molecular weight of 330.5. The empirical formula for deoxycorticosterone is C₂₁H₃₀O₃.

The cortex of the adrenal gland secretes deoxycorticosterone. It has about three percent of the sodium-retaining activity of aldosterone. The acetate and pivalate salts are used for mineralocorticoid replacement therapy.

IX.C. Characterization of the MR LBD and Interactions Between MR and Deoxycorticosterone

Referring now to FIG. 3, the MR LBD pocket is depicted schematically. The MR LBD pocket is shown in a gray stick representation with key amino acid side chains shown as stick models. Deoxycorticosterone is shown as a ball-and-stick model interacting with the MR LBD at key residues. Water molecules are shown as dark gray spheres and hydrogen bonds between molecules are shown as dashed lines.

MR makes many of the same interactions with deoxycorticosterone that other steroid receptors make with their natural ligands (FIG. 3). There was an extensive hydrogen bond network involving the A-ring ketone of deoxycorticosterone, Q776 and R817 of MR, and several water molecules that firmly locked the A-ring of the steroid in place. Specific to MR, there was a water-mediated hydrogen bond between Q776 and S810. In AR, GR, and PR, S810 is replaced by a methionine so this interaction is not possible.

Around the deoxycorticosterone D-ring, both the ketone attached to C20 and the hydroxyl attached to C21 were positioned to make hydrogen bonds to Thr945. Thr945 is conserved in GR and PR, but is a leucine in AR.

Deoxycorticosterone binding does not take advantage of the MR specific serine 810, located near the B-ring. Better selectivity over other steroid receptors could be potentially obtained by ligands that interact directly with this residue.

IX.D. Characterization of Interactions between MR and Aldosterone. and Comiarison to other Steroid Receptors with their Cognate Ligands

The structure of MR (C808S) with its natural ligand aldosterone was also determined (Table 4). The protein also crystallized in the space group C222₁, with a=92.49, b=173.25, c=42.19, with one molecule in the asymmetric unit. Crystals diffracted to 1.95 Å at the APS/IMCA beam line 17BM. The final model was refined to R=21.4%, Rfree=23.5%, and contained MR residues 727-908, and 914-983, one molecule of aldosterone, one molecule each of glycerol and β-octyl-glucoside, and 144 water molecules. Overall, the MR(C808S)/aldosterone complex was very similar to the native MR/deoxycorticosterone complex, with bound aldosterone in a fully enclosed pocket contacting residues in helices 3, 4, 5, 6, 7, and 11, and the turn.

MR made many of the same interactions with aldosterone as it did with deoxycorticosterone. There was an extensive hydrogen bond network involving the A-ring ketone of aldosterone, Gln776 and Arg817 of MR, and several water molecules that firmly locked the A-ring of the steroid in place. Specific to MR, there was also a water-mediated hydrogen bond between Gln776 and Ser810. The major difference between deoxycorticosterone and aldosterone was that adjacent to the D-ring, the C-18-OH from aldosterone made a hydrogen bond to Asn770, and both the ketone attached to C20 and the hydroxyl attached to C21 were positioned to make hydrogen bonds to Thr945. Asn770 is conserved in AR, GR, and PR, but Thr945 is conserved in AR and PR, but is a leucine in AR. Aldosterone is selective for MR, and it also does not take advantage of the MR specific serine 810, located near the B-ring.

IX.E. Structural Mechanism of Improving Protein Solubility or Ligand Binding Properties by Mutation of MR LBD and Structural Characteristics and Activation of Mutant MR bound to Agonists

Mutation of select residues of a protein can facilitate greater ease of crystallization of the protein, by for example, increasing the solubility of the protein or improving ligand binding properties. Residues are selected for mutations that do not alter significantly the structure of the protein or its interactions with a ligand. One method useful with the presently disclosed subject matter is to selectively mutate one or more native residues with residues that are less hydrophobic than the native residues. One of skill in the art will recognize the utility of related strategies for mutating residues to increase protein solubility, ligand binding and/or crystallization properties, and these strategies are intended to be included with the presently disclosed subject matter.

Substantial amounts of soluble protein were obtained with both the C808S mutant and the C808S/S810L mutant, with reported partial agonists and prompted comparing the structures of the identical ligand in both forms of the protein. Although the single (C808S) and double (C808S, S810L) MR mutants provided soluble protein/ligand complexes for ligands with potencies ranging from single digit to hundreds of nanomolar, it was necessary to verify that structures obtained using the mutant protein were predictive of compound binding to the native protein. The applicants compared the structures of the native and two mutant MR bound to steroids with varying degrees of potency. The structure of native MR was obtained with the potent agonist deoxycorticosterone.

The structure of the MR C808S,S810L double mutant was also obtained as a complex with progesterone and compared to the same structure obtained with the single C808S mutant. The two complexes crystallized in the same space group, facilitating a direct comparison of the protein conformation. As shown in FIG. 6A, the two versions of the protein have almost identical conformations. Overall, the root mean square (rms.) deviation between equivalent Cα atoms of the two mutants is 0.20 Å, which is slightly larger than the deviation between non-crystallographically related subunits (0.15 Å). The conformation of residues within the binding pocket is also nearly identical between the two forms of the protein (FIG. 6B). In the single mutant, Ser810 binds a water molecule that occupies a hydrophobic pocket adjacent to A773, Q776, M777, W806, M807, and the ligand (FIG. 6B). In the double mutant, the side chain of the L810 occupies this pocket instead of the water.

The double mutant also provides large amounts of soluble protein with other partial agonists as well, including the anti-hypertensive agent spironolactone. Spironolactone is a modified steroid that inhibits the effects of aldosterone. The chemical structure of spironolactone is shown below. Not surprisingly, spironolactone packed into the active site in the same manner as other steroid ligands (FIGS. 7A and 7B). Spironolactone, like progesterone, makes a shorter hydrogen bond with R817 (2.8 Å) than with Q776 (3.3 Å), in contrast to aldosterone, which makes 3.0 Å bonds to both residues. The lactone ring potentially makes weak hydrogen bonds to N770 and T945. The thioester off C7 does not make any interactions with the protein. In fact, the ligand density ends after the sulfur. This result is not unexpected as many spironolactone derivatives, such as canrenone, are the result of modifications of the parent spironolactone. These derivatives may still retain activity. There was no perceptible movement of the AF2 helix in the MR/spironolactone complex. Spironolactone does not impinge on L960, the AF2 residue that caps the binding pocket. The most relevant contact observed is between the lactone ketone oxygen and the side chain of F956, which connects helix 11 to the AF2. At 2.8 Å, spironolactone is closer to this residue than any other compound by ˜0.6 Å.

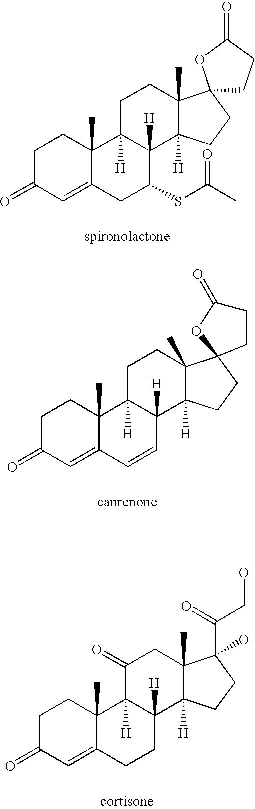

The double mutant also allowed examination of MR's discrimination between cortisone and cortisol, which differ only by the substituents on Cll. Although structurally very similar these compounds have greater than a 700 fold difference in IC50s in the wild type MR in transactivation assays. However, as shown in Table 1, the IC50s are nearly equivalent when the MR double mutant is tested in the same assay. Cortisol has a hydroxyl attached to C11, where cortisone has a ketone. Presented herein is the structure of cortisone, the more weakly binding species in the wild type receptor. The structure suggests the discrimination between cortisol, which has a hydrogen bond donor on C11, and cortisone, which has a hydrogen bond acceptor on C11, reflects the preferred orientation of the Asn770 side chain. The N6 of Asn770 makes an internal hydrogen bond with the carbonyl of Glu955, leaving the 06 facing towards the ligand. Thus, the ligand interacts better with the side chain of Asn770 if it has a hydrogen bond-donor close to this residue. The additional hydrophobic interactions gained between cortisone and the ligand pocket of the MR double mutant (provided by the S810L mutation) are enough to overcome the need for Asn770 to act as a hydrogen bond donor to the cortisone C-11 ketone.

To better understand the mechanism of activating MR, and to compare the native to the single mutant, two structures of MR(C808S) bound to deoxycorticosterone were obtained, both alone and as a complex with a peptide containing residues 732-756 of the nuclear receptor co-activator TIF2, and a structure of MR C808S bound to the natural hormone aldosterone. Although the protein crystallized differently in the presence and absence of co-activator peptide (Table 3 and Table 4 in the Examples below), there was very little difference in the overall structure of the receptor or in the binding pocket (FIG. 4). Presumably the conformational variability of MR in the complexes was diminished because the N-terminal extension's interaction with neighboring molecules served as a surrogate for the co-activator peptide. However, this tendency of MR to crystallize with a “built-in co-activator” facilitated the crystallization and structural determination of steroid partial agonists in the full agonist conformation.

MR structures were also determined with the partial agonist progesterone. Progesterone does not bind adequately to native MR to easily obtain sufficient protein for crystallization trials, however, with the MR C808S mutant protein expression levels are significantly higher and the structure was readily determined. Although the MR(C808S)/progesterone complex crystallized in a different space group (P2₁2₁2₁) than MR/deoxycorticosterone, MR(C808S)/deoxycorticosterone, or MR(C808S)/aldosterone (C2221), the two space groups are related by a crystallographic two-fold and the nearly identical unit cell parameters bear out that the two crystal forms are related. Not surprisingly, the two molecules in the asymmetric unit of the MR/progesterone complex are both similar to each other, and to the MR/deoxycorticosterone complex. As shown in FIG. 5A, comparison of the MR/deoxycorticosterone and MR(C808S)progesterone complexes shows that the overall fold of the proteins are very similar. The residue conformations around the binding pocket are also similar (FIG. 5B). In fact, the major difference between the two complexes is the hydrogen bonding between the protein and the steroid D-ring substituents. In addition to the required A-ring hydrogen bond network, deoxycorticosterone makes two strong hydrogen bonds to T945, and aldosterone makes two strong hydrogen bonds to T945 and N770. Additionally, progesterone's intermolecular hydrogen bond to T945 is competing with intramolecular hydrogen bonds to the amide oxygens of F941 and C942. This competition is not visible in the aldosterone and deoxycorticosterone structures. It appears that the activation of MR is potentially modulated by the strength of the hydrogen bonds between the ligand and T945 and N770 of MR.

IX.F. Generation of Easily-Solved NR, SR and MR Crystals

The present subject matter discloses a substantially pure MR LBD polypeptide in crystalline form. In some embodiments, exemplified in the Figures and Examples, MR is crystallized with bound ligand. Crystals can be formed from NR, SR and MR LBD polypeptides that are usually expressed by cell culture, such as E. coli, Bromo- and iodo-substitutions can be included during the preparation of crystal forms and can act as heavy atom substitutions in MR ligands and crystals of NRs, SRs and MRs. This method can be advantageous for the phasing of the crystal, which is a crucial, and sometimes limiting, step in solving the three-dimensional structure of a crystallized entity. Thus, the need for generating the heavy metal derivatives traditionally employed in crystallography can be eliminated. After the three-dimensional structure of an NR, SR or MR, or an NR, SR or MR LBD with or without a ligand bound is determined, the resultant three-dimensional structure can be used in computational methods to design synthetic ligands for NR, SR or MR and for other NR, SR or MR polypeptides. Further activity structure relationships can be determined through routine testing, using assays disclosed herein and known in the art.

X. Uses of NR, SR and MR Crystals and the Three-Dimensional Structure of the Ligand Bindinq Domain of MR

The solved crystal structure of the presently disclosed subject matter is useful in the design of modulators of activity mediated by the mineralocorticoid receptor and by other nuclear receptors. Evaluation of the available sequence data shows that MR is particularly similar to GR, PR and AR. The MR LBD has approximately 56%, 55% and 51% sequence identity to the GR, PR and AR LBDs, respectively.

The present MR X-ray structure can also be used as a surrogate to build models for targets where no X-ray structure is available, such as with GRβ. Indeed, a model for MR using the available X-ray structures of GRα, PR and/or AR as templates was built and used by the present co-inventors to obtain a starting model for the molecular replacement calculation used in solving the X-ray structure of MR disclosed herein. These models will be less accurate than X-ray structures, but can help in the design of compounds targeted for GRβ, for example. Also, these models can aid the design of compounds to selectively modulate any desired subset of GRα, GRβ, MR, PR, AR and other related nuclear receptors.

X.A. Design and Development of NR, SR and MR Modulators

The presently disclosed subject matter, particularly the computational methods, can be used to design drugs for a variety of nuclear receptors, such as receptors for glucocorticoids (GRs), and rogens (ARs), mineralocorticoids (M Rs), progestins (PRs), estrogens (ERs), thyroid hormones (TRs), vitamin D (VDRs), retinoid (RARs and RXRs) and peroxisomal proliferators (PPARs). The subject matter disclosed herein can also be applied to the “orphan receptors,” as they are structurally homologous in terms of modular domains and primary structure to classic nuclear receptors, such as steroid and thyroid receptors. The amino acid homologies of orphan receptors with other nuclear receptors ranges from very low (<15%) to in the range of 35% when compared to rat RARA and human TRP receptors, for example.

The knowledge of the structure of the MR LBD, as disclosed herein, provides a tool for investigating the mechanism of action of MR and other NR, SR and MR polypeptides in a subject. For example, various computer-modeling programs, as described herein, can predict the binding of various ligand molecules to the LBD of GRβ, or another steroid receptor or, more generally, a nuclear receptor. Upon discovering that such binding in fact takes place, knowledge of the protein structure then allows design and synthesis of small molecules that mimic the functional binding of the ligand to the LBD of MR, and to the LBDs of other polypeptides. This is the method of “rational” drug design, further described herein.

Use of the isolated and purified MR crystalline structure in rational drug design is thus provided in accordance with the presently disclosed subject matter. Additional rational drug design techniques are described in U.S. Pat. Nos. 5,834,228 and 5,872,011, incorporated herein by reference in their entirety.

Thus, in addition to the compounds described herein, other sterically similar compounds can be formulated to interact with the key structural regions of an NR, SR or MR in general, or of MR in particular. The generation of a structural functional equivalent can be achieved by the techniques of modeling and chemical design known to those of skill in the art and described herein. It will be understood that all such sterically similar constructs fall within the scope of the presently disclosed subject matter.

X.A.1. Rational Drug Design

The three-dimensional structure of ligand-binding MR is unprecedented and will greatly aid in the development of new synthetic ligands for NR, SR, and MR polypeptides, such as MR agonists and antagonists, including those that bind exclusively to MR. In addition, NRs, SRs, GRs and MRs are well suited to modern methods, including three-dimensional structure elucidation and combinatorial chemistry, such as those disclosed in U.S. Pat. Nos. 5,463,564, and 6,236,946 incorporated herein by reference. Structure determination using X-ray crystallography is possible because of the solubility properties of NRs, SRs, GRs and MRs. Computer programs that use crystallography data when practicing the presently disclosed subject matter will enable the rational design of ligands to these receptors.

Programs such as RASMOL (Biomolecular Structures Group, Glaxo Wellcome Research & Development Stevenage, Hertfordshire, UK Version 2.6, August 1995, Version 2.6.4, December 1998, © Roger Sayle 1992-1999) and Protein Explorer (Version 1.87, July 3, 2001, © Eric Martz, 2001 and available online at http://www.umass.edu/microbio/chime/explorer/index.htm) can be used with the atomic structural coordinates from crystals generated by practicing the subject matter disclosed herein or used to practice the disclosed subject matter by generating three-dimensional models and/or determining the structures involved in ligand binding. Computer programs such as those sold under the registered trademark INSIGHT II® and the programs GRASP™ (Nicholls et al., (1991) Proteins 11: 281) and SYBYL™ (available from Tripos, Inc. of St. Louis, Mo.) allow for further manipulations and the ability to introduce new structures. In addition, high throughput binding and bioactivity assays can be devised using purified recombinant protein and modern reporter gene transcription assays known to those of skill in the art in order to refine the activity of a designed ligand.

A method of identifying modulators of the activity of an MR polypeptide using rational drug design is thus provided in accordance with the presently disclosed subject matter. The method comprises designing a potential modulator for an MR polypeptide that will form non-covalent interactions with amino acids in the ligand binding pocket based upon the crystalline structure of the MR LBD polypeptide; synthesizing the modulator; and determining whether the potential modulator modulates the activity of the MR polypeptide. Preferably, the MR polypeptide comprises the amino acid sequence of any of SEQ ID NOs:2, 4, 6, 8 and 10. The method can further comprise a crystalline structure having the atomic structure coordinates shown in any of Tables 6-13. The crystalline structure further includes a ligand and a peptide bound to the MR LBD polypeptide. The ligand can be a steroid, such as deoxycorticosterone or aldosterone, and the peptide is a fragment of a co-activator, such as TIF2. The determination of whether the modulator modulates the biological activity of an MR polypeptide is made in accordance with the screening methods disclosed herein, or by other screening methods known to those of skill in the art. Modulators can be synthesized using techniques known to those of ordinary skill in the art.

In an alternative embodiment, a method of designing a modulator of an MR polypeptide in accordance with the subject matter herein is disclosed comprising: (a) selecting a candidate MR ligand; (b) determining which amino acid or amino acids of an MR polypeptide interact with the ligand using a three-dimensional model of a crystallized MR LBD; (c) identifying in a biological assay for MR activity a degree to which the ligand modulates the activity of the MR polypeptide; (d) selecting a chemical modification of the ligand wherein the interaction between the amino acids of the MR polypeptide and the ligand is predicted to be modulated by the chemical modification; (e) synthesizing a chemical compound with the selected chemical modification to form a modified ligand; (f) contacting the modified ligand with the MR polypeptide; (g) identifying in a biological assay for MR activity a degree to which the modified ligand modulates the biological activity of the MR polypeptide; and (h) comparing the biological activity of the MR polypeptide in the presence of modified ligand with the biological activity of the MR polypeptide in the presence of the unmodified ligand, whereby a modulator of an MR polypeptide is designed.

An additional method of designing modulators of an MR or an MR LBD can comprise: (a) determining which amino acid or amino acids of an MR LBD interacts with a first chemical moiety (at least one) of the ligand using a three dimensional model of a crystallized protein comprising an MR LBD in complex with a bound ligand and a co-activator; and (b) selecting one or more chemical modifications of the first chemical moiety to produce a second chemical moiety with a structure to either decrease or increase an interaction between the interacting amino acid and the second chemical moiety compared to the interaction between the interacting amino acid and the first chemical moiety. This is a general strategy only, however, and variations on this disclosed protocol would be apparent to those of skill in the art upon consideration of the present disclosure.

Once a candidate modulator is synthesized as described herein and as will be known to those of skill in the art upon contemplation of the subject matter disclosed herein, it can be tested using assays to establish its activity as an agonist, partial agonist or antagonist, and affinity, as described herein. After such testing, a candidate modulator can be further refined by generating LBD crystals with the candidate modulator bound to the LBD. The structure of the candidate modulator can then be further refined using the chemical modification methods described herein for three dimensional models to improve the activity or affinity of the candidate modulator and make second generation modulators with improved properties, such as that of a super agonist or antagonist, as described herein.

X.A.2. Methods for Using the MR LBD Structural Coordinates For Molecular Design

For the first time, the presently disclosed subject matter permits the use of molecular design techniques to design, select and synthesize chemical entities and compounds, including modulatory compounds, capable of binding to the ligand binding pocket or an accessory binding site of an MR and an MR LBD, in whole or in part. Correspondingly, the present subject matter also provides for the application of similar techniques in the design of modulators of any NR, SR or MR polypeptide.

In accordance with a preferred embodiment, the structure coordinates of a crystalline MR LBD can be used to design compounds that bind to an MR LBD and alter the properties of an MR LBD (for example, the dimerization ability, ligand binding ability or effect on transcription) in different ways. One aspect of the presently disclosed subject matter provides for the design of compounds that can compete with natural or engineered ligands of an MR polypeptide by binding to all, or a portion of, the binding sites on an MR LBD. The present subject matter also provides for the design of compounds that can bind to all, or a portion of, an accessory binding site on an MR that is already binding a ligand. Similarly, non-competitive agonists/ligands that bind to and modulate MR LBD activity, whether or not it is bound to another chemical entity, and partial agonists and antagonists can be designed using the MR LBD structure coordinates disclosed herein.

A second design approach is to probe an MR or an MR LBD crystal with molecules comprising a variety of different chemical entities to determine optimal sites for interaction between candidate MR or MR LBD modulators and the polypeptide. For example, high resolution X-ray diffraction data collected from crystals saturated with solvent allows the determination of the site where each type of solvent molecule adheres. Small molecules that bind tightly to those sites can then be designed and synthesized and tested for their MR modulator activity. Representative designs are also disclosed in published PCT application WO 99/26966.

Once a computationally-designed ligand is synthesized using the methods disclosed herein or other methods known to those of skill in the art, assays can be used to establish its efficacy of the ligand as a modulator of MR activity. After such assays, the ligands can be further refined by generating intact MR or MR LBD crystals with a ligand bound to the LBD. The structure of the ligand can then be further refined using the chemical modification methods described herein and known to those of skill in the art, in order to improve the modulation activity or the binding affinity of the ligand. This process can lead to second generation ligands with improved properties.

Ligands also can be selected that modulate MR responsive gene transcription by the method of altering the interaction of co-activators and co-repressors with their cognate MR. For example, agonistic ligands can be selected that block or dissociate a co-repressor from interacting with an MR, and/or that promote binding or association of a co-activator. Antagonistic ligands can be selected that block co-activator interaction and/or promote co-repressor interaction with a target receptor. Selection can be done via binding assays that screen for designed ligands having the desired modulatory properties. Preferably, interactions of an MR polypeptide are targeted. A suitable assay for screening that can be employed, mutatis mutandis with the present subject matter, is described in Oberfield, J. L., et al., Proc Natl Acad Sci U S A. (1999) May 25; 96(11):6102-6, incorporated herein in its entirety by reference. Other examples of suitable screening assays for MR function include an in vitro peptide binding assay representing ligand-induced interaction with coactivator (Zhou, et al., (1998) Mol. Endocrinol. 12: 1594-1604; Parks et al., (1999) Science 284: 1365-1368) or a cell-based reporter assay related to transcription from a GRE (reviewed in Jenkins et al., (2001) Trends Endocrinol. Metab. 12: 122-126) or a cell-based reporter assay related to repression of genes driven via NF-κB. DeBosscher et al., (2000) Proc Natl Acad Sci U S A. 97: 3919-3924.

X.A.3. Methods of Designing NR. SR or MR LBD Modulator Compounds

Knowledge of the three-dimensional structure of the MR LBD complex of the subject matter disclosed herein can facilitate a general model for modulator (e.g. agonist, partial agonist, antagonist and partial antagonist) design. Other ligand-receptor complexes belonging to the nuclear receptor superfamily can have a ligand binding pocket similar to that of MR and therefore the present subject matter can be employed in agonist/antagonist design for other members of the nuclear receptor superfamily and the steroid receptor subfamily. Examples of suitable receptors include those of the NR superfamily and those of the SR subfamily.

The design of candidate substances, also referred to as “compounds” or “candidate compounds”, that bind to or inhibit NR, SR or MR LBD-mediated activity according to the present subject matter generally involves consideration of two factors. First, the compound must be capable of physically and structurally associating with an NR, SR or MR LBD. Non-covalent molecular interactions important in the association of an NR, SR or MR LBD with its substrate include hydrogen bonding, van der Waals interactions and hydrophobic interactions.

The interaction between an atom of a LBD amino acid and an atom of an LBD ligand can be made by any force or attraction described in nature. Usually the interaction between the atom of the amino acid and the ligand will be the result of a hydrogen bonding interaction, charge interaction, hydrophobic interaction, van der Waals interaction or dipole interaction. In the case of the hydrophobic interaction it is recognized that this is not a per se interaction between the amino acid and ligand, but rather the usual result, in part, of the repulsion of water or other hydrophilic group from a hydrophobic surface. Reducing or enhancing the interaction of the LBD and a ligand can be measured by calculating or testing binding energies, computationally or using thermodynamic or kinetic methods as known in the art.

Second, the compound must be able to assume a conformation that allows it to associate with an NR, SR or MR LBD. Although certain portions of the compound will not directly participate in this association with an NR, SR or MR LBD, those portions can still influence the overall conformation of the molecule. This, in turn, can have a significant impact on potency. Such conformational requirements include the overall three-dimensional structure and orientation of the chemical entity or compound in relation to all or a portion of the binding site, e.g., the ligand binding pocket or an accessory binding site of an NR, SR or MR LBD, or the spacing between functional groups of a compound comprising several chemical entities that directly interact with an NR, SR or MR LBD.

Chemical modifications will often enhance or reduce interactions of an atom of an LBD amino acid and an atom of an LBD ligand. Steric hindrance can be a common means of changing the interaction of an LBD binding pocket with an activation domain. Chemical modifications are preferably introduced at C—H, C— and C—OH positions in a ligand, where the carbon is part of the ligand structure that remains the same after modification is complete. In the case of C—H, C could have 1, 2 or 3 hydrogens, but usually only one hydrogen will be replaced. The H or OH can be removed after modification is complete and replaced with a desired chemical moiety.

The potential modulatory or binding effect of a chemical compound on an NR, SR or MR LBD can be analyzed prior to its actual synthesis and testing by the use of computer modeling techniques that employ the coordinates of a crystalline MR LBD polypeptide of the presently disclosed subject matter. If the theoretical structure of the given compound suggests insufficient interaction and association between it and an NR, SR or MR LBD, synthesis and testing of the compound is obviated. However, if computer modeling indicates a strong interaction, the molecule can then be synthesized and tested for its ability to bind and modulate the activity of an NR, SR or MR LBD. In this manner, synthesis of unproductive or inoperative compounds can be avoided.

A modulatory or other binding compound of an NR, SR or MR LBD polypeptide (preferably an MR LBD) can be computationally evaluated and designed via a series of steps in which chemical entities or fragments are screened and selected for their ability to associate with an individual binding site or other area of a crystalline MR LBD polypeptide of the subject matter disclosed herein and to interact with the amino acids disposed in the binding sites.

Interacting amino acids forming contacts with a ligand and the atoms of the interacting amino acids are usually 2 to 4 angstroms away from the center of the atoms of the ligand. Generally these distances are determined by computer as discussed herein and in McRee (McRee, (1993) Practical Protein Crystallography, Academic Press, New York), however distances can be determined manually once the three dimensional model is made. More commonly, the atoms of the ligand and the atoms of interacting amino acids are 3 to 4 angstroms apart. A ligand can also interact with distant amino acids, after chemical modification of the ligand to create a new ligand. Distant amino acids are generally not in contact with the ligand before chemical modification. A chemical modification can change the structure of the ligand to make as new ligand that interacts with a distant amino acid usually at least 4.5 angstroms away from the ligand. Often distant amino acids will not line the surface of the binding cavity for the ligand, as they are too far away from the ligand to be part of a pocket or surface of the binding cavity.

A variety of methods can be used to screen chemical entities or fragments for their ability to associate with an NR, SR or MR LBD and, more particularly, with the individual binding sites of an NR, SR or MR LBD, such as ligand binding pocket or an accessory binding site. This process can begin by visual inspection of, for example, the ligand binding pocket on a computer screen based on the MR LBD atomic coordinates in Tables 6-13, as described herein. Selected fragments or chemical entities can then be positioned in a variety of orientations, or docked, within an individual binding site of an MR LBD as defined herein above. Docking can be accomplished using software programs such as those available under the tradenames QUANTA™ (Molecular Simulations Inc., San Diego, Calif.) and SYBYL™ (Tripos, Inc., St. Louis, Mo.), followed by energy minimization and molecular dynamics with standard molecular mechanics forcefields, such as CHARM (Brooks et al., (1983) J. Comp. Chem., 8: 132) and AMBER 5 (Case et al., (1997), AMBER 5, University of California, San Francisco; Pearlman et al., (1995) Comput. Phys. Commun. 91:141).

Specialized computer programs can also assist in the process of selecting fragments or chemical entities. These include:

1. GRID™ program, version 17 (Goodford, (1985) J. Med. Chem. 28: 849-57), which is available from Molecular Discovery Ltd., Oxford, UK;

2. MCSS™ program (Miranker & Karplus, (1991) Proteins 11: 29-34), which is available from Molecular Simulations, Inc., San Diego, Calif.;

3. AUTODOCK™ 3.0 program (Goodsell & Olsen, (1990) Proteins 8: 195-202), which is available from the Scripps Research Institute, La Jolla, California;

4. DOCK™ 4.0 program (Kuntz et al., (1992) J. Mol. Biol. 161: 269-88), which is available from the University of California, San Francisco, Calif.;

5. FLEX-X™ program (See, Rarey et al., (1996) J. Comput. Aid. Mol. Des. 10:41-54), which is available from Tripos, Inc., St. Louis, Mo.;

6. MVP program (Lambert, (1997) in Practical Application of Computer-Aided Drug Design, (Charifson, ed.) Marcel-Dekker, New York, pp. 243-303); and

7. LUDI™ program (Bohm, (1992) J. Comput. Aid. Mol. Des., 6: 61-78), which is available from Molecular Simulations, Inc., San Diego, Calif.

Once suitable chemical entities or fragments have been selected, they can be assembled into a single compound or modulator. Assembly can proceed by visual inspection of the relationship of the fragments to each other on the three-dimensional image displayed on a computer screen in relation to the structure coordinates of an MR LBD. Manual model building using software such as QUANTA™ or SYBYL™ typically follows.

Useful programs to aid one of ordinary skill in the art in connecting the individual chemical entities or fragments include:

1. CAVEAT™ program (Bartlett et al., (1989) Special Pub., Royal Chem. Soc. 78: 182-96), which is available from the University of California, Berkeley, Calif.;

2. 3D Database systems, such as MACCS-3DTM system program, which is available from MDL Information Systems, San Leandro, Calif. This area is reviewed in Martin, (1992) J. Med. Chem. 35: 2145-54; and

3. HOOK™ program (Eisen et al., (1994). Proteins 19: 199-221), which is available from Molecular Simulations, Inc., San Diego, Calif.

Instead of proceeding to build an MR LBD modulator in a step-wise fashion one fragment or chemical entity at a time as described above, modulatory or other binding compounds can be designed as a whole or de novo using the structural coordinates of a crystalline MR LBD polypeptide of the subject matter disclosed herein and either an empty binding site or optionally including some portion(s) of a known modulator(s). Applicable methods can employ the following software programs:

1. LUDI™ program (Bohm, (1992) J. Comput. Aid. Mol. Des., 6: 61-78), which is available from Molecular Simulations, Inc., San Diego, Calif.;

2. LEGEND™ program (Nishibata & Itai, (1991) Tetrahedron 47: 8985); and

3. LEAPFROG™, which is available from Tripos Associates, St. Louis, Mo.

Other molecular modeling techniques can also be employed in accordance with the subject matter disclosed herein. See. e.g., Cohen et al., (1990) J. Med. Chem. 33: 883-94. See also, Navia & Murcko, (1992) Curr. Opin. Struc. Biol. 2: 202-10; U.S. Pat. No. 6,008,033, herein incorporated by reference.

Once a compound has been designed or selected by the above methods, the efficiency with which that compound can bind to an NR, SR or MR LBD can be tested and optimized by computational evaluation. By way of particular example, a compound that has been designed or selected to function as an NR, SR or MR LBD modulator should also preferably traverse a volume not overlapping that occupied by the binding site when it is bound to its native ligand. Additionally, an effective NR, SR or MR LBD modulator should preferably demonstrate a relatively small difference in energy between its bound and free states (i.e., a small deformation energy of binding). Thus, the most efficient NR, SR and MR LBD modulators should preferably be designed with a deformation energy of binding of not greater than about 10 kcal/mole, and preferably, not greater than 7 kcal/mole. It is possible for NR, SR and MR LBD modulators to interact with the polypeptide in more than one conformation that is similar in overall binding energy. In those cases, the deformation energy of binding is taken to be the difference between the energy of the free compound and the average energy of the conformations observed when the modulator binds to the polypeptide.

A compound designed or selected as binding to an NR, SR or MR polypeptide (in some embodiments an MR LBD polypeptide) can be further computationally optimized so that in its bound state it would preferably lack repulsive electrostatic interaction with the target polypeptide. Such non-complementary (e.g., electrostatic) interactions include repulsive charge-charge, dipole-dipole and charge-dipole interactions. Specifically, the sum of all electrostatic interactions between the modulator and the polypeptide when the modulator is bound to an NR, SR or MR LBD preferably make a neutral or favorable contribution to the enthalpy of binding.

Specific computer software is available in the art to evaluate compound deformation energy and electrostatic interaction. Examples of programs designed for such uses include:

1. GAUSSIAN 98™, which is available from Gaussian, Inc., Pittsburgh, Pa.;

2. AMBER™ program, version 6.0, which is available from the University of California at San Francisco;

3. QUANTA™ program, which is available from Molecular Simulations, Inc., San Diego, Calif.;

4. CHARM® program, which is available from Molecular Simulations, Inc., San Diego, Calif.; and

5. INSIGHT II® program, which is available from Molecular Simulations, Inc., San Diego, Calif.

These programs can be implemented using a suitable computer system. Other hardware systems and software packages will be apparent to those skilled in the art after review of the disclosure presented herein.

Once an NR, SR or MR LBD modulating compound has been optimally selected or designed, as described above, substitutions can then be made in some of its atoms or side groups in order to improve or modify its binding properties. Generally, initial substitutions are conservative, i.e., the replacement group will have approximately the same size, shape, hydrophobicity and charge as the original group. It should, of course, be understood that components known in the art to alter conformation are preferably avoided. Such substituted chemical compounds can then be analyzed for efficiency of fit to an NR, SR or MR LBD binding site using the same computer-based approaches described in detail above.

X.B. Distinguishing Between NRs and Subtvpes

The presently disclosed subject matter is also applicable to generating new synthetic ligands to distinguish nuclear receptors and subtypes. As described herein, modulators can be generated that distinguish between subtypes, thereby allowing the generation of either tissue specific or function specific synthetic ligands.

A method of identifying an NR modulator that selectively modulates the biological activity of one NR compared to MR is also disclosed. In one embodiment, the method comprises: (a) providing an atomic structure coordinate set describing an MR ligand binding domain structure and at least one other atomic structure coordinate set describing an NR ligand binding domain, each ligand binding domain comprising a ligand binding site; (b) comparing the atomic structure coordinate sets to identify at least one difference between the sets; (c) designing a candidate ligand predicted to interact with the difference of step (b); (d) synthesizing the candidate ligand; and (e) testing the synthesized candidate ligand for an ability to selectively modulate an NR as compared to MR, whereby an NR modulator that selectively modulates the biological activity NR compared to MR is identified.

Preferably, the MR atomic structure coordinate set is the atomic structure coordinate set shown in any of Tables 6-13. Optionally, the NR is selected from the group consisting of MR, PR, AR, GRα, GRβ and isoforms thereof that have ligands that also bind MR.

X.C. Method of Screening for Chemical and Biological Modulators of the Biological Activity of an MR

A candidate substance identified according to a screening assay of the presently disclosed subject matter has an ability to modulate the biological activity of an MR or an MR LBD polypeptide. In a preferred embodiment, such a candidate compound can have utility in the treatment of disorders and/or conditions and/or biological events associated with the biological activity of an MR or an MR LBD polypeptide, including transcription modulation.

In a cell-free system, the method comprises the steps of establishing a control system comprising an MR polypeptide and a ligand which is capable of binding to the polypeptide; establishing a test system comprising an MR polypeptide, the ligand, and a candidate compound; and determining whether the candidate compound modulates the activity of the polypeptide by comparison of the test and control systems. A representative ligand can comprise deoxycorticosterone, aldosterone, or other small molecules, and in some embodiments, the biological activity or property screened can include binding affinity or transcription regulation. The MR polypeptide can be in soluble or crystalline form.

In another embodiment, a soluble or a crystalline form of an MR polypeptide or a catalytic or immunogenic fragment or oligopeptide thereof, can be used for screening libraries of compounds in any of a variety of drug screening techniques. The fragment employed in such a screening technique can be affixed to a solid support. The formation of binding complexes, between a soluble or a crystalline MR polypeptide and the agent being tested, will be detected. In a preferred embodiment, the soluble or crystalline MR polypeptide has an amino acid sequence of SEQ ID NO: 2. When an MR LBD polypeptide is employed, a preferred embodiment will include a soluble or a crystalline MR polypeptide having the amino acid sequence of any of SEQ ID NOs:4, 6, 8 or 10.

Another technique for drug screening which can be used provides for high throughput screening of compounds having suitable binding affinity to the protein of interest as described in published PCT application WO 84/03564, herein incorporated by reference. In this method, as applied to a soluble or crystalline polypeptide of the presently disclosed subject matter, large numbers of different small test compounds are synthesized on a solid substrate, such as plastic pins or some other surface. The test compounds are reacted with the soluble or crystalline polypeptide, or fragments thereof. Bound polypeptide is then detected by methods known to those of skill in the art. The soluble or crystalline polypeptide can also be placed directly onto plates for use in the aforementioned drug screening techniques.

In yet another embodiment, a method of screening for a modulator of an MR or an MR LBD polypeptide comprises: providing a library of test samples; contacting a soluble or a crystalline form of an MR or a soluble or crystalline form of an MR LBD polypeptide with each test sample; detecting an interaction between a test sample and a soluble or a crystalline form of an MR or a soluble or a crystalline form of an MR LBD polypeptide; identifying a test sample that interacts with a soluble or a crystalline form of an NR, SR or MR or a soluble or a crystalline form of an MR LBD polypeptide; and isolating a test sample that interacts with a soluble or a crystalline form of an MR or a soluble or a crystalline form of an MR LBD polypeptide.

In each of the foregoing embodiments, an interaction can be detected spectrophotometrically, radiologically, calorimetrically or immunologically. An interaction between a soluble or a crystalline form of an MR or a soluble or a crystalline form of an MR LBD polypeptide and a test sample can also be quantified using methodology known to those of skill in the art.

In accordance with the subject matter disclosed herein, there is also provided a rapid and high throughput screening method that relies on the methods described above. This screening method comprises separately contacting each of a plurality of substantially identical samples with a soluble or a crystalline form of an NR, SR or MR or a soluble or a crystalline form of an NR, SR or MR LBD and detecting a resulting binding complex. In such a screening method the plurality of samples preferably comprises more than about 10⁴ samples, or more preferably comprises more than about 5×10⁴ samples.

In another embodiment, a method for identifying a substance that modulates MR LBD function is also provided. In a preferred embodiment, the method comprises: (a) isolating an MR polypeptide; (b) exposing the isolated MR polypeptide to a plurality of substances; (c) assaying binding of a substance to the isolated MR polypeptide; and (d) selecting a substance that demonstrates specific binding to the isolated MR LBD polypeptide. By the term “exposing the MR polypeptide to a plurality of substances”, it is meant both in pools and as multiple samples of “discrete” pure substances.

X.D. Method of Identifying Compounds That Inhibit Ligand Binding

In one aspect of the subject matter disclosed herein, an assay method for identifying a compound that inhibits binding of a ligand to an MR polypeptide is disclosed. A ligand, such as deoxycorticosterone or aldosterone (which associates with at least MR), can be used in the assay method as the ligand against which the inhibition by a test compound is gauged. The method comprises (a) incubating an MR polypeptide with a ligand in the presence of a test inhibitor compound; (b) determining an amount of ligand that is bound to the MR polypeptide, wherein decreased binding of ligand to the MR polypeptide in the presence of the test inhibitor compound relative to binding in the absence of the test inhibitor compound is indicative of inhibition; and (c) identifying the test compound as an inhibitor of ligand binding if decreased ligand binding is observed. In some embodiments, the ligand is deoxycorticosterone.

In another aspect of the presently disclosed subject matter, the disclosed assay method can be used in the structural refinement of candidate MR inhibitors. For example, multiple rounds of optimization can be followed by gradual structural changes in a strategy of inhibitor design. A strategy such as this is made possible by the disclosure of the atomic coordinates of the MR LBD.

XI. The Role of the Three-Dimensional Structure of the MR LDB in Solving Additional NR. SR or MR Crvstals

Because polypeptides can crystallize in more than one crystal form, the structural coordinates of an MR LBD, or portions thereof, as provided by the present subject matter, are particularly useful in solving the structure of crystal forms of other NRs, SRs and MRs. The coordinates provided herein can also be used to solve the structure of NRs, SRs or MRs and NR, SR or MR LBD mutants (such as those described in the Sections above), NR, SR or MR LDB co-complexes, or of the crystalline form of any other protein with significant amino acid sequence homology to any functional domain of NR, SR or MR.

XI.A. Determining the Three-Dimensional Structure of a Polyveptide Using the Three-Dimensional Structure of the MR LBD as a Template in Molecular Replacement

One method that can be employed for the purpose of solving additional NR crystal structures is molecular replacement. See generally, Rossmann, ed, (1972) The Molecular Replacement Method, Gordon & Breach, New York. In the molecular replacement method, the unknown crystal structure, whether it is another crystal form of an MR or an MR LBD, (i.e. an MR LBD mutant), or an NR, SR or MR or an NR, SR or MR LBD polypeptide complexed with another compound (a “co-complex”), or the crystal of some other protein with significant amino acid sequence homology to any functional region of the MR LBD, can be determined using the MR LBD structure coordinates provided in any of Tables 6-13. This method provides an accurate structural form for the unknown crystal more quickly and efficiently than attempting to determine such information ab initio.

In addition, in accordance with the subject matter disclosed herein, NR, SR or MR and NR, SR or MR LBD mutants can be crystallized in complex with known modulators. The crystal structures of a series of such complexes can then be solved by molecular replacement and compared with that of the wild-type NR, SR or MR or the wild-type NR, SR or MR LBD. Potential sites for modification within the various binding sites of the enzyme can thus be identified. This information provides an additional tool for determining the most efficient binding interactions, for example, increased hydrophobic interactions, between the MR LBD and a chemical entity or compound.

All of the complexes referred to in the present disclosure can be studied using X-ray diffraction techniques (See, e.g., Blundell & Johnson (1985) Method. Enzymol., 114A & 115B, (Wyckoff et al., eds.), Academic Press; McRee, (1993) Practical Protein Crystallography, Academic Press, New York) and can be refined using computer software, such as the X-PLOR™ program (Brünger, (1992) X-PLOR, Version 3.1. A System for X-ray Crystallography and NMR, Yale University Press, New Haven, Conn.; X-PLOR is available from Molecular Simulations, Inc., San Diego, Calif.) and the XTAL-VIEW program (McRee, (1992) J. Mol. Graphics 10: 44-46; McRee, (1993) Practical Protein Crvstallography, Academic Press, San Diego, California). This information can thus be used to optimize known classes of MR and MR LBD modulators, and more importantly, to design and synthesize novel classes of MR and MR LBD modulators.

### EXAMPLES

The following Examples have been included to illustrate representative modes of the present disclosure. Certain aspects of the following Examples are described in terms of techniques and procedures found or contemplated by the present inventors to work well in the practice of the subject matter disclosed herein. In light of the present disclosure and the general level of skill in the art, those of skill will appreciate that the following Examples are intended to be exemplary only and that numerous changes, modifications and alterations can be employed without departing from the spirit and scope of the presently disclosed subject matter.

### Example 1

### Mutagenesis (C808S and C808S+S810L) of Human MR Ligand Binding Domain (LBD)

Two complimentary oligonucleotides for each desired mutation were constructed. The following sequences represent the oligonucleotides for the Cysteine 808 Serine mutation:

Forward Primer (C808S) (SEQ ID NO:13):
5′ GTA TTC TTG GAT GTC TCT ATC ATC ATT TGC CT 3′
Reverse Primer (C808S) (SEQ ID NO:14):
5′ AGG CAA ATG ATG ATA GAG ACA TCC AAG AAT AC 3′

Another separate mutation was also constructed. The sequences below represent the oligonucleotides for the Cysteine 808 Serine and Serine 810 Leucine combination mutation:

Forward Primer (C808S, S810L) (SEQ ID NO:15):
5′ TCT TGG ATG TCT CTA TTA TCA TTT GCC T 3′
Reverse Primer (C808, S810LS) (SEQ ID NO:16):
5′ AGG CAA ATG ATA ATA GAG ACA TCC AAG A 3′

The underlined bolded letters depict the base changes from the wild type human MR sequence. The MR LBD (amino acids 712-984) (SEQ ID NOs:3-4) previously cloned into the modified 6×HisGST-pET24 vector (Invitrogen, Carlsbad, Calif., USA) was used as the backbone to create the mutants. The procedure used to make the mutation is outlined in the QUICKCHANGE™ Site-Directed Mutagenesis Kit sold by Stratagene, La Jolla, California, USA (Catalog # 200518). Final constructs were sequence verified. A thrombin protease site at the C-terminus of the glutathione S-transferase allows for cleavage of the resultant fusion protein following expression.

The resulting final amino acid sequences for the mutant MR LBDs are below. The underlined, bolded amino acids depict the changes from the wild type human MR sequence.

MR-LBD (712-984) C808S
(SEQ ID NO:6)
APAKEPSVNT ALVPQLSTIS RALTPSPVMV LENIEPEIVY AGYDSSKPDT 50
AENLLSTLNR LAGKQMIQVV KWAKVLPGFK NLPLEDQITL IQYSWMSLSS 100
FALSWRSYKH TNSQFLYFAP DLVFNEEKMH QSAMYELCQG MHQISLQFVR 150
LQLTFEEYTI MKVLLLLSTI PKDGLKSQAA FEEMRTNYIK ELRKMVTKCP 200
NNSGQSWQRF YQLTKLLDSM HDLVSDLLEF CFYTFRESHA LKVEFPAMLV 250
EIISDQLPKV ESGNAKPLYF HRK 273
MR-LBD (712-984) C808S, S810L
(SEQ ID NO:10)
APAKEPSVNT ALVPQLSTIS RALTPSPVMV LENIEPEIVY AGYDSSKPDT 50
AENLLSTLNR LAGKQMIQVV KWAKVLPGFK NLPLEDQITL IQYSWMSLLS 100
FALSWRSYKH TNSQFLYFAP DLVFNEEKMH QSAMYELCQG MHQISLQFVR 150
LQLTFEEYTI MKVLLLLSTI PKDGLKSQAA FEEMRTNYIK ELRKMVTKCP 200
NNSGQSWQRF YQLTKLLDSM HDLVSDLLEF CFYTFRESHA LKVEFPAMLV 250
EIISDQLPKV ESGNAKPLYF HRK 273

### Example 2

### MR C808S Mutation

In an effort to increase expression yields, a cysteine residue located at position 808 in the human MR protein was mutated to serine in accordance with approaches disclosed in Example 1. This position is equivalent in location to amino acid 602 of the human GR. Previous work with GR had demonstrated that a cysteine in that position did not aid expression of that receptor.

This mutation (C808S) led to increased expression of MR in the presence of high affinity ligands and resulted in the solving of multiple crystal structures including MR with the high affinity ligands aldosterone, deoxycorticosterone and progesterone. These structures are summarized in Table 5 of Example 10 below. Only minor differences are seen in the wild type and mutant structure comparisons. Expression of the receptor was completed and the MR LBD was purified as described in the Examples herein below. The protein was then crystallized as described in Example 8 below.

### Example 3

### MR S810L and C808S. S810L Mutations

An S810L mutation was created within the LBD of MR in accordance with approaches disclosed herein below, which led to increased affinities of certain compounds including spironolactone and cortisone (Geller et al., 2000 and Rafestin-Oblin et al., 2003), as described in the Examples herein. A double mutation (C808S, S810L) was also created within the LBD of MR in accordance with approaches disclosed in Example 1.

While bacterial expression levels of the MR-C808S and MR-S810L constructs are markedly different versus the wild type receptor in the presence of the high affinity ligand aldosterone (FIG. 11), expression levels of these constructs in the presence of (10 uM concentration) low affinity ligands (cortisone, spironolactone and canrenone) do not appear to vary significantly from that of the wild type receptor. However, expression of the MR C808S, S810L combination mutant is enhanced even in the presence of the low affinity compounds (FIG. 11). This allowed for subsequent purification of the expressed protein and the first crystal structure of MR with spironolactone, a hypertension drug currently on the market. These structures are summarized in Table 5 below in Example 10. Expression of the receptor was completed and the MR LBD was purified as described in Example 6. The protein was then crystallized as described in Example 8.

### Example 4

### Expression of MR C808S. S810L Mutant in the Presence of Cortisone

MR binds many glucocorticoids such as cortisol and corticosterone with affinities similar to that seen with aldosterone. In fact, because both GR and MR bind cortisol with high affinity and because circulating levels of cortisol range from 100 to 1000 fold of those seen with aldosterone, selectivity within MR target tissues such as the kidneys is obtained by the location of 11 PHSD2 enzyme that converts the high affinity cortisol (low nM binder) to the much weaker ligand cortisone (micromolar binder) (Quinkler & Stewart, 2003). Surprisingly, an increase of expression of the MR double mutant in the presence of the low affinity ligand cortisone is seen (FIG. 11) using the methods described herein above. These data demonstrate high levels of expression of the MR double mutant even in the presence of ligands with low binding affinity for MR.

Expression of the receptor was completed and the MR LBD was purified as described in Example 6. The protein was then crystallized as described in Example 8 herein below.

### Example 5

### Expression of an MR-LBD Polypeptide

BL21(DE3) cells (Novagen/Invitrogen, Inc., Carlsbad, Calif., USA) were transformed with the expression plasmid 6×HisGST-MR(712-984)pET24, 6×HisGST-MR(712-984)C808S pET24 or 6×HisGST-MR(712-984)C808S,S810L pET24 following established protocols. Following overnight incubation at 37° C. a single colony was used to inoculate a 10 ml LB culture containing 50 μg/ml kanamycin (Sigma, St. Louis, Mo., USA). The culture was grown for ˜8 hrs at 37° C. and then a 1 ml aliquot was used to inoculate flasks containing 1 liter CIRCLE GROW™ media (Bio 101, Inc., Vista, Calif., USA) and the required antibiotic. The cells were then grown at 23° C. to an OD600 between 2 and 3 and then cooled to 16-18° C. Following a 30 minute equilibration at that temperature, 10 to 100 μM of the desired ligand was added. Induction of expression was achieved by adding IPTG (BACHEM, Philadelphia, Pa., USA) (final concentration 250 μM) to the cultures. Expression at 16° C. was continued for -24 hrs. Cells were then harvested and frozen at −80° C.

Referring now to FIG. 8, E. coli expression of mutant 6×HisGST-MR(712-984) C808S versus 6×HisGST-wild type MR is shown. Shown are the eluent fractions (soluble nickel resin binding) fractions of wild type MR expressed in the presence of no ligand (Lane 1), 20 μM aldosterone (lane 2) and 20 μM deoxycorticosterone (lane 3). Also shown are the eluent fractions (soluble nickel resin binding) for the 6×HisGST-MR(712-984) C808S mutant expressed in the presence of no ligand (lane 4) 20 μM deoxycorticosterone (lane 5), 20 μM aldosterone (lane 6) and 20 μM dexamethasone (lane 7). The positions of the molecular mass (kDa) markers lane M (94, 67, 43, 30, 20, 14) and of the expressed fusion proteins are indicated to the left and right sides of the panel respectively.

### Example 6

### Purification of an MR-LBD Polyveptide Bound to Ligand

Previously grown E. coli cells containing the protein of interest were resuspended in lysis buffer (50 mM Tris pH=8.0, 150 mM NaCl, 2M urea, and 50 JIM ligand) and lysed by passing 3 times through a Rannie APV Lab 2000 homogenizer (Rannie APV, Copenhagen, Denmark). The lysate was subjected to centrifugation (30 minutes, 20,000g, 4° C.). The cleared supernatant was filtered through a Pall Kleen-Pak filter (Pall Corporation, East Hills, New York, USA) and 50 mM Tris, pH=8.0, containing 150 mM NaCl and 1M imidazole was added to obtain a final imidazole concentration of 50 mM. This lysate was loaded onto a XK-26 column (Pharmacia, Peapack, N.J., USA) packed with Sepharose [Ni²⁺ charged] chelation resin (Pharmacia, Peapack, N.J., USA) pre-equilibrated with lysis buffer supplemented with 50 mM imidazole.

Following loading, the column was washed to baseline absorbance with equilibration buffer. This was followed by a linear (0 to 10%) glycerol and (2M to 0M) urea gradient. For elution, the column was developed with a linear gradient from 50 to 500 mM imidazole in 50 mM Tris pH=8.0, 150 mM NaCl, 10% glycerol and 30 pM ligand. Column fractions of interest were pooled and 500 units of thrombin protease (Amersham Pharmacia Biotech, Piscataway, N.J., USA) were added for the cleavage of the fusion protein. This solution was then dialyzed against 1 liter of 50 mM Tris pH=8.0, 150 mM NaCl, 10% glycerol and 30 μM Ligand for 18 hrs at 4° C. The digested protein sample was filtered and then reloaded onto a fresh Ni⁺⁺ charged column. The cleaved MR-LBD was collected in the flow-through fraction. The protein sample was then diluted 5 fold with 25 mM Hepes pH=7.0, 10% glycerol, 10 mM DTT, 0.5 mM EDTA and 30 μM ligand. Following dilution, the sample was loaded onto 2 sequential pre-equilibrated XK-26 columns (Pharmacia, Peapack, N.J., USA) packed with Poros HQ resin (PerSeptive Biosystems, Framingham, Mass., USA) followed by a column packed with Poros HS resin (PerSeptive Biosystems, Framingham, Mass., USA). Following loading of the sample, the Poros HQ column was detached and the bound MR was eluted from the HS column with a 30-500 mM NaCl gradient. The purified protein was then concentrated to ˜3.5 mg/ml using the JumboSep (Pall Life Sciences, Ann Arbor, Mich., USA) centrifugal filtration devices.

FIGS. 10 and 12 depict purification of the E. coli expressed MR(712-984) C808S bound with aldosterone (FIG. 10) or deoxycorticosterone (FIG. 12) by SDS PAGE.

The final resultant sequences (SEQ ID NO:17 and SEQ ID NO:18) of the purified mutant proteins are below. The first two residues (underlined and bolded) are vector derived and represent the remaining residues of the thrombin cleavage site following digestion.

SEQ ID NO:17
GSAPAKEPSV NTALVPQLST ISPALTPSPV MVLENIEPEI VYAGYDSSKP 50
DTAENLLSTL NRLAGKQMIQ VVKWAKVLPG FKNLPLEDQI TLIQYSWMSL 100
SSFALSWRSY KHTNSQFLYF APDLVFNEEK MHQSAMYELC QGMHQISLQF 150
VRLQLTFEEY TIMKVLLLLS TIPKDGLKSQ AAFEEMRTNY IKELRKMVTK 200
CPNNSGQSWQ RFYQLTKLLD SMHDLVSDLL EFCFYTFRES HALKVEFPAM 250
LVEIISDQLP KVESGNAKPL YFHRK 275
SEQ ID NO:18
GSAPAKEPSV NTALVPQLST ISRALTPSPV MVLENIEPEI VYAGYDSSKP 50
DTAENLLSTL NRLAGKQMIQ VVKWAKVLPG FKNLPLEDQI TLIQYSWMSL 100
LSFALSWRSY KHTNSQFLYF APDLVFNEEK MHQSAMYELC QGMHQISLQF 150
VRLQLTFEEY TIMKVLLLLS TIPKDGLKSQ AAFEEMRTNY IKELRKMVTK 200
CPNNSGQSWQ RFYQLTKLLD SMHDLVSDLL EFCFYTFRES HALKVEFPAM 250
LVEIISDQLP KVESGNAKPL YFHRK 275

### Co-Activator Recruitment bv MR LBD Mutants

Ligand-activated transcriptional regulation of nuclear receptors involves the participation of cofactors that can function as activators or repressors of receptor mediated transcription (Subramaniam et al., 1999; and McKenna & O'Malley, 2002). Because the association of cofactors with NR LBDs is often amplified once ligand is bound to the receptor, co-activator peptide recruitment experiments provide a valuable means to characterize the receptor. Depending on the experimental setup both ligand and co-activator peptide affinities can be determined.

For example, using the 6×HisGST-MR proteins expressed in the presence of ligand, partially purified and then dialyzed extensively, a set of peptide recruitment experiments were conducted. As shown in FIG. 9A, using the MR C808S receptor, recruitment experiments with a co-activator peptide derived from the transcriptional intermediary factor 2 (TIF-2) yield relative ligand affinities of 9.4 nM, 9780.0 nM, 853.7 nM and >10,000 nM for the ligands deoxycorticosterone, cortisone, spironolactone and canrenone, respectively. In contrast, as shown in FIG. 9B, using the MR C808S, S810L protein, the observed affinity for deoxycorticosterone remains nearly the same (7.3 nM). However, the affinities with the normally weak ligands cortisone (453.7 nM), spironolactone (30.9 nM) and canrenone (114.0 nM) are all greatly enhanced with this protein.

This increase in ligand affinity suggests normally weak ligands such as spironolactone, canrenone, and cortisone can aid expression and crystallization of the MR C808S, S810L compared to the native receptors. As FIGS. 9A and 9B show, expression of the MR C808S, S810L protein in the presence of these ligands is greatly enhanced over that seen with the native or MR C808S protein. Furthermore, the crystal structures of the MR C808S, S810L protein bound to spironolactone and cortisone have been determined as described in the Examples below and shown in FIGS. 7A and 7B, respectively. Since there is significant cross binding of many ligands between the SRs then the MR C808S, S810L protein may be used as a surrogate receptor for all SRs.

### Example 8

### Crystallization and Data Collection

MR (C808S) LBD was bound with deoxycorticosterone, deoxycorticosterone and a fragment of the TIF2 peptide (GLN-GLU-PRO-VAL-SER-PRO-LYS-LYS-LYS-GLU-ASN-ALA-LEU-LEU-ARG-TYR-LEU-LEU-ASP-LYS-ASP-ASP-THR-LYS-ASP) (SEQ ID NO:11), aldosterone, or progesterone. The MR protein was concentrated to ˜4 mgs/ml. The protein buffer comprised:

25 mM Hepes 7.0

˜190 mM NaCl

10% glycerol

10 mM DTT

0.5mM EDTA

0.05% b-octylgucoside.

MR (C808S, S810L) LBD was bound to progesterone and spironolactone in the same protein buffer, but at a concentration of ˜5 mgs/ml.

Crystals were grown at room temperature in hanging drops containing 3.0 μl of the above protein buffer solution, and 0.5 μl of well buffer (50 mM HEPES, pH 7.5-8.5 (preferred pH range is 8.0 to 8.5), and 1.7-2.3M ammonium formate). Crystals were also obtained with mixing of the above protein solution and the well buffer at various volume ratios. Crystals appeared overnight and continuously grew to a size up to 300 μm within a week. Before data collection, crystals were transiently mixed with the well buffer that contained an additional 25% glycerol, and were then flash frozen in liquid nitrogen.

Crystallization of MR (C808S) LBD with deoxycorticosterone gave plate clusters in two conditions from the Index Screen (Hampton Research Corporation, Aliso Viejo, California). There were rods in 27 of the 96 wells, but the rods did not diffract.

Condition 1: 0.9M lithium sulphate, 0.1M HEPES pH 7.5, 2% polyetheleneglycol (PEG) 2000 monomethyl ether(2KMME).

Condition 2: 1.0M ammonium sulphate, 0.1M HEPES pH 7.0, 2% PEG 2KMME.

Condition 1(Li2SO4) was optimized and frozen by slow exchange. The slow exchange method consisted of placing the crystals in a three well depression slide in 50 ul of the following four solutions. The solution was pipetted away after ten minutes with solution 4 being used twice.

| 1 | 3 |
|---|---|
| 1.0M Lithium Sulphate | 50 μl of solution 1 |
| 0.1M Hepes pH 7.5 | 100 μl of solution 4 |
| 2% PEG 2KMME | |
| 2 | 4 |
| 100 μl of solution 1 | 1.0M Lithium Sulphate |
| 50 μl of solution 4 | 0.1M Hepes pH 7.5 |
| | 2% PEG 2KMME |
| | 25% Ethyleneglycol |

Diffracting crystals of deoxycorticosterone with TIF2 and aldosterone were obtained in the same conditions and frozen utilizing the same method. Diffracting crystals of MR (both single and double mutant) bound to progesterone and spironolactone (double mutant only) were obtained in the same conditions as MR-deoxycorticosterone crystals, micro-seeded with crushed MR-deoxycorticosterone crystals that were streaked with a horse tail hair.

### Example 9

### WT MR/deoxvcorticosterone Crvstal Structure

High amino acid sequence identity (57%) exists between the GR and MR LBDs. Like GR, bacterial expression of MR has historically proven difficult. However, using high levels (50 μM) of the potent ligand deoxycorticosterone in large volumes (24 L) of growth media, expression of the receptor was completed and wild type MR LBD was purified as described in the Examples herein above. The protein crystallized readily as described in the Examples herein above and the structure was determined as described herein. The WT MR/deoxycorticosterone crystal structure data is shown in Table 4.

TABLE 4
Summary of Data and Refinement Statistics

| Crystal | Native MR/deoxy |
|---|---|
| Space Group | C222₁ |
| Unit Cell | a = 93, b = 173.6, c = 42.4 |
| | α = β = γ = 90° |
| Resolution Range | 20.-2.36 |
| Observations (Unique) | 186628 (15013) |
| Completeness | 88.3 (34) |
| I/σ | 19.0 (2.5) |
| Rmerge % | 9.6 (35) |
| Refinement Statistics | |
| Resolution Range | 20.-2.36 |
| % Rfree | 7 |
| Rcryst Rfree | 24.0 (27.3) |
| Protein atoms | 1789 |
| Ligand atoms | 33 |
| Water Molecules | 43 |
| Rmsd bonds/angles | 0.0093/2.01 |

Rmerge = Σ|I − <I>|/ΣI
Rcryst = Σ|F_{obs} − F_{calc}|/ΣF_{obs}

### Example 10

### Crystal Structures of MR C808S and S81 0L Mutants with Progesterone

MR also binds progesterone with high affinity and crystal structures of the both MR C808S and MR S810L bound to progesterone were solved as described herein in the Examples above. Once again only minor differences are seen in the two structures. These structures are summarized in Table 5 below.

Expression of the receptor was completed and the MR LBD was purified as described in the Examples herein above. The protein was crystallized as described in the Examples herein above.

TABLE 5

| | MR | | MR | | MR | | MR | |
|---|---|---|---|---|---|---|---|---|
| Crystal | C808S/deoxy | | C808S/deoxy/TIF2 | | C808S/aldosterone | | C808S/progesterone | |
| Space Group | C222₁ | | P2₁ | | C222₁ | | P2₁2₁2₁ | |
| Unit Cell | a = 93.0, | | a = 40.1, b = 80.6, | | a = 92.5, | | a = 42.2, b = 89.4, | |
| | b = 173.4, | | c = 116.9 | | b = 173.2, | | c = 172.2 | |
| | c = 42.1 | | α = 90°, | | c = 42.2 | | α = β = γ = 90° | |
| | α = β = γ = 90° | | β = 94.24° γ = 90° | | α = β = γ = 90° | | | |
| Resolution | 20.-2.3 | | 20.-2.4 | | 20.-1.95 | | 20.-2.2 | |
| Range | | | | | | | | |
| Observations | 319581 | (16083) | 327936 | (25669) | 326513 | (25372) | 942996 | (33798) |
| (Unique) | | | | | | | | |
| Completeness | 94.1 | (83) | 94 | (69) | 90.4 | (60) | 97.8 | (95.1) |
| I/σ | 22.9 | (2.5) | 16.5 | (2.5) | 24.8 | (2.5) | 25.0 | (3.5) |
| Rmerge % | 8.8 | (37) | 7.6 | (41) | 6.7 | (37) | 8.5 | (40) |
| Refinement | | | | | | | | |
| Statistics | | | | | | | | |
| Resolution | 20.-2.2 | | 20.-2.4 | | 20.-1.95 | | 20.-2.2 | |
| Range | | | | | | | | |
| % Rfree | 7 | | 7 | | 7 | | 7 | |
| Rcryst Rfree | 22.4 | (26.4) | 22.4 | (26.9) | 21.4 | (23.5) | 22.0 | (26.4) |

| Protein atoms | 2045 | 4083 | 2019 | 4010 |
|---|---|---|---|---|
| Ligand atoms | 24 | 50 | 26 | 48 |
| Water | 182 | 131 | 144 | 255 |
| Molecules | | | | |
| Rmsd | 0.006/1.07 | 0.007/1.16 | 0.01/1.28 | 0.01/1.25 |
| bonds/angles | | | | |

| | MR double mutant | | MR double mutant | | MR double mutant | |
|---|---|---|---|---|---|---|
| Crystal | progesterone | | spironolactone | | cortisone | |
| Space Group | P2₁2₁2₁ | | P2₁2₁2₁ | | P2₁2₁2₁ | |
| Unit Cell | a = 42.2, b = 89.7, | | a = 42.2, b = 89.9, | | a = 42.3, b = 89.7, | |
| | c = 171.8 | | c = 171.8 | | c = 172.3 | |
| | α = β = γ = 90° | | α = β = γ = 90° | | α = β = γ = 90° | |
| Resolution Range | 20.-1.95 | | 20.-1.85 | | 20.-2.1 | |
| Observations (Unique) | 942996 | (33798) | 579086 | (56823) | 389157 | (40406) |
| Completeness | 97.8 | (95.1) | 96.5 | (83.7) | 82.7 | (35.5) |
| I/σ | 25.0 | (3.5) | 23.0 | (2.5) | 12.0 | (2.5) |
| Rmerge % | 8.5 | (40) | 6.3 | (40) | 8.0 | (38) |
| Refinement Statistics | | | | | | |
| Resolution Range | 20.-1.95 | | 20.-1.85 | | 20.-2.1 | |
| % Rfree | 7 | | 7 | | 7 | |
| Rcryst Rfree | 19.7 | (22.4) | 19.7 | (21.8) | 21.8 | (24.7) |

| | | | |
|---|---|---|---|
| Protein atoms | 4088 | 4112 | 4097 |
| Ligand atoms | 48 | 60 | 54 |
| Water Molecules | 325 | 428 | 379 |
| Rmsd bonds/angles | 0.01/1.35 | 0.009/1.26 | 0.009/1.06 |

Rmerge = Σ|I − <I>|/ΣI
Rcryst = Σ|F_{obs} − F_{calc}|/ΣF_{obs}

### REFERENCES

The references listed below as well as all references cited in the specification are incorporated herein by reference to the extent that they supplement, explain, provide a background for or teach methodology, techniques and/or compositions employed herein.
Altschul et al., (1990) J. Mol. Biol. 215: 403-10
Apriletti et al., (1995) Protein Expression and Purification, 6: 368-370
Arth et al., (1958) J. Am. Chem. Soc. 80: 3161
Ausubel et al., (1989) Current Protocols in Molecular Biology
Bartlett et al., (1989) Special Pub., Royal Chem. Soc. 78: 182-96
Beato, (1989) Cell 56:335-344
Bledsoe et al., (2002) Cell 110(1):93-105
Blundell & Johnson, (1985) Method.Enzymol., 114A & 115B
Bohm, (1992) J. Comput. Aid. Mol. Des., 6: 61-78
Brooks et al., (1983) J. Comp. Chem., 8: 132
Brünger, (1992) X-PLOR, Version 3.1. A System for X-ray Crystallography and NMR, Yale University Press, New Haven, Conn.
Case et al., (1997), AMBER 5, University of California, San Francisco
Cohen & Duke, (1984) J. Immunol. 152: 38-42
Cohen et al., (1990) J. Med. Chem. 33: 883-94
Crameri et al., Nature Biotechnology 14, 315-319.
Creighton, (1983) Proteins: Structures and Molecular Principles, W.H. Freeman & Co., New York Crystallography, Academic Press, San Diego, Calif.
Danielsen et al., (1987) Molec. Endocrinol. 1: 816-822
Danielsen et al., (1989) Cancer Res. 49: 2286s-2291s
DeBosscher et al., (2000) Proc. Natl. Acad. Sci. U S A 97: 3919-3924
Drewes et al., (1996) Mol. Cell. Biol. 16:925-31
Ducruix & Geige, (1992) Crystallization of Nucleic Acids and Proteins: A Practical Approach, IRL Press, Oxford, England
Dyda, F., et al., (1994) Science 266(5193):1981-6
Dzau et al., (1981) Circulation 63:645-651
Eastman-Reks & Vedeckis, (1986) Cancer Res. 46: 2457-2462
Eisen et al., (1994). Proteins 19: 199-221
Evans, (1988) Science 240:889-895
Evans, (1989) in Recent Progress in Hormone Research (Clark, ed.) Vol. 45, pp. 1-27, Academic Press, San Diego, California
Fried & Sabo, (1954) J. Am Chem. Soc. 76: 1455
Gampe et al., (2000) Mol. Cell 5: 545-55
Garabed ian & Yamamoto, (1992) Mol. Biol. Cell 3(11): 1245-57
Geller et al., (2000) Science 289(5476): 119-23
Gerbe et al., (1993) In Goodman and Gilman's the Pharmacological Basis of Therapeutics (Goodman & Gilman, eds.) 8t ed. p.784-813, McGraw-Hill, New York, N.Y.
Giguere et al., (1986) Cell 46: 645-652
Godowski et al., (1987) Nature 325: 365-368
Goodford, (1985) J. Med. Chem. 28: 849-57
Goodsell & Olsen, (1990) Proteins 8: 195-202
Green & Chambon, (1987) Nature 325: 75-78
Gribskov et al., (1986) Nucl. Acids. Res. 14: 6745
Gruol et al., (1989) Molec. Endocrinol. 3: 2119-2127
Harlow & Lane (1988) Antibodies: A Laboratory Manual, Cold Spring Harbor Laboratory Press, Cold Spring Harbor, N.Y.
Harmon et al., (1979) J. Cell Physiol. 98: 267-278
Hauptman, (1997) Cuff. Opin. Struct. Biol. 7: 672-80
Henikoff & Henikoff, (1989) Proc Natl Acad Sci U.S.A. 89: 10915
Hirschman et al. (1956) J. Am. Chem. Soc. 78: 4957
Hollenberg & Evans, (1988) Cell 55: 899-906
Hollenberg et al., (1987) Cell 49: 39-46
Hollenberg et al., (1989) Cancer Res. 49: 2292s-2294s
Homo-Delarche, (1984) Cancer Res. 44: 431-437
Janknecht, (1991) Proc. Natl. Acad. Sci. U.S.A. 88: 8972-8976
Jenkins et al., (2001) Trends Endocrinol. Metab. 12: 122-126
Jung et al., (1999) J. Mol. Biol. 294, 163-180
Karlin & Altschul, (1993) Proc NatlAcad Sci U.S.A. 90: 5873-5887
Kauppi et al., (2003) J. Biol. Chem. 278(25):22748-54
Kelso & Munck, (1984) J. Immunol. 133:784-791
Kuntz et al., (1992) J. Mol. Biol. 161: 269-88
Kyle & Doolittle, (1982), J. Mol. Biol. 157: 105-132
Lambert, (1997) in Practical Application of Computer-Aided Drug Design, (Charifson, ed.) Marcel-Dekker, New York, pp. 243-303
Lattman, (1985) Method Enzymol., 115: 55-77
Martin, (1992) J. Med. Chem. 35: 2145-54
McConkey et al., (1989) Arch. Biochem. Biophys. 269: 365-370
McKenna & O'Malley (2002) Endocrinology 143(7): 2461-2465
McPherson, (1982) Preparation and Analysis of Protein Crystals, John Wiley, New York
McPherson, (1990) Eur. J. Biochem. 189:1-23
McRee, (1992) J. Mol. Graphics 10: 44-46
McRee, (1993) Practical Protein Crystallography, Academic Press, New York
Miesfeld et al., (1987) Science 236:423-427
Miranker & Karplus, (1991) Proteins 11: 29-34
Navia & Murcko, (1992) Curr. Opin. Struc. Biol. 2: 202-10
Needleman et al., (1970) J. Mol. Biol. 48: 443
Nicholls et al., (1991) Proteins 11: 281
Nishibata & ltai, (1991) Tetrahedron 47: 8985
Nolte et al., (1998) Nature 395:137-43
Oberfield et al., Proc Natl Acad Sci U S A. (1999) May 25; 96(11):6102-6
Oliveto et al., (1958) J. Am. Chem. Soc. 4431
Oroetal., (1988) Cell 55: 1109-1114
Otwinowski & Minor (1997), Methods in Enzymology, Volume 276: Macromolecular Crystallography, part A, p. 307-326, 1997, C. W. Carter, Jr. & R. M. Sweet, Eds., Academic Press (New York).
Parks et al., (1999) Science 284: 1365-1368
Pearlman et al., (1995) Comput. Phys. Commun. 91: 1-41
Picard & Yamamoto, (1987) EMBO J. 6: 3333-3340
Picard etal., (1990) Cell Regul. 1: 291-299
Pjura & Matthews, (1993) Protein Science 2, 2226-2236
Quinkler & Stewart, (2003) J. Clin. Endocrinol. Metab. 88(6): 2384-92
Rafestin-Oblin et al., (2003) Endocrinology 144(2): 528-33
Rarey et al., (1996) J. Comput. Aid. Mol. Des. 10:41-54
Rocha et al., (1999) Am J Hypertension 12:76A
Rossmann, ed, (1972) The Molecular Replacement Method, Gordon & Breach, New York
Sack et al., 2001 Proc. Natl. Acad. Sci. U S A 98(9):4904-9
Sambrook et al., (1989) Molecular Cloning: A Laboratory Manual, Cold Spring Harbor Laboratory, New York
Schwartz et al., eds., (1979), Atlas of Protein Sequence and Structure. National Biomedical Research Foundation, pp. 357-358
Seielstad et al., (1995) Mol. Endocrinol. 9: 647-658
Sheldrick (1990) Acta Cryst. A46: 467
Shiau et al., (1998) Cell 95: 927-37
Sladek et al., Genes Dev. 4:2353-65
Smith et al., (1981) Adv. Appl. Math. 2:482
Struthers, (1996) J Cardiac Failure 2:47-54
Subramaniam et al., (1999) J. Biol. Chem. 274(25): 18121-7
Thompson, (1989) Cancer Res. 49: 2259s-2265s
Umesono & Evans, (1989) Cell 57: 1139-1146
Van Holde, (1971) Physical Biochemistry, Prentice-Hall, New Jersey, pp. 221-39
Voegel et al., (1998) EMBO J. 17: 507-519
Weber, (1991) Adv. Protein Chem. 41:1-36
Weber et al. (Cir 1987;75 (Supp. I)I-40 through I-47
Weeks et al., (1993) Acta Cryst. D49: 179
Wellner, (1971) Anal. Chem. 43: 5971
Wetmur & Davidson, (1968) J. Mol. Biol. 31: 349-70
Williams & Sigler, (1998) Nature 393(6683): 392-6
Wyckoff et al., eds., Academic Press
Yamamoto, (1985) Ann. Rev. Genet. 19: 209-252
Yuh & Thompson, (1989) J. Biol. Chem. 264: 10904-10910
Zhang et al., Nature (1997) May 8; 387(6629):206-9
Zhou, et al., (1998) Mol. Endocrinol. 12: 1594-1604
U.S. Pat. No. 3,007,923
U.S. Pat. No. 6,008,033
U.S. Pat. No. 4,196,265
U.S. Pat. No. 4,554,101
U.S. Pat. No. 5,260,203
U.S. Pat. No. 5,463,564
U.S. Pat. No. 5,834,228
U.S. Pat. No. 5,872,011
U.S. Pat. No. 6,236,946
PCT Patent App. No. WO 99/26966
PCT Patent App. No. WO 02/10143
PCT Patent App. No. WO 84/03564

It will be understood that various details of the presently disclosed subject matter can be changed without departing from the scope of the presently disclosed subject matter. Furthermore, the foregoing description is for the purpose of illustration only, and not for the purpose of limitation.

LENGTHY TABLE REFERENCED HERE
US20070219348A1-20070920-T00001
Please refer to the end of the specification for access instructions.

LENGTHY TABLE REFERENCED HERE
US20070219348A1-20070920-T00002
Please refer to the end of the specification for access instructions.

LENGTHY TABLE REFERENCED HERE
US20070219348A1-20070920-T00003
Please refer to the end of the specification for access instructions.

LENGTHY TABLE REFERENCED HERE
US20070219348A1-20070920-T00004
Please refer to the end of the specification for access instructions.

LENGTHY TABLE REFERENCED HERE
US20070219348A1-20070920-T00005
Please refer to the end of the specification for access instructions.

LENGTHY TABLE REFERENCED HERE
US20070219348A1-20070920-T00006
Please refer to the end of the specification for access instructions.

LENGTHY TABLE REFERENCED HERE
US20070219348A1-20070920-T00007
Please refer to the end of the specification for access instructions.

LENGTHY TABLE
The patent application contains a lengthy table section. A copy of the table is available in electronic form from the USPTO web site (http://seqdata.uspto.gov/?pageRequest=docDetail&DocID=US20070219348A1). An electronic copy of the table will also be available from the USPTO upon request and payment of the fee set forth in 37 CFR 1.19(b)(3).

What is claimed is:

1. A method of modeling one or more molecular interactions of a native NR with a ligand having low affinity for the native NR, the method comprising:
(a) crystallizing a surrogate ligand binding domain polypeptide in complex with a ligand having low affinity for a native NR to form a crystallized surrogate ligand binding domain polypeptide-ligand complex, wherein the surrogate ligand binding domain polypeptide comprises at least one mutation, and wherein the mutation improves ligand binding, crystal forming properties, or both ligand binding and crystal forming properties; and
(b) analyzing the crystallized complex to determine a three-dimensional structure of the crystallized complex, whereby the three-dimensional structure of the crystallized complex models one or more molecular interactions of the native NR with the ligand.

2. The method of claim 1, wherein the crystallizing is accomplished by the hanging drop method.

3. The method of claim 2, wherein the surrogate LBD polypeptide in complex with the ligand is mixed within a reservoir.

4. The method of claim 1, wherein the surrogate LBD polypeptide is an MR ligand binding domain polypeptide.

5. The method of claim 4, wherein the mutation is selected from the group consisting of C808S, S810L and combinations thereof.

6. The method of claim 5, wherein the mutation is both C808L and S810L.

7. The method of claim 4, wherein the MR ligand binding domain polypeptide has the amino acid sequence shown in any one of SEQ ID NOs:6, 8 and 10.

8. The method of claim 1, wherein the ligand is a non-steroidal ligand.

9. The method of claim 1, wherein the ligand is a steroid.

10. The method of claim 1, wherein the ligand is selected from the group consisting of aldosterone, deoxycorticosterone, progesterone, spironolactone and cortisone.

11. The method of claim 1, wherein the ligand has an IC50 binding affinity for the native NR of >50 nM.

12. The method of claim 1, wherein the native NR is an SR.

13. The method of claim 12, wherein the SR is a receptor selected from the group consisting of AR, PR, MR and GR.

14. The method of claim 1, wherein the crystallized complex comprises the surrogate ligand binding domain polypeptide, the ligand, and one of a co-activator or a co-repressor polypeptide.

15. The method of claim 14, wherein the ligand is a steroid and the polypeptide is a co-activator polypeptide.

16. The method of claim 15, wherein the steroid is aldosterone or deoxycorticosterone.

17. The method of claim 15, wherein the co-activator polypeptide is a TIF2 polypeptide fragment.

18. The method of claim 17, wherein the TIF2 polypeptide fragment has the amino acid sequence shown in SEQ ID NO:11.

19. The method of claim 14, wherein the ligand is a steroid and the polypeptide is a co-repressor polypeptide.


SEQUENCE LISTING (18 sequences)

SEQ ID NO: 1 - DNA, 5749 bp, Homo sapiens
cgcgggagccaacttcaggctgctcagaggaagcccgtgcagtcagtcacctgggtgcaagagcgttgctgcctcgggctctcccgctgcagggagagcggcactcgctggcctggatgtggttggatttaggggggctccgcagcaggggtttcgtggcggtggcaagcgctgcaacaggtagacggcgagagacggaccccggccgaggcagggatggagaccaaaggctaccacagtctccctgaaggtctagatatggaaagacggtggggtcaagtttctcaggctgtggagcgttcttccctgggacctacagagaggaccgatgagaataactacatggagattgtcaacgtaagctgtgtttccggtgctattccaaacaacagtactcaaggaagcagcaaagaaaaacaagaactactcccttgccttcagcaagacaataatcggcctgggattttaacatctgatattaaaactgagctggaatctaaggaactttcagcaactgtagctgagtccatgggtttatatatggattctgtaagagatgctgactattcctatgagcagcagaaccaacaaggaagcatgagtccagctaagatttatcagaatgttgaacagctggtgaaattttacaaaggaaatggccatcgtccttccactctaagttgtgtgaacacgcccttgagatcatttatgtctgactctgggagctccgtgaatggtggcgtcatgcgcgccattgttaaaagccctatcatgtgtcatgagaaaagcccgtctgtttgcagccctctgaacatgacatcttcggtttgcagccctgctggaatcaactctgtgtcctccaccacagccagctttggcagttttccagtgcacagcccaatcacccagggaactcctctgacatgctcccctaatgctgaaaatcgaggctccaggtcgcacagccctgcacatgctagcaatgtgggctctcctctctcaagtccgttaagtagcatgaaatcctcaatttccagccctccaagtcactgcagtgtaaaatctccagtctccagtcccaataatgtcactctgagatcctctgtgtctagccctgcaaatattaacaactcaaggtgctctgtttccagcccttcgaacactaataacagatccacgctttccagtccggcagccagtactgtgggatctatctgtagccctgtaaacaatgccttcagctacactgcttctggcacctctgctggatccagtacattgcgggatgtggttcccagtccagacacgcaggagaaaggtgctcaagaggtcccttttcctaagactgaggaagtagagagtgccatctcaaatggtgtgactggccagcttaatattgtccagtacataaaaccagaaccagatggagcttttagcagctcatgtctaggaggaaatagcaaaataaattcggattcttcattctcagtaccaataaagcaagaatcaaccaagcattcatgttcaggcacctcttttaaagggaatccaacagtaaacccgtttccatttatggatggctcgtatttttcctttatggatgataaagactattattccctatcaggaattttaggaccacctgtgcccggctttgatggtaactgtgaaggcagcggattcccagtgggtattaaacaagaaccagatgacgggagctattacccagaggccagcatcccttcctctgctattgttggggtgaattcaggtggacagtccttccactacaggattggtgctcaaggtacaatatctttatcacgatcggctagagaccaatctttccaacacctgagttcctttcctcctgtcaatactttagtggagtcatggaaatcacacggcgacctgtcgtctagaagaagtgatgggtatccggtcttagaatacattccagaaaatgtatcaagctctactttacgaagtgtttctactggatcttcaagaccttcaaaaatatgtttggtgtgtggggatgaggcttcaggatgccattatggggtagtcacctgtggcagctgcaaagttttcttcaaaagagcagtggaagggcaacacaactatttatgtgctggaagaaatgattgcatcattgataagattcgacgaaagaattgtcctgcttgcagacttcagaaatgtcttcaagctggaatgaatttaggagcacgaaagtcaaagaagttgggaaagttaaaagggattcacgaggagcagccacagcagcagcagcccccacccccacccccacccccgcaaagcccagaggaagggacaacgtacatcgctcctgcaaaagaaccctcggtcaacacagcactggttcctcagctctccacaatctcacgagcgctcacaccttcccccgttatggtccttgaaaacattgaacctgaaattgtatatgcaggctatgacagctcaaaaccagatacagccgaaaatctgctctccacgctcaaccgcttagcaggcaaacagatgatccaagtcgtgaagtgggcaaaggtacttccaggatttaaaaacttgcctcttgaggaccaaattaccctaatccagtattcttggatgtgtctatcatcatttgccttgagctggagatcgtacaaacatacgaacagccaatttctctattttgcaccagacctagtctttaatgaagagaagatgcatcagtctgccatgtatgaactatgccaggggatgcaccaaatcagccttcagttcgttcgactgcagctcacctttgaagaatacaccatcatgaaagttttgctgctactaagcacaattccaaaggatggcctcaaaagccaggctgcatttgaagaaatgaggacaaattacatcaaagaactgaggaagatggtaactaagtgtcccaacaattctgggcagagctggcagaggttctaccaactgaccaagctgctggactccatgcatgacctggtgagcgacctgctggaattctgcttctacaccttccgagagtcccatgcgctgaaggtagagttccccgcaatgctggtggagatcatcagcgaccagctgcccaaggtggagtcggggaacgccaagccgctctacttccaccggaagtgactgcccgctgcccagaagaactttgccttaagtttccctgtgttgttccacacccagaaggacccaagaaaacctgtttttaacatgtgatggttgattcacacttgttcaacagtttctcaagtttaaagtcatgtcagaggtttggagccgggaaagctgtttttccgtggatttggcgagaccagagcagtctgaaggattccccacctccaatcccccagcgcttagaaacatgttcctgttcctcgggatgaaaagccatatctagtcaataactctgattttgatattttcacagatggaagaagttttaactatgccgtgtagtttctggtatcgttcgcttgttttaaaagggttcaaggactaacgaacgttttaaagcttacccttggtttgcacataaaacgtatagtcaatatggggcattaatattcttttgttattaaaaaaacacaaaaaaataataaaaaaatatatacagattcctgttgtgtaataacagaactcgtggcgtggggcagcagctgcctctgagccctcgctcgtccacggtcttctgcatcactggtatacacactcgttagcgtccatttcttatttaattagaatggataagatgatgttaaatgccttggtttgatttctagtatctattgtgttggctttacaaataattttttgcagtcttttgctgtgctgtacattactgtatgtataaattatgaaggacctgaaataaggtataaggatcttttgtaaatgagacacatacaaaaaaaatctttaatggttaataggatgaatgggaaagtatttttgaaagaattctattttgctggagactatttaagtactatctttgtctaaacaaggtaatttttttttgtaaagtgcaatgtcctgcatgcataatgaaccgtttacagtgtatttaagaaagggaaagctgtgccttttttagcttcatatctaatttaccattattttacagtctctgttgtaaataaccacactgaaacctcttcggttgtcttgaaacctttctactttttctgtactttttgttttgttcttggtctcccgcttggggcatttgtgggactccagcacgttttctggcttctgcttcatcctgctccatcggggaatgacacactgcggtgtctgcagctcctggaaggtgtcatttgacaacacatgtgggagaggaggtccttggagtgctgcagctttgggaaagcctgcctcgtttcccttttcctctagaagcagaaccagctctacgagagtgagactgggaacttgatggctcagagagcatcttttcctcccattttagaaaatcagattttctcctgtgggaaaaaaaaattccatgcactctctctctgttaaagatcagctattcccttctgatcttggaaagaggttctgcactcctggaaccggtcacaggaacgcacagatcatggcaggatgcgctgggacggcccatcttggcaaggttcagtctgaatggcatggagaccgggagatagaggggttttagatttttaaaaggtaggttttaaaaataagttttatacataaacagttttggagaaaaattacagatcatataagcaagacagtggcactaaaatgtttaattcattaatctgtttgtttggcactgatgcaatgtatggcttttctcttgccccaaatcacaaacatatgtatctttggggaaactaacaatatgattgcactaaataaactactttgaatagaggccaaattaatcttttaaaaatgatgataatcatcaggtttactcagtgaaatcatattaattattttccaaaatctaaaagctgtagctggagaagcccatggccacgaggaagcagcaattaattagatcaacacttttctccagggttcaccatgcaggcaacattaccttgtctttcaaaagacacctgccttagtgcaaggggaaacctgtgaaagctgcactcagagggaggagtctttcttacataatttgcaatttcaggaatttaatttataggcagatctttaaatacagtcaacttacggtgcacagtaatatgaaagccacactttgaaggtaataaatacacagcatgcagactgggagttgctagcaaacaaatggcttacttacaaaagcagcttttagttcagacttagtttttataaaatgagaattctgacttacttaaccaggtttgggatggagatggtctgcatcagctttttgtattaacaaagttactggctctttgtgtgtctccaggtaactttgcttgattaaacagcaaagccatattctaaattcactgttgaatgcctgtcccagtccaaattgtctgtctgctcttatttttgtaccatattgctcttaaaaatcttggtttggtacagttcataattcaccaaaaagttcatataatttaaagaaacactaaattagtttaaaatgaagcaatttatatctttatgcaaaaacatatgtctgtctttgcaaaggactgtaagcagattacaataaatcctttacttt

SEQ ID NO: 2 - protein, 984 aa, Homo sapiens
METKGYHSLPEGLDMERRWGQVSQAVERSSLGPTERTDENNYMEIVNVSCVSGAIPNNSTQGSSKEKQELLPCLQQDNNRPGILTSDIKTELESKELSATVAESMGLYMDSVRDADYSYEQQNQQGSMSPAKIYQNVEQLVKFYKGNGHRPSTLSCVNTPLRSFMSDSGSSVNGGVMRAIVKSPIMCHEKSPSVCSPLNMTSSVCSPAGINSVSSTTASFGSFPVHSPITQGTPLTCSPNAENRGSRSHSPAHASNVGSPLSSPLSSMKSSISSPPSHCSVKSPVSSPNNVTLRSSVSSPANINNSRCSVSSPSNTNNRSTLSSPAASTVGSICSPVNNAFSYTASGTSAGSSTLRDVVPSPDTQEKGAQEVPFPKTEEVESAISNGVTGQLNIVQYIKPEPDGAFSSSCLGGNSKINSDSSFSVPIKQESTKHSCSGTSFKGNPTVNPFPFMDGSYFSFMDDKDYYSLSGILGPPVPGFDGNCEGSGFPVGIKQEPDDGSYYPEASIPSSAIVGVNSGGQSFHYRIGAQGTISLSRSARDQSFQHLSSFPPVNTLVESWKSHGDLSSRRSDGYPVLEYIPENVSSSTLRSVSTGSSRPSKICLVCGDEASGCHYGVVTCGSCKVFFKRAVEGQHNYLCAGRNDCIIDKIRRKNCPACRLQKCLQAGMNLGARKSKKLGKLKGIHEEQPQQQQPPPPPPPPQSPEEGTTYIAPAKEPSVNTALVPQLSTISRALTPSPVMVLENIEPEIVYAGYDSSKPDTAENLLSTLNRLAGKQMIQVVKWAKVLPGFKNLPLEDQITLIQYSWMCLSSFALSWRSYKHTNSQFLYFAPDLVFNEEKMHQSAMYELCQGMHQISLQFVRLQLTFEEYTIMKVLLLLSTIPKDGLKSQAAFEEMRTNYIKELRKMVTKCPNNSGQSWQRFYQLTKLLDSMHDLVSDLLEFCFYTFRESHALKVEFPAMLVEIISDQLPKVESGNAKPLYFHRK

SEQ ID NO: 3 - DNA, 819 bp, Homo sapiens
gctcctgcaaaagaaccctcggtcaacacagcactggttcctcagctctccacaatctcacgagcgctcacaccttcccccgttatggtccttgaaaacattgaacctgaaattgtatatgcaggctatgacagctcaaaaccagatacagccgaaaatctgctctccacgctcaaccgcttagcaggcaaacagatgatccaagtcgtgaagtgggcaaaggtacttccaggatttaaaaacttgcctcttgaggaccaaattaccctaatccagtattcttggatgtgtctatcatcatttgccttgagctggagatcgtacaaacatacgaacagccaatttctctattttgcaccagacctagtctttaatgaagagaagatgcatcagtctgccatgtatgaactatgccaggggatgcaccaaatcagccttcagttcgttcgactgcagctcacctttgaagaatacaccatcatgaaagttttgctgctactaagcacaattccaaaggatggcctcaaaagccaggctgcatttgaagaaatgaggacaaattacatcaaagaactgaggaagatggtaactaagtgtcccaacaattctgggcagagctggcagaggttctaccaactgaccaagctgctggactccatgcatgacctggtgagcgacctgctggaattctgcttctacaccttccgagagtcccatgcgctgaaggtagagttccccgcaatgctggtggagatcatcagcgaccagctgcccaaggtggagtcggggaacgccaagccgctctacttccaccggaag

SEQ ID NO: 4 - protein, 273 aa, Homo sapiens
APAKEPSVNTALVPQLSTISRALTPSPVMVLENIEPEIVYAGYDSSKPDTAENLLSTLNRLAGKQMIQVVKWAKVLPGFKNLPLEDQITLIQYSWMCLSSFALSWRSYKHTNSQFLYFAPDLVFNEEKMHQSAMYELCQGMHQISLQFVRLQLTFEEYTIMKVLLLLSTIPKDGLKSQAAFEEMRTNYIKELRKMVTKCPNNSGQSWQRFYQLTKLLDSMHDLVSDLLEFCFYTFRESHALKVEFPAMLVEIISDQLPKVESGNAKPLYFHRK

SEQ ID NO: 5 - DNA, 819 bp, Homo sapiens
gctcctgcaaaagaaccctcggtcaacacagcactggttcctcagctctccacaatctcacgagcgctcacaccttcccccgttatggtccttgaaaacattgaacctgaaattgtatatgcaggctatgacagctcaaaaccagatacagccgaaaatctgctctccacgctcaaccgcttagcaggcaaacagatgatccaagtcgtgaagtgggcaaaggtacttccaggatttaaaaacttgcctcttgaggaccaaattaccctaatccagtattcttggatgagtctatcatcatttgccttgagctggagatcgtacaaacatacgaacagccaatttctctattttgcaccagacctagtctttaatgaagagaagatgcatcagtctgccatgtatgaactatgccaggggatgcaccaaatcagccttcagttcgttcgactgcagctcacctttgaagaatacaccatcatgaaagttttgctgctactaagcacaattccaaaggatggcctcaaaagccaggctgcatttgaagaaatgaggacaaattacatcaaagaactgaggaagatggtaactaagtgtcccaacaattctgggcagagctggcagaggttctaccaactgaccaagctgctggactccatgcatgacctggtgagcgacctgctggaattctgcttctacaccttccgagagtcccatgcgctgaaggtagagttccccgcaatgctggtggagatcatcagcgaccagctgcccaaggtggagtcggggaacgccaagccgctctacttccaccggaag

SEQ ID NO: 6 - protein, 273 aa, Homo sapiens
APAKEPSVNTALVPQLSTISRALTPSPVMVLENIEPEIVYAGYDSSKPDTAENLLSTLNRLAGKQMIQVVKWAKVLPGFKNLPLEDQITLIQYSWMSLSSFALSWRSYKHTNSQFLYFAPDLVFNEEKMHQSAMYELCQGMHQISLQFVRLQLTFEEYTIMKVLLLLSTIPKDGLKSQAAFEEMRTNYIKELRKMVTKCPNNSGQSWQRFYQLTKLLDSMHDLVSDLLEFCFYTFRESHALKVEFPAMLVEIISDQLPKVESGNAKPLYFHRK

SEQ ID NO: 7 - DNA, 819 bp, Homo sapiens
gctcctgcaaaagaaccctcggtcaacacagcactggttcctcagctctccacaatctcacgagcgctcacaccttcccccgttatggtccttgaaaacattgaacctgaaattgtatatgcaggctatgacagctcaaaaccagatacagccgaaaatctgctctccacgctcaaccgcttagcaggcaaacagatgatccaagtcgtgaagtgggcaaaggtacttccaggatttaaaaacttgcctcttgaggaccaaattaccctaatccagtattcttggatgtgtctattatcatttgccttgagctggagatcgtacaaacatacgaacagccaatttctctattttgcaccagacctagtctttaatgaagagaagatgcatcagtctgccatgtatgaactatgccaggggatgcaccaaatcagccttcagttcgttcgactgcagctcacctttgaagaatacaccatcatgaaagttttgctgctactaagcacaattccaaaggatggcctcaaaagccaggctgcatttgaagaaatgaggacaaattacatcaaagaactgaggaagatggtaactaagtgtcccaacaattctgggcagagctggcagaggttctaccaactgaccaagctgctggactccatgcatgacctggtgagcgacctgctggaattctgcttctacaccttccgagagtcccatgcgctgaaggtagagttccccgcaatgctggtggagatcatcagcgaccagctgcccaaggtggagtcggggaacgccaagccgctctacttccaccggaag

SEQ ID NO: 8 - protein, 273 aa, Homo sapiens
APAKEPSVNTALVPQLSTISRALTPSPVMVLENIEPEIVYAGYDSSKPDTAENLLSTLNRLAGKQMIQVVKWAKVLPGFKNLPLEDQITLIQYSWMCLLSFALSWRSYKHTNSQFLYFAPDLVFNEEKMHQSAMYELCQGMHQISLQFVRLQLTFEEYTIMKVLLLLSTIPKDGLKSQAAFEEMRTNYIKELRKMVTKCPNNSGQSWQRFYQLTKLLDSMHDLVSDLLEFCFYTFRESHALKVEFPAMLVEIISDQLPKVESGNAKPLYFHRK

SEQ ID NO: 9 - DNA, 819 bp, Homo sapiens
gctcctgcaaaagaaccctcggtcaacacagcactggttcctcagctctccacaatctcacgagcgctcacaccttcccccgttatggtccttgaaaacattgaacctgaaattgtatatgcaggctatgacagctcaaaaccagatacagccgaaaatctgctctccacgctcaaccgcttagcaggcaaacagatgatccaagtcgtgaagtgggcaaaggtacttccaggatttaaaaacttgcctcttgaggaccaaattaccctaatccagtattcttggatgtctctattatcatttgccttgagctggagatcgtacaaacatacgaacagccaatttctctattttgcaccagacctagtctttaatgaagagaagatgcatcagtctgccatgtatgaactatgccaggggatgcaccaaatcagccttcagttcgttcgactgcagctcacctttgaagaatacaccatcatgaaagttttgctgctactaagcacaattccaaaggatggcctcaaaagccaggctgcatttgaagaaatgaggacaaattacatcaaagaactgaggaagatggtaactaagtgtcccaacaattctgggcagagctggcagaggttctaccaactgaccaagctgctggactccatgcatgacctggtgagcgacctgctggaattctgcttctacaccttccgagagtcccatgcgctgaaggtagagttccccgcaatgctggtggagatcatcagcgaccagctgcccaaggtggagtcggggaacgccaagccgctctacttccaccggaag

SEQ ID NO: 10 - protein, 273 aa, Homo sapiens
APAKEPSVNTALVPQLSTISRALTPSPVMVLENIEPEIVYAGYDSSKPDTAENLLSTLNRLAGKQMIQVVKWAKVLPGFKNLPLEDQITLIQYSWMSLLSFALSWRSYKHTNSQFLYFAPDLVFNEEKMHQSAMYELCQGMHQISLQFVRLQLTFEEYTIMKVLLLLSTIPKDGLKSQAAFEEMRTNYIKELRKMVTKCPNNSGQSWQRFYQLTKLLDSMHDLVSDLLEFCFYTFRESHALKVEFPAMLVEIISDQLPKVESGNAKPLYFHRK

SEQ ID NO: 11 - protein, 25 aa, Homo sapiens
QEPVSPKKKENALLRYLLDKDDTKD

SEQ ID NO: 12 - protein, 5 aa, Homo sapiens
LRYLL

SEQ ID NO: 13 - DNA, 32 bp, Artificial
  5′ forward primer DNA oligonucleotide for MR LBD (C808S) mutant
gtattcttggatgtctctatcatcatttgcct

SEQ ID NO: 14 - DNA, 32 bp, Artificial
  3′ reverse primer DNA oligonucleotide for MR LBD (C808S) mutant
aggcaaatgatgatagagacatccaagaatac

SEQ ID NO: 15 - DNA, 28 bp, Artificial
  5′ forward primer DNA oligonucleotide for MR LBD (C808S, S810L) mutant
tcttggatgtctctattatcatttgcct

SEQ ID NO: 16 - DNA, 28 bp, Artificial
  3′ reverse primer DNA oligonucleotide for MR LBD (C808S, S810L) mutant
aggcaaatgataatagagacatccaaga

SEQ ID NO: 17 - protein, 275 aa, Homo sapiens
GSAPAKEPSVNTALVPQLSTISRALTPSPVMVLENIEPEIVYAGYDSSKPDTAENLLSTLNRLAGKQMIQVVKWAKVLPGFKNLPLEDQITLIQYSWMSLSSFALSWRSYKHTNSQFLYFAPDLVFNEEKMHQSAMYELCQGMHQISLQFVRLQLTFEEYTIMKVLLLLSTIPKDGLKSQAAFEEMRTNYIKELRKMVTKCPNNSGQSWQRFYQLTKLLDSMHDLVSDLLEFCFYTFRESHALKVEFPAMLVEIISDQLPKVESGNAKPLYFHRK

SEQ ID NO: 18 - protein, 275 aa, Homo sapiens
GSAPAKEPSVNTALVPQLSTISRALTPSPVMVLENIEPEIVYAGYDSSKPDTAENLLSTLNRLAGKQMIQVVKWAKVLPGFKNLPLEDQITLIQYSWMSLLSFALSWRSYKHTNSQFLYFAPDLVFNEEKMHQSAMYELCQGMHQISLQFVRLQLTFEEYTIMKVLLLLSTIPKDGLKSQAAFEEMRTNYIKELRKMVTKCPNNSGQSWQRFYQLTKLLDSMHDLVSDLLEFCFYTFRESHALKVEFPAMLVEIISDQLPKVESGNAKPLYFHRK